(19) United States
(12) Patent Application Publication
(10) Pub. No.: US 2019/0024132 A1
(43) Pub. Date: Jan. 24, 2019

(54) D-ALA-D-ALA-BASED DIPEPTIDES AS TOOLS FOR IMAGING PEPTIDOGLYCAN BIOSYNTHESIS

(71) Applicant: INDIANA UNIVERSITY RESEARCH AND TECHNOLOGY CORPORATION, Indianapolis, IN (US)

(72) Inventors: Michael S. Van Nieuwenhze, Bloomington, IN (US); Yves S. Brun, Bloomington, IN (US); Erkin Kuru, Bloomington, IN (US)

(21) Appl. No.: 16/048,024

(22) Filed: Jul. 27, 2018

Related U.S. Application Data
(63) Continuation of application No. 15/021,599, filed on Mar. 11, 2016.
(60) Provisional application No. 61/876,710, filed on Sep. 11, 2013.

(51) Int. Cl. C12Q 1/02; C07K 5/103; C07K 14/47; C12Q 1/10; G01N 33/58; C12Q 1/14; C12Q 1/04; C07K 5/083; G01N 33/569; G01N 33/50; C07K 5/062; C07K 7/06

(52) U.S. Cl. CPC C12Q 1/02; C07K 5/1008; C07K 7/06; C12Q 1/025; G01N 2333/32; C12Q 1/10; G01N 33/582; G01N 2333/245; G01N 2333/3156; C12Q 1/14; C12Q 1/04; C07K 5/0806; G01N 2500/10; G01N 2333/4722; G01N 33/56911; G01N 33/5035; C07K 5/06026; C07K 14/4725

(57) ABSTRACT

Disclosed herein are compositions for assessing peptidoglycan biosynthesis in bacteria using modified dipeptides containing a bioorthogonal tag and applying novel post-labeling methods to label the bioorthogonal tag. The resultant, labeled peptidoglycan structures are amenable for identifying bacteria by microscopic visualization.

### CROSS-REFERENCE TO RELATED APPLICATIONS

The present application is a continuation of U.S. patent application Ser. No. 15/021,599, filed Mar. 11, 2016, which is the National Stage of International Application No. PCT/US14/55177, filed Sep. 11, 2014, and entitled “D-ALA-D-ALA-BASED DIPEPTIDES AS TOOLS FOR IMAGING PEPTIDOGLYCAN BIOSYNTHESIS,” which claims the benefit of priority under 35 U.S.C. 119 to U.S. provisional patent application Ser. No. 61/876,710, filed Sep. 11, 2013, and entitled “D-ALA-D-ALA-BASED DIPEPTIDES AS TOOLS FOR IMAGING PEPTIDOGLYCAN BIOSYNTHESIS,” the contents of which are herein incorporated by reference in their entireties.

### STATEMENT REGARDING FEDERALLY SPONSORED RESEARCH OR DEVELOPMENT

This invention was made with government support under AI059327 and GM051986 awarded by the National Institutes of Health. The government has certain rights in the invention.

### 1. TECHNICAL FIELD

The present disclosure relates to modified dipeptides for incorporation into bacterial cell wall peptidoglycans and their use in post-labeling methods to visualize peptidoglycan biosynthesis by light microscopy.

### 2. DESCRIPTION OF RELATED ART

Bacterial growth is controlled by the domain-specific peptidoglycan (PG) cell wall, a rigid and essential structure composed of glycan strands cross-linked by D-amino acid (DAA)-containing short peptides, whose biosynthesis machinery is a target for antibiotics.

Despite the importance of PG, knowledge of its dynamics has been severely hampered by lack of a strategy for direct imaging of sites of PG biosynthesis in live cells. Significant limitations of current labeling methods, such as toxic effects and poor membrane permeability of the probes, have limited their applicability to only a small set of bacterial species. Moreover, these methods are labor-intensive and their sensitivity suffers from their indirect and multiple-step nature.

Methods relying on fluorescently labeled antibiotics to study bacterial cell wall synthesis and to discover new antibiotics to which bacteria remain susceptible have had a profound impact on the field. The current methods, however, have at least two inherent limitations. First, antibiotic concentration needs to be carefully controlled to avoid damage to the cell. Second, because these agents bind to specific sites on cell surfaces, they only will appear at sites of active PG biosynthesis.

The present inventors have disclosed previously a class of fluorescently-modified D-amino acids (FDAAs) that have enabled the visualization of peptidoglycan synthesis dynamics in live bacterial cells. See International Patent Application No. PCT/US13/37504, filed Apr. 21, 2013, to INDIANA UNIVERSITY RESEARCH & TECHNOLOGY CORPORATION, entitled COMPOSITIONS FOR IN SITU LABELING OF BACTERIAL CELL WALLS WITH FLUOROPHORES AND METHODS OF USE THEREOF.

Previous efforts directed toward development of imaging agents to visualize peptidoglycan biosynthesis have largely relied upon fluorescently modified cell wall acting antibiotics, or upon utilization of chemically modified cell wall precursors. Both methods have significant limitations due to toxicity and poor membrane permeability. To address these limitations, a series of modified D-Ala-D-Ala (DA-DA) dipeptides have been developed as novel substrates for peptidoglycan synthesis that permit post-labeling of the resultant peptidoglycans for visualization. These dipeptides, once incorporated into peptidoglycan and displayed on the cell surface, can readily capture fluorescent probes containing complementary functional groups, which are visualized by microscopy.

### BRIEF SUMMARY

In a first aspect, a modified dipeptide is disclosed that includes D-amino acids covalently attached to a bioorthogonal tag.

In a second aspect, a muramylpentapeptide precursor unit is disclosed that includes an N-acetyl muramic acid (NAM) moiety having a stem peptide of three to five amino acids. One or more of the dipeptides in the stem peptide includes a modified dipeptide that includes D-amino acids covalently attached to an orthogonal tag and optionally an additional modified dipeptide, wherein the additional modified dipeptide includes a clickable D-amino acid.

In a third aspect, a peptidoglycan unit is disclosed that includes a muramylpentapeptide precursor unit as described above in the second respect that is covalently linked to an N-acetyl glucosamine (NAG) moiety.

In a fourth aspect, a method of assessing bacterial cell wall synthesis in real time is described. The method includes the step of providing live bacteria with a first amount of at least one modified dipeptide comprising D-amino acids covalently attached to a bioorthogonal tag, and optionally a second amount of at least one additional modified dipeptide that includes a clickable D-amino acid, under conditions sufficient for bacterial cell wall synthesis, wherein the bacteria covalently incorporate the at least one modified amino acid and optionally the at least one additional modified dipeptide into a stem peptide of peptidoglycan of the bacterial cell wall.

In a fifth aspect, a method of screening for a putative cell wall-acting agent is disclosed. The method includes the step of co-contacting bacteria with an effective amount of an agent and an amount of at least one modified dipeptide comprising D-amino acids covalently attached to a bioorthogonal tag, and optionally an amount at least one additional modified dipeptide that includes a clickable D-amino acid, under conditions sufficient to permit ongoing peptidoglycan biosynthesis in a bacterial cell wall, wherein the agent comprises a cell wall-acting agent if the agent interferes with ongoing peptidoglycan biosynthesis in the bacterial cell wall.

In a sixth aspect, a method of screening for a putative cell wall-disrupting agent is disclosed. The method includes the step contacting modified bacteria with an amount of an agent. The agent is a cell wall-disrupting agent if the agent weakens integrity of peptidoglycan in an existing bacterial cell wall. In this method, the modified bacteria have a modified cell wall containing modified peptidoglycan having at least one stem peptide containing at least one modified dipeptide comprising D-amino acids covalently attached to a bioorthogonal tag, and optionally at least one additional modified dipeptide that includes a clickable D-amino acid.

In a seventh aspect, a method of identifying bacteria is disclosed. The method includes three steps. The first step includes contacting live bacteria with an amount of at least one modified dipeptide comprising D-amino acids covalently attached to a bioorthogonal tag, and optionally an amount of at least one additional modified dipeptide that includes a clickable D-amino acid, under conditions sufficient for ongoing bacterial cell wall synthesis. The bacteria covalently incorporate into peptidoglycan of a bacterial cell wall the at least one modified dipeptide, and optionally the at least one additional modified dipeptide. Each of the least one modified dipeptide and optionally the at least one additional modified dipeptide comprises a distinct bioorthogonal tag. The second step includes post-labeling each distinct bioorthogonal tag with a spectrally distinct label. The third step includes visualizing the spectrally distinct labels to determine an incorporation pattern of the at least one modified amino acid, and optionally the at least one additional modified amino acid, wherein the incorporation pattern identifies the bacteria.

In an eighth aspect, a kit for incorporating modified dipeptides into live bacteria is disclosed. The kit includes at least one modified dipeptide comprising D-amino acids covalently attached to a bioorthogonal tag and a positive bacterial control. The kit can include an optional negative bacterial control. The positive bacterial control has at least one modified dipeptide comprising D-amino acids covalently attached to a bioorthogonal tag incorporated into a stem peptide of peptidoglycan of the bacterial cell wall. The optional negative bacterial control, if included, does not have the modified dipeptide comprising D-amino acid covalently attached to a bioorthogonal tag incorporated into a stem peptide of peptidoglycan of the bacterial cell wall.

These and other features, objects and advantages of the present invention will become better understood from the description that follows. In the description, reference is made to the accompanying drawings, which form a part hereof and in which there is shown by way of illustration, not limitation, embodiments of the invention.

### BRIEF DESCRIPTION OF THE DRAWINGS

The features, objects and advantages other than those set forth above will become more readily apparent when consideration is given to the detailed description below. Such detailed description makes reference to the following drawings.

FIG. 1 shows the three general stages of PG biosynthesis and general structures of the NAM and NAG units of PG.

FIG. 2 shows exemplary D-Ala-based FDAAs. D-NBD and D-HCC (based on (R)-diaminopropionic acid) emit in the green and blue regions, respectively.

FIG. 3 shows results of control experiments in which the cell walls of Agrobacterium tumefaciens (“A. tumefaciens”; panels (i) and (ii)), Bacillus subtilis (“B. subtilis”; panel (iii)) and Escherichia coli (“E. coli”; panel (iv)) were fluorescently labeled with fluorescent D-Ala (D-HCC) or fluorescent L-Ala (L-HCC). An exemplary structure for D-Ala (D-HCC) also is shown in panel (v).

FIG. 4 shows results of a pulse chase experiment with a fluorescent D-Ala in B. subtilis ΔdacA (panel (i)) or A. tumefaciens (panel (ii)).

FIG. 5 shows results of a short pulse experiment with fluorescent D-Ala in B. subtilis.

FIG. 6 shows results of a fluorescent D-Ala derivative in a dual-labeling format.

FIG. 7 shows exemplary structures for FDAAs, such as HCC-OH-labeled 3-amino-D-Ala (HADA), NBD-Cl-labeled 3-amino-D-Ala (NADA), F-labeled D-Lys (FDL) and T-labeled D-Lys (TDL), as well as exemplary structures for CDAAs, such as EDA and ADA.

FIG. 8 shows that long labeling pulses with HADA uniformly label PG in live E. coli (“Escherichia coli”; left), B. subtilis (“Bacillus subtilis”; center) and A. tumefaciens (“Agrobacterium tumefaciens”; left). The FDAA fluorescence was retained in isolated sacculi, which also stained with a NAG-specific wheat germ agglutinin (WAG) lectin conjugated to Alexa Fluor® 594 (red). Scale bars, 2 μm.

FIG. 9A shows a schematic representing the muramylpentapeptide precursor as incorporated into a nascent PG unit and a modified D-amino acid (FDAA).

FIG. 9B shows HPLC detection of modified muropeptides in E. coli PG incubated with HADA, HALA and NADA, or NALA. Samples were monitored using a dual wavelength UV monitor set for general muropeptide detection and for FDAA-specific wavelengths. Peaks HEC-1 (panel (i)) and NEC-1 (panel (ii)) correspond to the HADA- or NADA-modified muropeptides in E. coli that were further characterized by electrospray ionization MS/MS (ESI-MS/MS).

FIG. 9C shows percentage of FDAA incorporation into the total muropeptides varies among bacterial PG, as revealed by HPLC analysis.

FIG. 9D shows a schematic representing MS/MS analyses of FDAAs exclusively incorporated into the 5th position in B. subtilis PG (panel (i)) and the 4th position of muropeptides in E. coli PG and A. tumefaciens PG (panel (ii)).

FIG. 10A shows time-lapse microscopy of HADA-labeled E. coli (panel (i)) and B. subtilis ΔdacA (panel (ii)) cells imaged during growth on LB agarose pads. White scale bars, 2 μm.

FIG. 10B shows super-resolution microscopy of E. coli after short pulses with HADA. Red scale bars, 1 μm.

FIG. 10C show super-resolution microscopy of A. tumefaciens after short pulses with HADA. Red scale bar, 1 μm.

FIG. 10D shows super-resolution microscopy of S. aureus after a short pulse with HADA. Autofluorescence is shown in red. Red scale bar, 1 μm.

FIG. 10E shows triple labeling of A. tumefaciens with HADA (blue), EDA (clicked with red sulfo-Cy3-azide) and NADA (green). Arrows in the triple labeling panel indicate the sequence of labeling. Red scale bar, 1 μm.

FIG. 10F shows triple labeling of S. venezuelae with NADA (green), TDL (red) and HADA (blue). Arrows in the triple labeling panel indicate the sequence of labeling. White scale bar, 2 μm.

FIG. 11 shows that short pulses of HADA label distinct modes of growth in diverse bacteria. Strains were labeled for ˜2%-8% of the doubling time: E. coli (30 seconds), A. tumefaciens (2 minutes), B. subtilis ΔdacA (30 seconds), S. aureus (2 minutes), L. lactis (2 minutes), S. pneumoniae (4 minutes), C. crescentus (5 minutes), Synechocystis sp. PCC 6803 (1 hour), S. venezuelae (2 minutes), B. conglomeratum (8 minutes), B. phytofirmans (20 minutes), V. Spinosum (10 minutes). Scale bars, 2 μm.

FIG. 12 shows a schematic for sequentially incorporating distinct FDAAs, such as NADA, TDL and HADA, into newly synthesized PG in live bacteria.

FIG. 13 depicts the chemical structures of D-alanine (D-Ala); D-alanyl-D-alanine dipeptide (DA-DA); ethynyl-D-alanine (EDA); ethynyl-D-alanyl-D-alanine (EDA-DA); D-alanyl-ethynyl-D-alanine dipeptide (DA-EDA); azido-D-alanine (ADA); azido-D-alanyl-D-alanine (ADA-DA); D-alanyl-azido-D-alanine (DA-ADA).

FIG. 14A depicts one embodiment of a dipeptide PG labeling strategy based upon biosynthesis of the terminal PG stem peptide of Gram-negative bacteria. Two D-alanines are first ligated together by D-alanine-D-alanine ligase and the dipeptide is subsequently added to the stem tripeptide by MurF, resulting in a pentapeptide. The labeling strategy relies on the inherent tolerance of the PG machinery to accept DA-DA analogues. Abbreviations: Alr, alanine racemase; DA-DA, D-alanyl-D-alanine; m-DAP, meso-diaminopimelic acid; Ddl, D-alanine-D-alanine ligase; EDA, ethynyl-D-alanine; EDA-DA, ethynyl-D-alanyl-D-alanine; D-Glu, D-Glutamic Acid; MurF, UDP-N-acetylmuramoyl-tripeptide-D-alanyl-D-alanine ligase; and MurNAc, N-acetylmuramic acid.

FIG. 14B depicts subsequent crosslinking between neighbouring peptide stems is carried out by a series of transpeptidases (penicillin-binding proteins). Upon transpeptidation, a proximal m-DAP from a neighbouring peptide stem attacks the carbonyl group between the penultimate and terminal D-alanines of the PG stem. The terminal D-alanine is thus cleaved from the stem peptide, which results in a tetrapeptide. Another pathway for the loss of terminal D-alanine is D,D-carboxypeptidation catalysed by enzymes such as DacA. Abbreviations: GlcNAc, N-acetylglucosamine; MurNAc, N-acetylmuramic acid; PBPs, penicillin-binding proteins; and DacA, D-alanyl-D-alanine carboxypeptidase A.

FIG. 15A depicts phase contrast and epifluorescence microscopy of E. coli cells labeled with 0.5 mM alkyne-containing EDA (panels (i) and (iv)), EDA-DA (panels (ii) and (v)), DA-EDA (panels (iii) and (vi)) for 5 min (panels (i)-(iii)) and 60 min (panels (iv)-(vi)). Panel (c) denotes control cell labeling. These samples together with unlabeled controls were ‘clicked’ to Alexa Fluor 488 azide and imaged. When the alkyne is on the C terminus (DA-EDA), the labeling is not apparent. Signal from N-terminally tagged dipeptide (EDA-DA) is significantly higher, but still lower than EDA and the patterns of labeling at the earlier time points are different. This is probably due to periplasmic incorporation of D-amino acids (for example, EDA) by E. coli L,D-transpeptidases, which result in more efficient peripheral labelling in addition to labelling due to lipid II-dependent PG synthesis. Therefore, in bacteria that have active L,D-transpeptidases, the cytoplasmic PG labeling through dipeptide probes provides a better measure of lipid II-dependent PG synthesis than single D-amino acids. The experiment was conducted twice and images are representative of a minimum of five fields viewed per condition/time point per replicate.

FIG. 15B depicts phase contrast and epifluorescence microscopy of E. coli cells labeled with 0.5 mM alkyne-containing EDA-DA (panel (i)) or the L-enantiomer control ethynyl-L-alanine-L-alanine (ELA-LA) (panel (ii)) for 45 min and clicked as described in FIG. 15A. The comparison of FIGS. 15A, B shows that the labeling is D-enantiomer-specific. Images are representative of a minimum of four fields viewed per replicate and the experiment was conducted twice.

FIG. 16A depicts an exemplary embodiment of phase contrast and epifluorescence microscopy of B. subtilis (wt) cells grown with 0.5 mM alkyne-containing EDA (panels (i) and (iv)), EDA-DA (panels (ii) and (v)), DA-EDA (panels (iii) and (vi)) for 5 min (panels (i)-(iii)) and 60 min (panels (iv)-(vi)). Panel (c) denotes control cell labeling with EDA. These aliquots together with unlabeled controls were ‘clicked’ to Alexa Fluor 488 azide and imaged. When the alkyne is on the N terminus (EDA-DA), labeling is comparable to EDA. On the other hand, the labeling with carboxy-terminal tag (DA-EDA) is much fainter.

FIG. 16B depicts an exemplary embodiment of phase contrast and epifluorescence microscopy of B. subtilis grown with 0.5 mM alkyne-containing EDA-DA or the L-enantiomer control ELA-LA for 45 min and clicked as described in FIG. 16A. The comparison of FIGS. 16A, B shows that the labeling is D-enantiomer specific. The partial lysis of the cells visible in phase contrast is caused by 70% ethanol fixation.

FIG. 16C depicts an exemplary embodiment of phase contrast and epifluorescence microscopy of live B. subtilis (wt) cells labeled with azide-containing ADA-DA (panel (i)) and DA-ADA (panel (ii)) at different concentrations (0.4 mM & 1.6 mM) for 60 min and clicked to Alexa Fluor 488 DIBO alkyne using a non-toxic procedure. The signals from N-terminally tagged dipeptide ADA-DA were much higher than the signal from DA-ADA labeled cells.

FIG. 16D depicts an exemplary embodiment of phase contrast and epifluorescence microscopy of live B. subtilis (ΔdacA) cells labeled with azide-containing DA-ADA at a single concentration (1.6 mM) for 60 min (panel (i)) or for 60 min with a 30 min chase (panel (ii)), and clicked as described in FIG. 16C.

FIG. 17 depicts an exemplary embodiment of phase contrast and epifluorescence microscopy of live S. pneumoniae cells labeled with azide-containing DA-ADA (panel (i)) and ADA-DA (panel (ii)) at a single concentration (0.5 mM) for 60 min and clicked as described in FIG. 16C. The signals from N-terminally tagged dipeptide ADA-DA were much higher than the signal from DA-ADA labeled cells.

FIG. 18 depicts an exemplary embodiment of phase contrast and epifluorescence microscopy of polarly growing S. venezuelae cells grown with the blue fluorescent D-amino acid HADA (2 h, 0.5 mM) for several generations and briefly pulsed with alkyne-containing EDA-DA (10 min, 0.5 mM) and clicked with Alexa Fluor 488 azide and imaged. The signal from EDA-DA complements the signal from HADA. This result shows that dipeptide probes label the cell wall at sites of new PG synthesis.

FIG. 19A depicts an exemplary embodiment of differential interference contrast microscopy of L2 cells infected for 18 h with C. trachomatis in the presence of the dipeptide probe EDA-DA (1 mM).

FIG. 19B depicts an exemplary embodiment of fluorescence microscopy of the L2 cells as in FIG. 19A following binding of the probe to an azide modified Alexa Fluor 488 (green) via click chemistry and incubation with an antibody to MOMP and DAPI staining. Antibody to MOMP (red) was used to label chlamydial EBs and RBs. DAPI (blue) was used for nuclear staining. The fluorescence microscopy was imaged following a merge of all three fluorescent channels. Boxes indicate location of chlamydial inclusions.

FIG. 19C depicts an exemplary embodiment of fluorescence microscopy of the magnification of one boxed region of FIG. 19B.

FIG. 19D depicts an exemplary embodiment of fluorescence microscopy of the magnification of one boxed region of FIG. 19B.

FIG. 19D depicts an exemplary embodiment of fluorescence microscopy of the magnification of one boxed region of FIG. 19B.

FIG. 20A depicts an exemplary embodiment of DIC microscopy (panel (i)), fluorescence microscopy (panels (ii)-(iv)) of C. trachomatis-infected L2 cells 18 h post infection in the presence of 1 mM EDA-DA and D-cycloserine (DCS) and following binding of the probe to an azide-modified Alexa Fluor 488 (green) via click chemistry (panel (ii)) and incubation with an antibody to MOMP and DAPI staining. Antibody to MOMP (red) was used to label chlamydial EBs and RBs (panel (iii)). DAPI (blue) was used for nuclear staining. A merge of all three fluorescent channels is presented in panel (iv). Fluorescent images are maximum intensity projections of z-stacks.

FIG. 20B depicts an exemplary embodiment of DIC microscopy (panel (i)), fluorescence microscopy (panels (ii)-(iv)) of C. trachomatis-infected L2 cells 18 h post infection in the presence of 1 mM EDA-DA and ampicillin (AMP) and following binding of the probe to an azide-modified Alexa Fluor 488 (green) via click chemistry (panel (ii)) and incubation with an antibody to MOMP and DAPI staining. Antibody to MOMP (red) was used to label chlamydial EBs and RBs (panel (iii)). DAPI (blue) was used for nuclear staining. A merge of all three fluorescent channels is presented in panel (iv). Fluorescent images are maximum intensity projections of z-stacks.

While the present invention is amenable to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments and claims herein for interpreting the scope of the invention.

### DETAILED DESCRIPTION

The compositions and methods now will be described more fully hereinafter with reference to the accompanying drawings, in which some, but not all permutations and variations of embodiments of the invention are shown. Indeed, the invention may be embodied in many different forms and should not be construed as limited to the embodiments set forth herein. These embodiments are provided in sufficient written detail to describe and enable one skilled in the art to make and use the invention, along with disclosure of the best mode for practicing the invention, as defined by the claims and equivalents thereof.

Likewise, many modifications and other embodiments of the compositions and methods described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

The following abbreviations are used throughout the disclosure: EDA, ethynyl-D-alanine; ELA, ethynyl-L-alanine, DA, D-alanine; LA, L-alanine; DMF, N,N-dimethylformamide; DCM, dichloromethane; DEPBT, 3-(Diethoxyphophoryloxy)-1,2,3-benzotriazin-4(3H)-one; TFA, trifluoroacetic acid; TMS, trimethylsilyl-; Bn, benzyl-; Boc, t-butoxycarbonyl-; DCC, dicyclohexylcarbodiimide; DMAP, 4-dimethylaminopyridine; MeCN, acetonitrile; HATU, 2-(7-Aza-1H-benzotriazole-1-yl)-1,1,3,3-tetramethyluronium hexafluorophosphate; DDQ, 2,3-dichloro-5,6-dicyano-1,4-benzoquinone; TMSE, trimethylsilylethyl-; EDA-DA, ethynyl-D-alanyl-D-alanine; ELA-LA, ethynyl-L-alanyl-L-alanine; DA-EDA, D-alanyl-ethynyl-D-alanine; ADA-DA, azido-D-alanyl-D-alanine; and DA-ADA, D-alanyl-azido-D-alanine.

### Overview

Previous efforts to label PG in live bacteria principally have relied upon cell wall-active antibiotics (e.g., vancomycin, ramoplanin) modified with fluorophores or cell wall precursors/substrates covalently modified with fluorescent reporter groups. The compositions and methods described herein, however, take advantage of mechanisms for incorporating labeled DAAs and dipeptides into the stem peptides displayed on a bacterial cell wall surface.

The work described herein demonstrates how to make derivatized DAA and dipeptides having a suitable label, such as an appropriate fluorophore and how such derivatized compounds can be visualized in live cells by fluorescence microscopy following the incorporation of the derivatized compounds into PG and thus the cell wall. In the range of physiologically relevant concentrations, the incorporated FDAAs do not appear to be toxic to bacteria. Unlike previous methods that employ covalently modified cell wall precursors, the methods described herein do not appear to adversely affect cell morphology. In addition, the methods described herein enable pulse-chase experiments that cannot be easily executed in the presence of fluorescently modified cell wall active drugs. Because the disclosed derivatized compounds have low or minimal toxicity to live cells, they are ideal markers to evaluate and screen microbiostatic or microbiotoxic compounds that do adversely affect microorganism growth and viability, such as studies directed to development of novel antibiotics.

Studies disclosed herein demonstrate that the compositions and methods are applicable to a wide array of Gram-positive and Gram-negative bacteria and provides significant utility for probing PG biosynthesis, cell wall morphogenesis and the response of the PG biosynthetic machinery to cell wall-active agents and/or cell wall-disrupting agents. The present disclosure therefore provides compositions and methods for studying bacterial cell wall PG biosynthesis and for discovering bacterial cell wall-acting and/or cell wall-disrupting agents.

### Compositions

Fluorescent D-Amino Acids (FDAAs)

Compositions of the invention include labeled D-amino acids (DAAs), especially fluorescent D-amino acids (FDAAs). As used herein, “amino acid” or “amino acid residue” are used interchangeably to mean a molecule containing a first, or alpha, carbon attached to an amine group, a carboxylic acid group and a side-chain that is specific to each amino acid. A natural amino acid can include conventional elements such as carbon, hydrogen, oxygen, nitrogen and sulfur. An amino acid may be a naturally occurring amino acid or artificially-created unnaturally occurring amino acid. Preferably, the amino acid is naturally occurring, and, unless otherwise limited, may encompass known analogues/synthetics of natural amino acids that can function in a similar manner as naturally occurring amino acids. With the exception of glycine, the natural amino acids all contain at least one chiral carbon atom. These amino acids therefore exist as pairs of stereoisomers (D- and L-isomers). Of particular interest herein are D-isomers or D-amino acids, particularly D-Ala, D-Asp, D-Cys, D-Glu and D-Lys, which are frequently found in the stem peptide of the PG unit.

It is well known in the art that amino acids within the same conservative group typically can substitute for one another without substantially affecting the function of a protein. For the purpose of the present disclosure, such conservative groups are set forth in Table 1 and are based preferably on shared properties, as readily appreciated to those skilled in the art. See also, Alberts et al., “Small molecules, energy, and biosynthesis” 56-57 In: Molecular Biology of the Cell (Garland Publishing Inc. 3^{rd} ed. 1994).

TABLE 1
Amino Acids and Their Conservative Substitutions.

| | Side | | | Preferred |
|---|---|---|---|---|
| | Chain | Side Chain | Hydropathy | Conservative |
| Residue | Polarity | pH | Index | Substitutions |
| Ala (A) | Non-polar | Neutral | 1.8 | Ser |
| Arg (R) | Polar | Basic (strongly) | −4.5 | Lys, Gln |
| Asn (N) | Polar | Neutral | −3.5 | Gln, His |
| Asp (D) | Polar | Acidic | −3.5 | Glu |
| Cys (C) | Non-polar | Neutral | 2.5 | Ser |
| Gln (Q) | Polar | Neutral | −3.5 | Asn, Lys |
| Glu (E) | Polar | Acidic | −3.5 | Asp |
| Gly (G) | Non-polar | Neutral | −0.4 | Pro |
| His (H) | Polar | Basic (weakly) | −3.2 | Asn, Gln |
| Ile (I) | Non-polar | Neutral | 4.5 | Leu, Val |
| Leu (L) | Non-polar | Neutral | 3.8 | Ile, Val |
| Lys (K) | Polar | Basic | −3.9 | Arg, Gln |
| Met (M) | Non-polar | Neutral | 1.9 | Leu, Ile |
| Phe (F) | Non-polar | Neutral | 2.8 | Met, Leu, Tyr |
| Pro (P) | Non-polar | Neutral | −1.6 | Gly |
| Ser (S) | Polar | Neutral | −0.8 | Thr |
| Thr (T) | Polar | Neutral | −0.7 | Ser |
| Trp (W) | Non-polar | Neutral | −0.9 | Tyr |
| Tyr (Y) | Polar | Neutral | −1.3 | Trp, Phe |
| Val (V) | Non-polar | Neutral | 4.2 | Ile, Leu |

The following six groups each contain amino acids that are typical but not necessarily exclusive conservative substitutions for one another: 1) Alanine (A), Serine (S) and Threonine (T); 2) Aspartic acid (D) and Glutamic acid (E); 3) Asparagine (N) and Glutamine (Q); 4) Arginine (R) and Lysine (K); 5) Isoleucine (I), Leucine (L), Methionine (M), and Valine (V); and 6) Phenylalanine (F), Tyrosine (Y) and Tryptophan (W).

Examples of suitable labels for the DAAs include, but are not limited to, radiolabels, biotin (which may be detected by avidin or streptavidin conjugated to peroxidase), lanthanides, alkaline phosphatase and fluorescent labels (e.g., coumarins, fluoresceins, cyanines, bodipy dyes, green fluorescent protein, quantum dots rhodamine, especially the Alexa Fluor® family of fluorescent dyes available from Invitrogen/Molecular Probes). Other labels amenable for use in the modified D-amino acids disclosed herein include metals and isotopic labels.

Labeling of DAAs can be carried out by covalently attaching the label to a free amine group, such as free amine groups present on the side-chain that is specific to each amino acid. If the side chain lacks a free amine group, one of skill in the art understands how to add such groups, as is the case of adding such a group to D-Ala to obtain 3-amino-D-Ala. Some labels can be detected by using a labeled counter suitable for the detection of the label in question. In the Examples below, 7-hydroxycoumarin 3-carboxylic acid (HCC-OH), 7-nitrobenzofurazan (NBD), 4-chloro-7-nitrobenzofurazan (NBD-Cl), fluorescein (F) and carboxytetramethylrhodamine (T) were covalently attached to DAAs as labels.

Other coupling chemistries are known in the art that can be used for introducing labels into amino acids having functional groups other than an amine. Such amino acids include a functional alcohol group (e.g., serine and tyrosine), thiol group (e.g., cysteine), or carbonyl or carboxylate group (e.g., aspartate and glutamate). Such functional groups can be derivatized or reacted with suitably modified, activated coupling agents having labels of the types disclosed herein.

An example of a FDAA includes HADA, which is a HCC-OH-labeled 3-amino-D-Ala. Another example of a FDAA includes NADA, which is a NBD-Cl-labeled 3-amino-D-Ala. Another example includes FDL, which is a F-labeled D-Lys. Another example is TDL, which is a T-labeled D-Lys. Another example includes HDL, which is a HCC-OH-labeled D-Lys. Another example includes NDL, which is a NBD-Cl-labeled D-Lys. Another example includes FADA, which is a F-labeled 3-amino-D-Ala. Another example includes TADA, which is a T-labeled 3-amino-D-Ala. Other FDAAs can include a D-Glu having its side chain modified to include a free amine group linked to any of the fluorescent labels above (e.g., HADG, NADG, FADG and TADG).

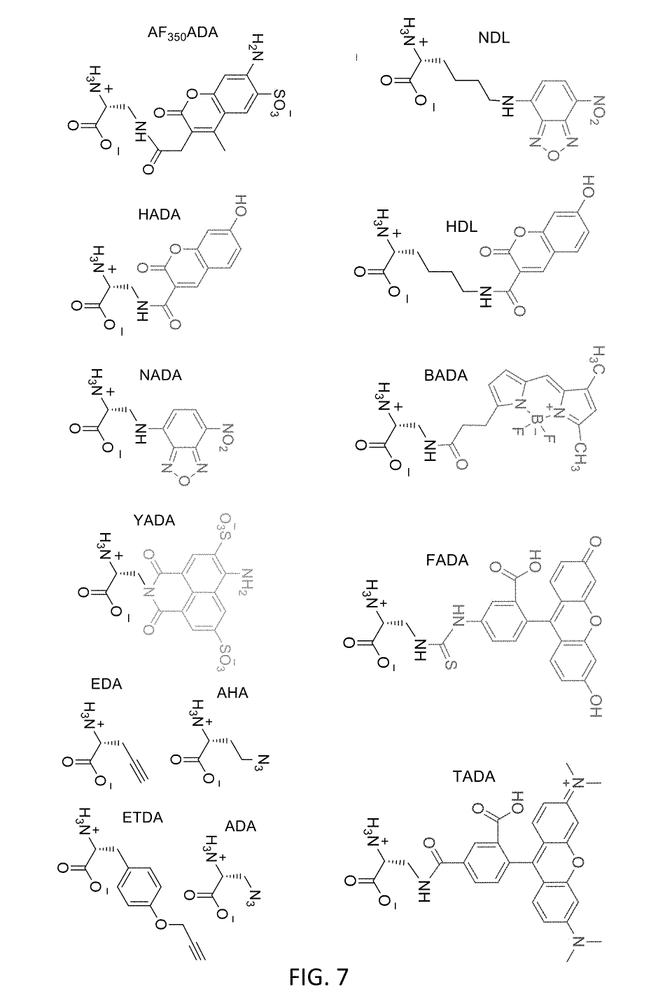

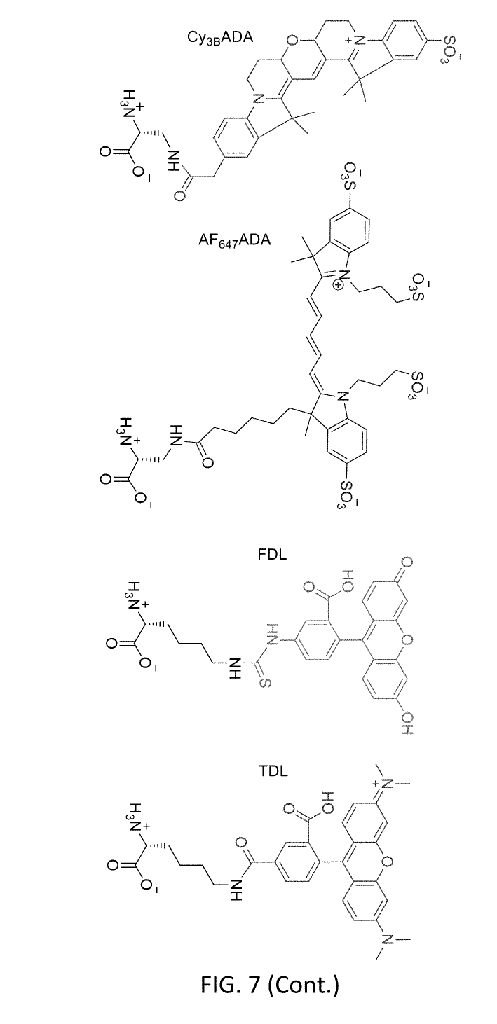

See, e.g., FIG. 7, for other examples of preferred labels and modified FDAAs.

Methods of fluorescently labeling and detecting amino acids are well known in the art. See, Braun & Dittrich (2010) Beilstein J. Org. Chem. 6:69; Katritzky & Narindoshvili (2009) Org. Biomol. Chem. 7:627-634; Merkel et al. (2010) Chembiochem. 11:305-314; Cava et al. (2011) Cell Mol. Life Sci. 68:817-831; Lam et al. (2009) Science 325:1552-1555; Cava et al. (2011) EMBO J. 30:3442-3453; and Lupoli et al. (2011) J. Am. Chem. Soc. 133:10748-10751.

Clickable D-Amino Acids (CDAA's)

Compositions of the invention also include clickable D-amino acids (CDAAs). The CDAAs have a DAA backbone that includes, for example, an alkyne or azide functional group present on the side-chain that is specific to each amino acid that can be captured in situ by a labeled, detecting agent carrying a conjugate functional group via click-chemistry. Functional groups in a DAA backbone that can be targeted by the labeled, detecting agent include, but are not limited to, primary amines, carboxyls, sulfhydryls, carbohydrates and carboxylic acids.

One of skill in the art is familiar with “click” chemistry, which utilizes chemical cross-linking agents to add functional groups to molecules. See, Kolb et al. (2001) Angew. Chem. Int. Ed. 40:2004-2021; and Evans (2007) Aust. J. Chem. 60:384-395.

Cross-linking and enrichment strategies for separating a cross-linking reaction from enrichment steps have been developed based on bioorthogonal chemistries including the azide-alkyne “click” cycloaddition and Staudinger ligation using alkyne- or azide-labeled cross-linking agents (e.g., fluorescent labels). Azides and alkynes are not naturally found in proteins, peptides, nucleic acids or glycans; therefore, these moieties can be engineered onto the DAAs and labeled, detecting agent to generate azide-containing molecules and alkyne-containing molecules that are reactive with one another. As such, the orthogonality of azides and alkynes to biological processes (e.g., competing reactions) is a significant advantage of these methods. Moreover, “click” cycloadditions can be performed under aqueous conditions, allowing enrichment by conjugation of an appropriate affinity or labeling tag. See, generally, Rostovtsev et al. (2002) Angew. Chem. Int. Ed. 41:2596-2599; Tornoe et al. (2002) J. Org. Chem. 67:3057-3064; Baskin et al. (2007) Proc. Natl. Acad. Sci. USA 104:16793-16797; Saxon et al. (2000) Science 287:2007-2010; Chowdhury et al. (2009) Anal. Chem. 81:5524-5532; Trnka & Burlingame (2010) Mol. Cell. Proteomics 9:2306-2317; Nessen et al. (2009) J. Proteome Res. 8:3702-3711; Vellucci et al. (2010) J. Am. Soc. Mass Spectrom. 21:1432-1445; and Jewett & Bertozzi (2010) Chem. Soc. Rev. 39:1272-1279. See also, Int'l Patent Application Publication No. WO 2012/006603. As used herein, “click chemistry” and “clickable” therefore mean a reaction between azide-containing molecules and alkyne-containing molecules to yield a covalent product-1,5-disubstituted 1,2,3-triazole. The reaction can be a copper(I)-catalyzed alkyne azide cycloaddition (CuAAC) or, in cases where copper toxicity may be an issue, can be a copper(I)-free-catalyzed alkyne azide cycloaddition.

Examples of functional groups for use on azide-containing molecules and alkyne-containing molecules include, but are not limited to, hexynyl groups, pentynyl groups, heptynyl groups, azido-propyl groups, azido-butyl groups and azido-pentyl groups. When the functional group of alkyne-containing molecules (e.g., DAAs) is an alkynyl group (e.g., hexynyl, pentynyl or heptynyl), the functional group of azide-containing molecules (e.g., labeled, detecting agents) has the corresponding clickable azido group. Likewise, when the functional group of azide-containing molecules (e.g., DAAs) is an azide group (e.g., azido-propyl, azido-butyl, or azido-pentyl), the functional group of alkyne-containing molecules (e.g., labeled, detecting agents) has the corresponding clickable alkynyl group.

As such, various labeling designs for detecting agents are known including, but not limited to, biotinylated agents, isotope-coded agents, fluorophore-labeled agents, mass-tag-labeled agents and chromophore-labeled agents. It is also known that the addition of functional groups can cause the cross-linker to become very bulky or less cell-permeable, and thus not very effective for in vivo and/or in situ cross-linking. To reduce the total size of the cross-linker, separation of the cross-linking step from conjugation of affinity tags can be one effective strategy. See, Trester-Zedlitz et al. (2003) J. Am. Chem. Soc. 125:2416-2425; Tang et al. (2005) Anal. Chem. 77:311; Kang et al. (2009) Rapid Commun. Mass Spectrom. 23:1719-1726; Chu et al. (2006) J. Am. Chem. Soc. 128:10362-10636; Muller et al. (2001) Anal. Chem. 73:1927-1934; Collins et al. (2003) Bioorg. Med. Chem. Lett. 13:4023-4026; Petrotchenko et al. (2005) Mol. Cell. Proteomics 4:1167-1179; Wine et al. (2002) Anal. Chem. 74:1939-1945; Sinz et al. (2001) Biochemistry 40:7903-7913; and Sinz & Wang (2004) Anal. Biochem. 331:27-32.

In some instances, the detecting agents, whether having an alkynyl or azido functional group, can be fluorophore-labeled. Examples of fluorophores include, but are not limited to, Alexa Fluor® dyes, BODIPY® dyes, fluorescein, Oregon Green® 488 and Oregon Green® 514 dyes, Rhodamine Green and Rhodamine Green-X dyes, eosin, tetramethylrhodamine, Lissamine Rhodamine B and Rhodamine Red-X dyes, X-Rhodamine, Texas Red® and Texas Red®-X dyes, naphthofluorescein, Carboxyrhodamine 6G, QSY dyes:fluorescence quenchers, nonfluorescent malachite green, coumarin derivatives, Pacific Orange dye, cascade blue and other pyrene derivatives, cascade yellow and other pyridyloxazole derivatives, naphthalenes (e.g., dansyl chloride), dapoxyl dye, bimane, 1-dimethylamine-N(2-azido-ethyl) naphthalene-5-sulfonamide, 6-(6-amino-2-(2-azidoethyl)1,3-dioxo-1H-benzo(de)-2(3H)isoquinoline, 6-(6-amino-2-(2-propinyl)1,3-dioxo-1H-benzo(de)-2(3H)isoquinoline, 8-(4-azidoethyloxyphenyl)-2,6-diethyl-1,3,5,7-tetramethyl-4,4-difluoro-4-bora-3a,4a-diaza-s-indacene, 8-(4-propynyloxyphenyl)-2,6-diethyl-1,3,5,7-tetramethyl-4,4-difluoro-4-bora-3a,4a-diaza-s-indacene, 1-(3-azido-propoxy)-7-methylamino-phenoxazin-3-one, 1-(2-propynyl)-7-methylamino-phenoxazin-3-one, N-(5-(3-azidopropylamino)-9H-benzo(a)-phenoxazin-9-ylidene)-N-methyl-methanaminium chloride, N-(5-(3-propynyl-amino)-9H-benzo(a)-phenoxazin-9-ylidene)-N-methyl-methanaminium chloride, (9-(3-azido-propoxy)-7-piperidin-1-yl-phenoxazin-3-ylidene)-dimethyl-ammonium perchlorate. See also, Kele et al. (2009) Org. Biomol. Chem. 7:3486-3490; Nagy et al. (2010) Chem. Asian J. 5:773-777; Filnov et al. (2011) Nat. Biotechnol. 29:757-761; Subach et al. (2011) Nature Methods 8:771-777; Yang et al. (2011) J. Am. Chem. Soc. 133:9964-9967; Zin (2011) Nature Methods 8:726-728; and “Fluorophores and their amine-reactive derivatives,” Chapter 1 and “Click Chemistry and other functional group modifications,” Chapter 3 in The Molecular Probes® Handbook (available on the World Wide Web at invitrogen.com/site/us/en/home/References/Molecular-Probes-The-Handbook.html). A variety of clickable fluorophores are commercially available from, for example Sigma Aldrich, Active Motif Chromeon and Invitrogen/Molecular Probes. In the examples below, CDAAs were clicked with red sulfo-Cy3-azide.

An example of a CDAA includes EDA. Another example of a CDAA includes ADA. See, e.g., FIG. 7.

Fluorescent Muramylpentapeptide Precursor Units (FMPUs)

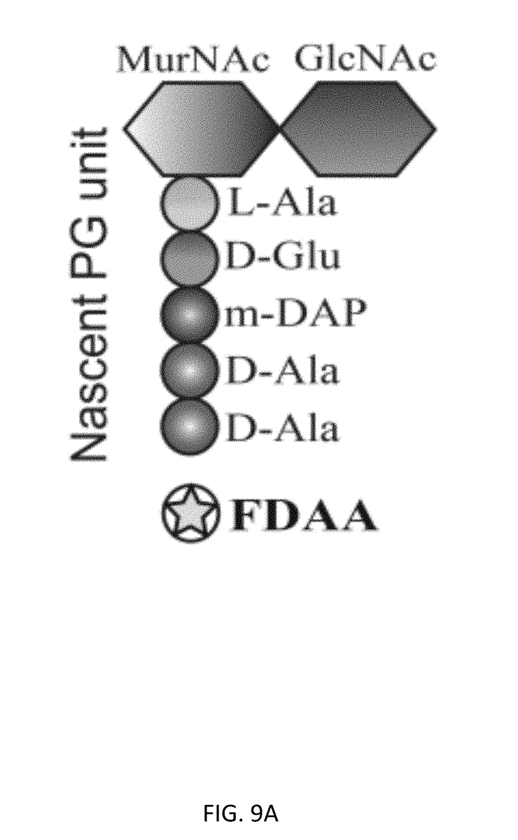

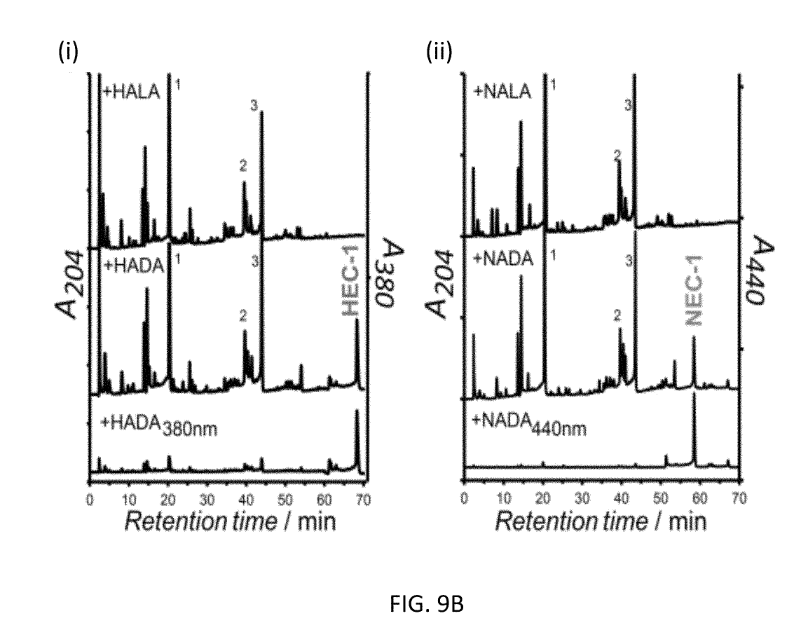

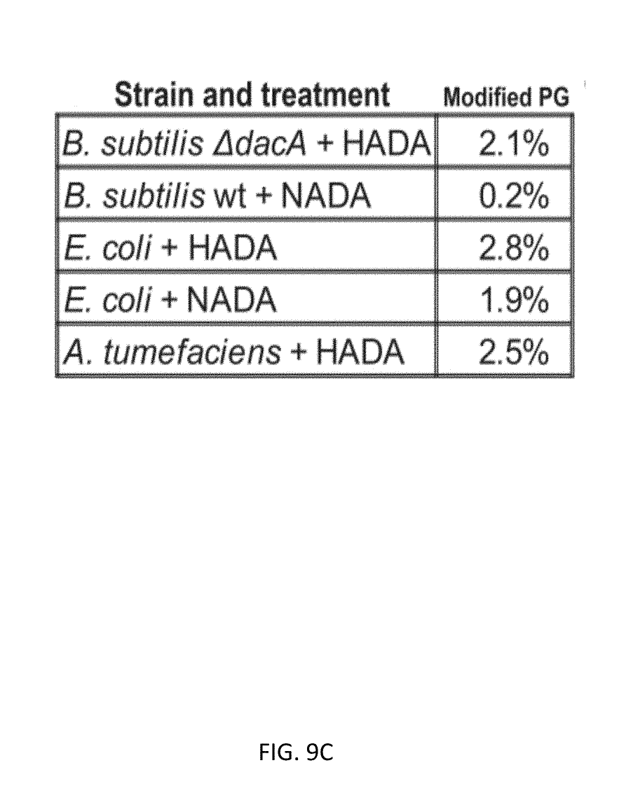

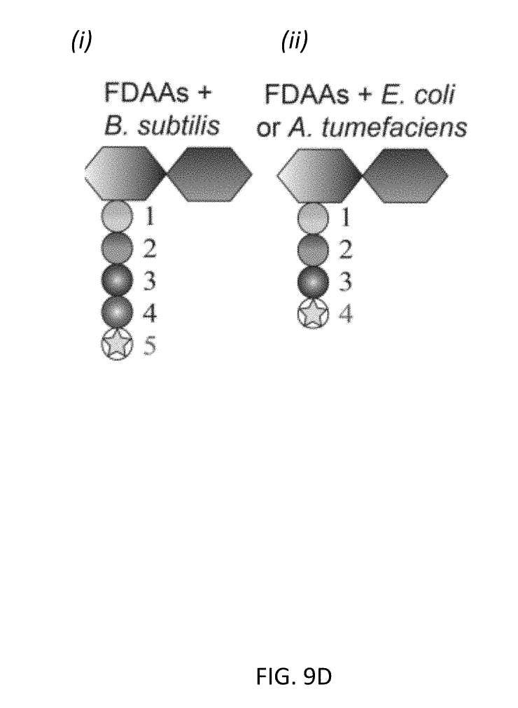

Compositions of the invention also include fluorescent muramylpentapeptide precursor units (FMPUs) having an NAM moiety with a peptide chain of three to five amino acids in which one or more of the amino acids in the stem peptide are FDAAs and/or CDAAs as described herein. See, e.g., FIGS. 9A and 9D.

Fluorescent Peptidoglycan Units (FPGUs)

Compositions of the invention also include fluorescent peptidoglycan units (FPGUs). The FPGUs have a FMPU as described herein linked to a NAG moiety. See, e.g., FIGS. 9A and 9D.

Bacterial Cells Having Fluorescent D-Amino Acids

Compositions of the invention also include live bacteria having FDAAs, CDAAs, FMPUs and/or FPGUs as described herein incorporated into PG in a cell wall.

While Gram-positive bacteria tend to have a thicker PG layer, it is intended that the bacteria can be Gram-positive bacteria or Gram-negative bacteria. Examples of suitable Gram-positive bacteria include, but are not limited to, Actinomyces spp., Bacillus spp., Brachybacterium spp., Clostridium spp., Corynebacterium spp., Diplococcus spp., Enterococcus spp., Lactococcus spp., Listeria spp., Nocardia spp., Propionibacterium spp., Staphylococcus spp., Streptococcus spp. Streptomyces spp. In the examples below, live B. subtilis, B. conglomeratum, L. lactis, S. aureus, S. pneumoniae, S. venezuelae, were grown in the presence of FDAAs and/or CDAAs.

Examples of suitable Gram-negative bacteria include, but are not limited to, Acinetobacter spp., Agrobacterium spp., Bordetella spp., Borrelia spp., Brucella spp., Burkholderia spp., Campylobacter spp., Caulobacter spp., Chlamydia spp., Enterobacter spp., Escherichia spp., Helicobacter spp., Hemophilus spp., Klebsiella spp., Legionella spp., Neisseria spp., Proteus spp., Pseudomonas spp, Salmonella spp., Shigella spp., Synechocystis spp., Verrucomicrobia spp., Vibrio spp. and Yersina spp. In the examples below, live A. tumefaciens, B. phytofirmans, C. crescentus, E. coli, Synechocystis sp. PCC 6803 and V. spinosum were grown in the presence of FDAAs and/or CDAAs.

### Kits

Compositions of the invention also include kits having one or more FDAA, CDAA, FMPU and/or FPGU as described herein and optionally one or more labeled detecting agents (if CDAAs are included in the kits) for use in in situ labeling/probing of PG during biosynthesis, as well as for screening for bacterial cell wall-acting and/or cell wall-disrupting agents. The kits also can include additional reagents such as unlabeled DAAs, unlabeled L-amino acids (LAAs) and/or labeled LAAs. The kits also can include positive and/or negative bacterial controls, where the controls have unlabeled DAAs, CDAAs and LAAs or labeled DAAs and LAAs incorporated into PG in a cell wall.

As used herein, “kit” means any manufacture (e.g., a package or a container) having, for example, at least one FDAA and/or CDAA and a positive and/or negative control. The kit may be promoted, distributed, or sold as a unit for performing any of the methods described herein.

Though not necessarily required, kits preferably include instructions, procedures and/or directions that guide users or ones skilled in the art how to use the agents, reagents, and/or other components for their intended purpose. For example, kits can include a package insert describing procedures for carrying out any one of the methods described herein or analytical information for correlating the level of expression measured in live bacteria. Likewise, the package insert can include representative images of positive or negative samples with low or high levels of incorporation as compared to an appropriate control. The kits can be promoted, distributed or sold as units for performing the methods described below.

The kits also can include a receptacle or other means for holding a sample to be evaluated for FDAA and/or CDAA incorporation, and means for determining the presence and/or quantity of FDAA and/or CDAA incorporation in live bacteria.

The kits also can include at least one buffer. Examples of buffers include, but are not limited to, cell isolation buffers, fixation buffers, lysis buffers, permeabilization buffers, sonication buffers, separation buffers, stabilization buffers and wash buffers. Though not limited, buffers include strong acids in combination with weak bases, strong bases in combination with weak acids, a combination of weak acids and bases, or even a small or low concentration (e.g., within the range from about 0.1 mM to about 10 mM) of an acid or base, in the absence of a conventional conjugate base or acid, respectively; typically, however another component of the mixture may provide such conjugate acid or base function. Examples of acids and bases, both in terms of ionization/dissociation strength (i.e., strong or weak) and type (i.e., inorganic or organic), are well known in the art.

Any or all of the kit components can be provided within containers that protect them from the external environment, such as in sealed containers.

### Methods

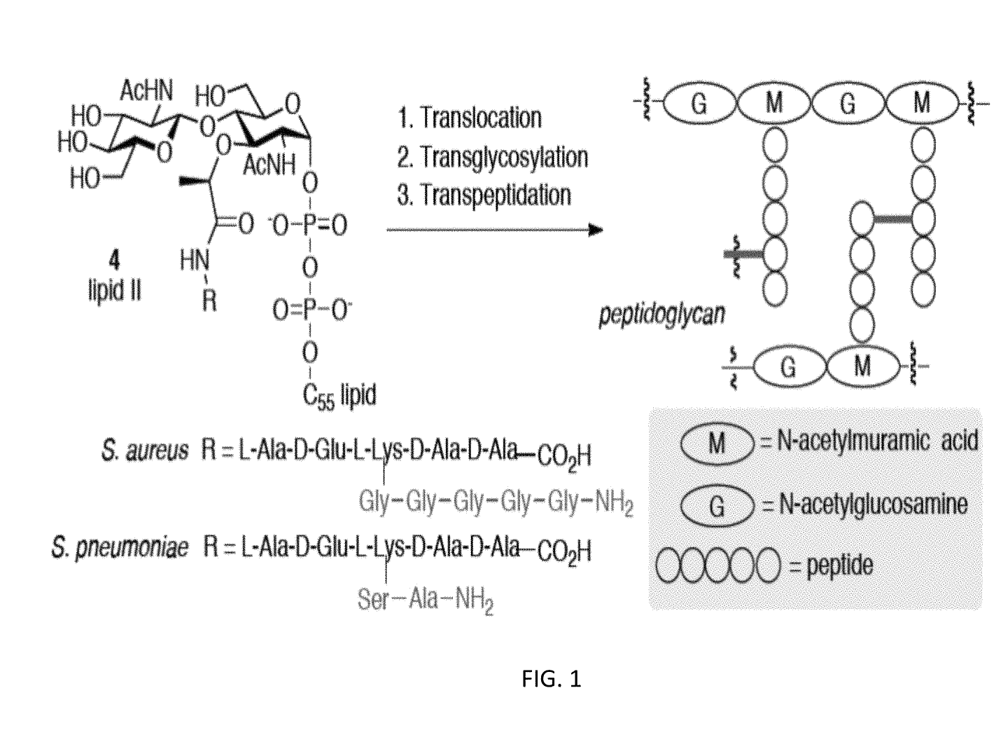

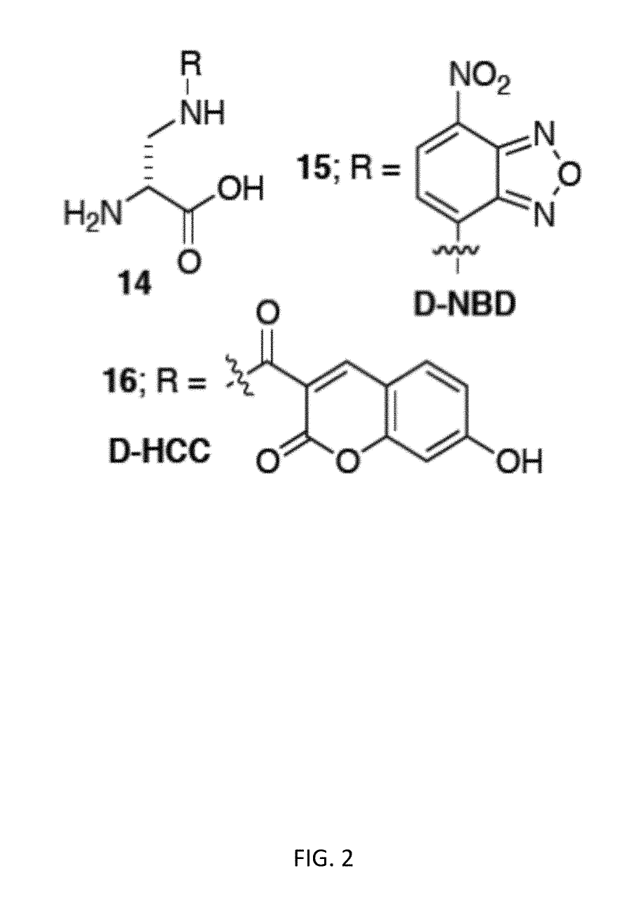

Methods include assessing bacterial cell wall biosynthesis (and PG recycling) in real time. As shown in FIG. 1, bacterial cell wall biosynthesis typically involves three steps: translocation, transglycosylation and transpeptidation. In the translocation and transglycosylation steps, carbohydrate backbone is formed by polymerization via glycosidic bond formation between the C(4)-hydroxyl of a membrane-bound lipid II intermediate and the anomeric center of a membrane-bound glycan strand. Bacterial transpeptidases mediate crosslinking of the resulting elongated glycan strand. The cross-link is installed via attack of an amino group, either from the Lys residue itself or from a short peptide chain appended to the Lys residue, onto the penultimate D-Ala residue of an adjacent pentapeptide strand and results in cleavage of the terminal D-Ala residue. This rigid macromolecular structure, essential to both Gram-negative and Gram-positive bacteria, enables bacterial cells to resist lysis and, subsequently, cell death resulting from high internal osmotic pressure.

These methods typically begin by providing live Gram-positive or Gram-negative bacteria with FDAAs and/or CDAAs as described herein under conditions where the bacteria can covalently incorporate the FDAAs and/or CDAAs into PG of a bacterial cell wall. The FDAAs and/or CDAAs can be provided to organisms preferably within a given range of concentrations, for example, from about 0.1 μM to about 1 mM, as well as in any whole integer or fractional integer concentration thereof within this preferred range. The FDAAs and/or CDAAs can also be provided to organisms at preferred concentrations, for example, at about 0.1 μM and about 1 mM. Other ranges are also possible besides this preferred range and fall within the scope of this disclosure, the specific identification of which depends upon the particular biological organism or system under study, as well as upon the nature of the FDAAs and/or CDAAs used, their physiochemical properties and uptake by the particular biological organism or system under study, as well as the experimental set-up and purpose of the study at hand, as one of skill in the art would understand.

Determination of the optimal concentration (or amount) of FDAAs and/or CDAAs and the preferred ranges thereof for a particular organism is the subject of routine experimentation well within the purview of those skilled in the art. A typical route to ascertaining the optimal concentrations and preferred ranges of the FDAAs and/or CDAAs described herein is to perform a dose response experiment, wherein the parallel populations of a given organism are contacted with different concentrations (or amounts) of a given FDAA and/or CDAA, and the extent of incorporation of the compound(s) is assessed by biochemical assay (e.g., extent of compound labeling in PG fractions) and/or by visualization methods (e.g., fluorescence microscopy). Other approaches to selecting the optimal concentration (of amount) of FDAAs and/or CDAAs and the preferred ranges thereof for a particular organism are viable as well, as one skilled in the art would readily appreciate based upon this disclosure.

The methods also can include detecting the FDAAs and/or CDAAs in the bacterial cell wall to verify that they have been incorporated. The FDAAs and/or CDAAs (after being clicked) can be detected via fluorescence microscopy and other methods, depending upon the type of label or reporter used.

### Screening Methodologies

The cell wall biosynthetic pathway is unique to bacterial cells; therefore, agents that inhibit steps within this pathway are anticipated to show selective toxicity toward bacterial cells. As such, methods of the invention also can include screening for putative cell wall-acting or cell wall-disrupting agents. As used herein, “cell wall-acting” means an ability of an agent to interfere with PG biosynthesis in a bacterial cell wall, especially at the transglycosylation step, as this step takes place on the outer leaflet of the cell membrane so cellular penetration is not a prerequisite for the agent to manifest its biological activity. As used herein, “cell wall-disrupting” means an ability of an agent to disrupt or weaken the integrity of PG in an existing bacterial cell wall.

The methods can begin by contacting bacteria with a putative cell wall-acting agent or putative cell wall-disrupting agent, where the agent is cell wall-acting if the agent interferes with ongoing peptidoglycan biosynthesis in a bacterial cell wall or is cell wall-disrupting if the agent weakens integrity of peptidoglycan in an existing bacterial cell wall. When screening for putative cell wall-acting agents, the bacteria can be co-contacted with FDAAs and/or CDAAs as described herein simultaneously with the putative agent. When screening for putative cell wall-disrupting agents, the bacteria can have FDAAs and/or CDAAs as described herein covalently incorporated into PG of the cell wall prior to being contacted with the putative agent.

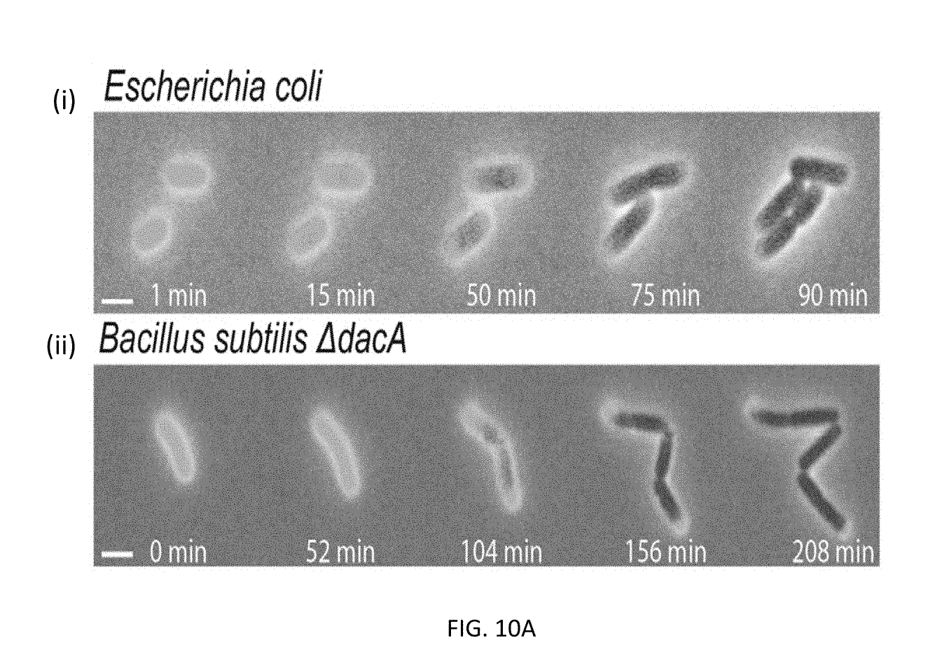

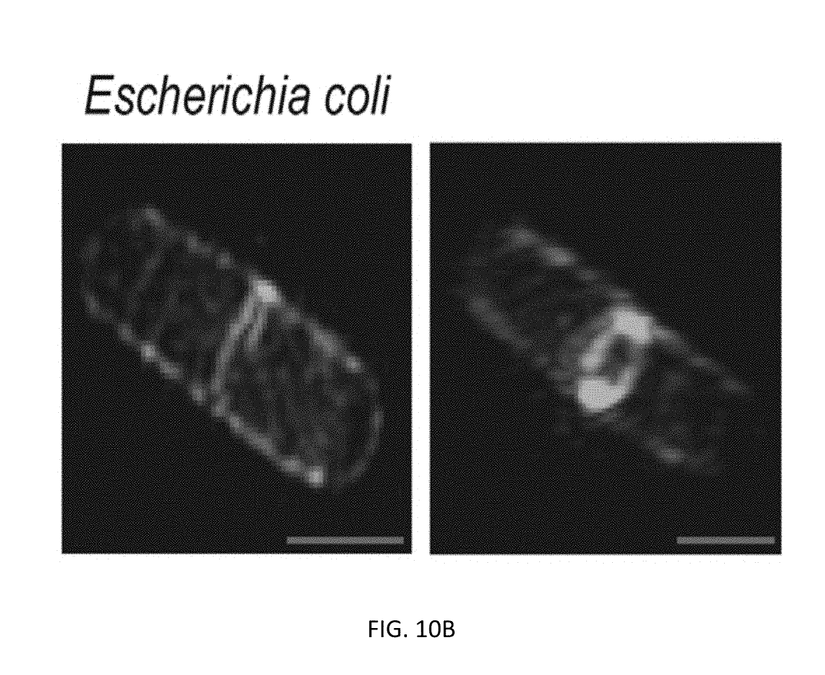

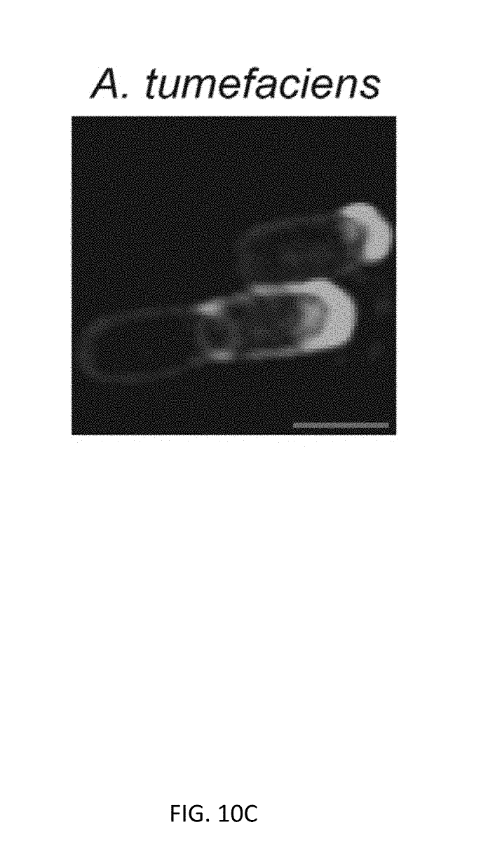

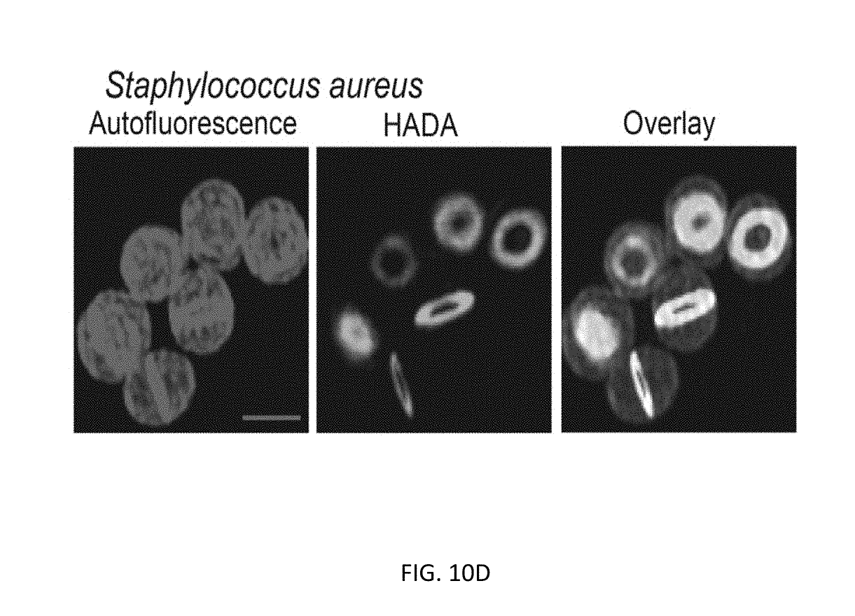

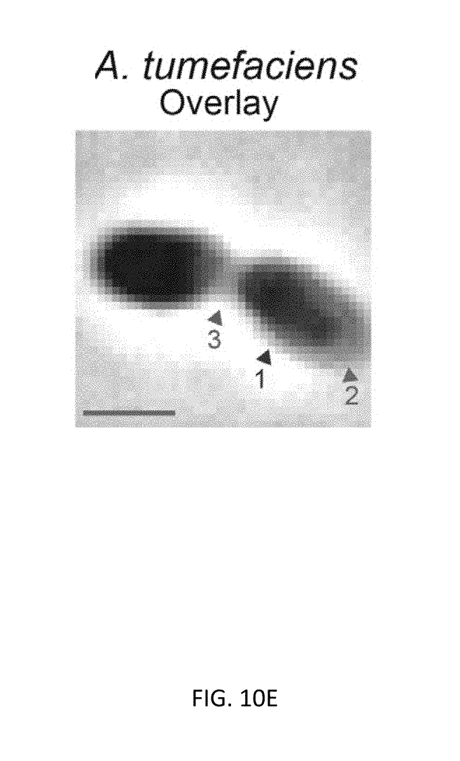

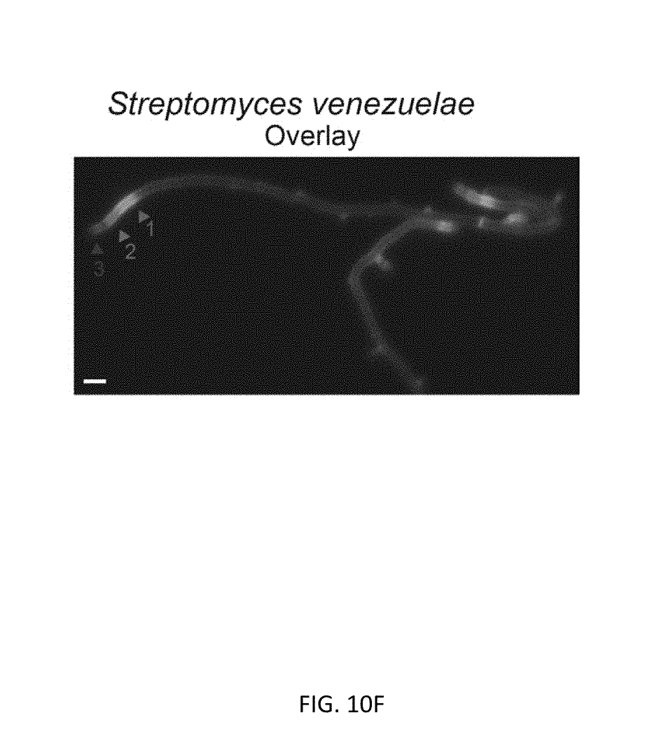

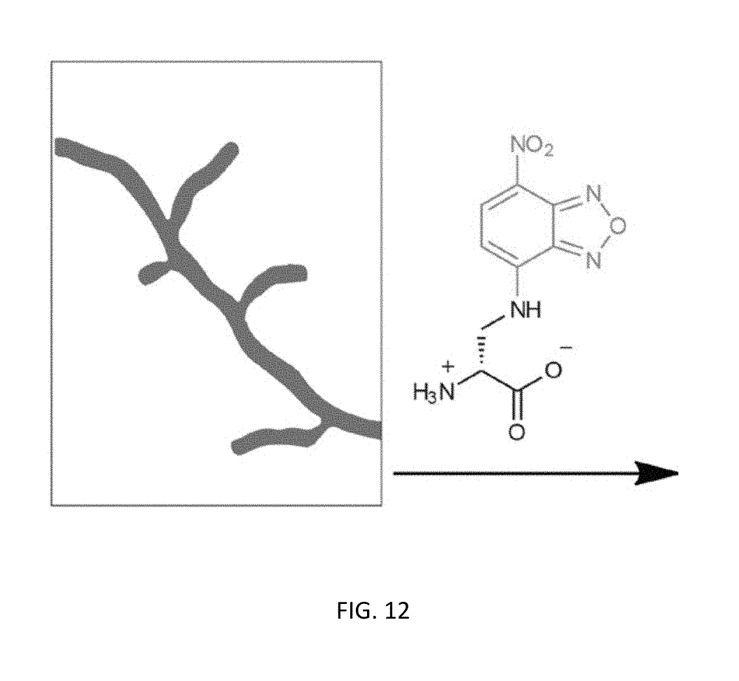

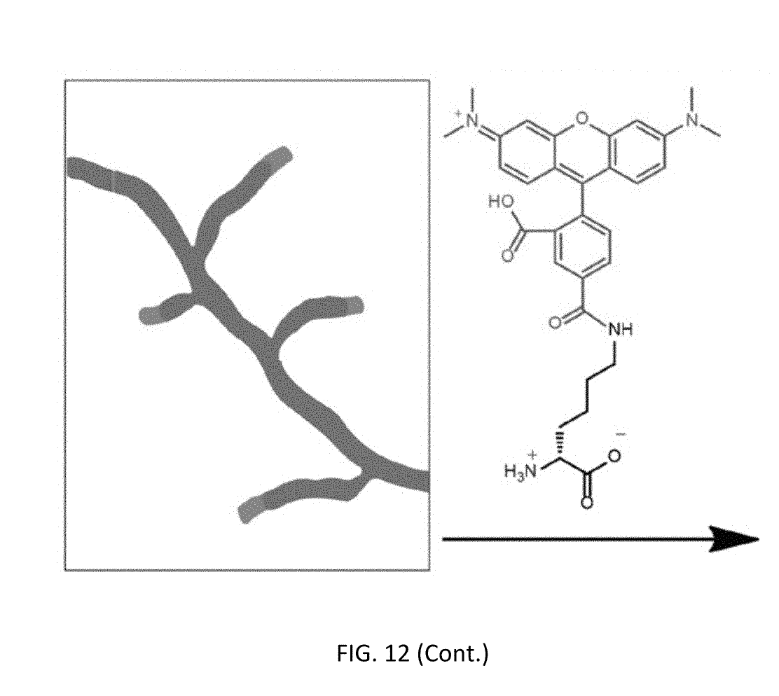

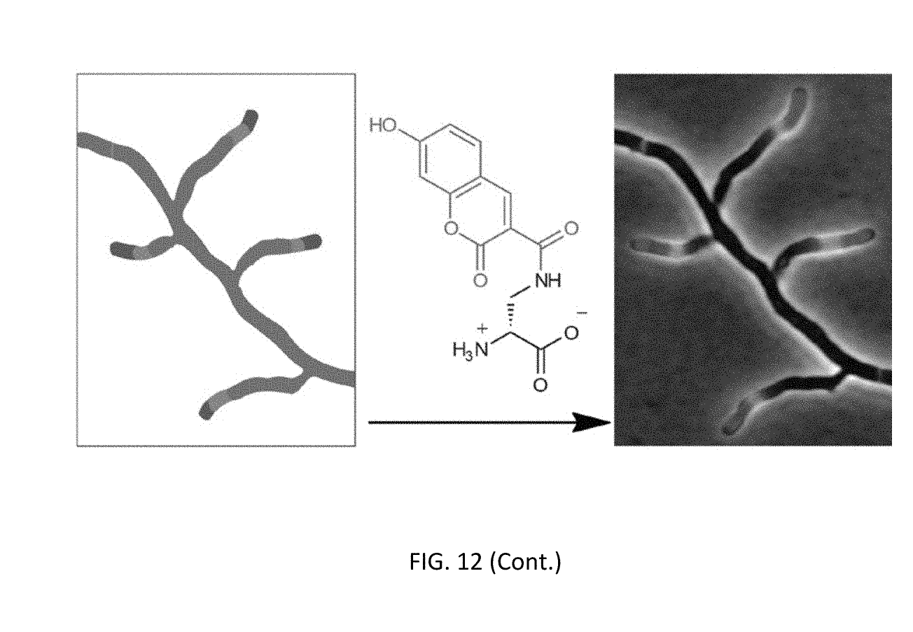

The methods also can include detecting whether the FDAAs and/or CDAAs have been incorporated in the bacterial cell wall or whether the FDAAs and/or CDAAs remain in the bacterial cell wall. As noted above, the FDAAs and/or CDAAs (after being clicked) can be detected via fluorescence microscopy and other methods, depending upon the type of label or reporter used. The pattern and/or location of FDAAs and/or CDAAs incorporation can be used to identify the bacteria (see, e.g., FIGS. 10-12).

The methods also can include comparing the results from the putative cell wall-acting agent or cell wall-disrupting agent with a known cell wall-acting agent or known cell wall-disrupting agent.

The compounds of the present disclosure have utility for identifying bacteria. As demonstrated in the Examples set forth herein, certain bacterial species display unique specificity for incorporating certain D-amino acids in PG and the bacteria cell wall. Thus, the use of the disclosed modified D-amino acids of the present disclosure enable identification of bacterial species by virtue of the pattern of labeling observed in the bacteria as a result of incorporation of the modified D-amino acids into PG of the bacterial cell wall.

A-Ala-D-Ala-Based Dipeptides as Tools for Imaging Peptidoglycan Biosynthesis

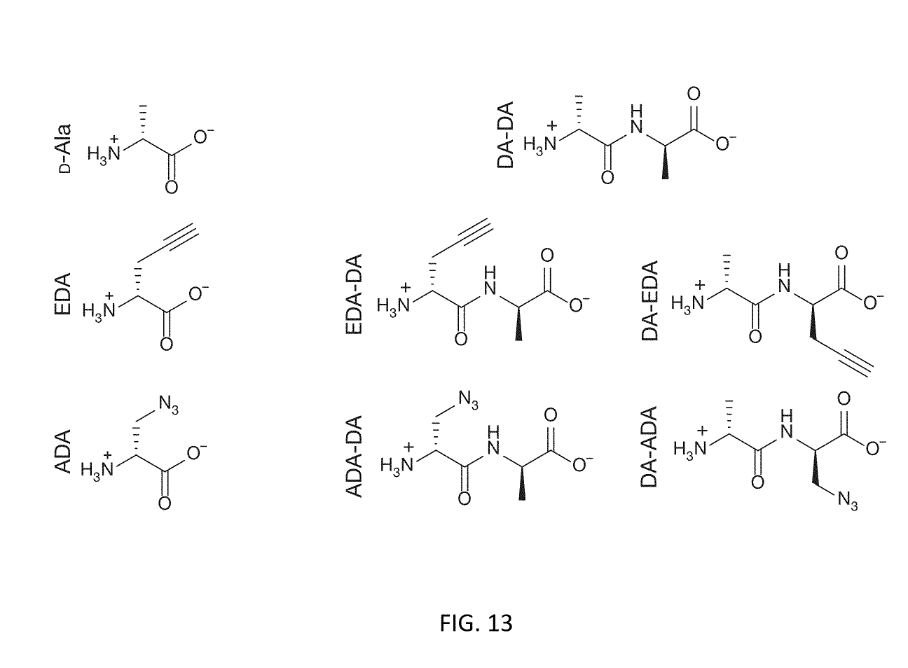

Referring to FIG. 13, the use of modified dipeptides, such as D-Ala-D-Ala (DA-DA) is described, wherein one of the D-Ala (DA) residues is replaced by azido-D-alanine (ADA) or ethynyl-D-alanine (EDA). The azido or ethynyl functional group provides a handle for capture by a probe substrate, containing a complementary functional group, via click chemistry.

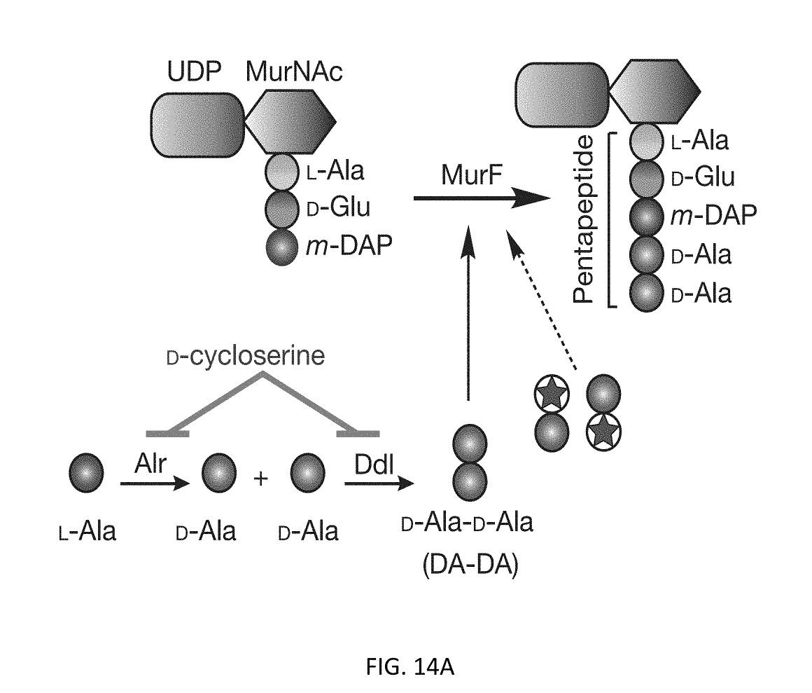

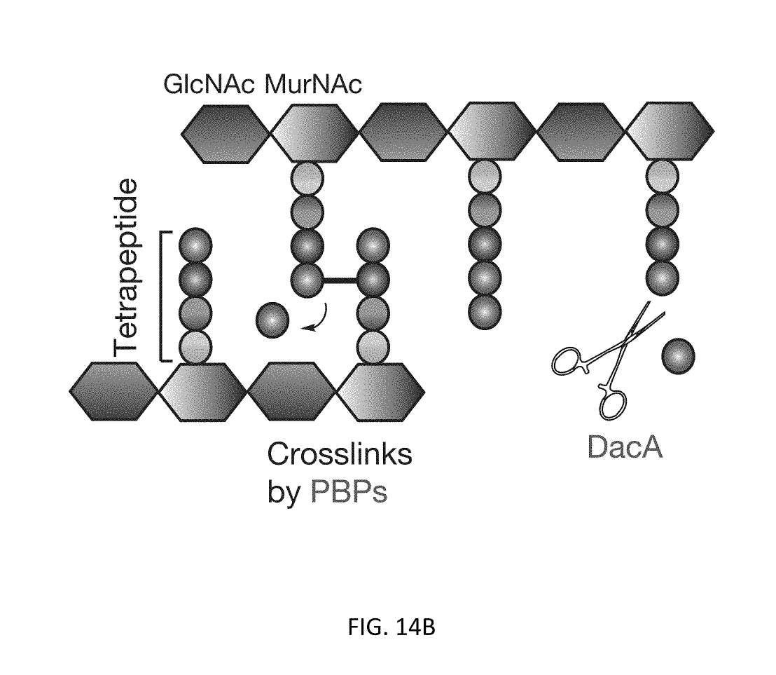

The dipeptide PG labeling strategy is shown in FIG. 14. Upon exposure of the modified dipeptide (DA-EDA, DA-ADA, EDA-DA, or ADA-DA; see FIG. 13 for chemical structures) to bacterial cells, the dipeptide is taken up by the cells and incorporated into precursors required for synthesis of peptidoglycan (FIG. 14A, B). After incorporation into peptidoglycan, the azido or ethynyl functional group is displayed on the cell surface where it can be readily captured as described above. This finding is significant in that it provides an alternative to prior methods that utilizes fluorescently modified D-amino acids (FDAAs). Additionally, the mode of dipeptide incorporation is fundamentally different than the mode(s) of incorporation utilized for the FDAAs.

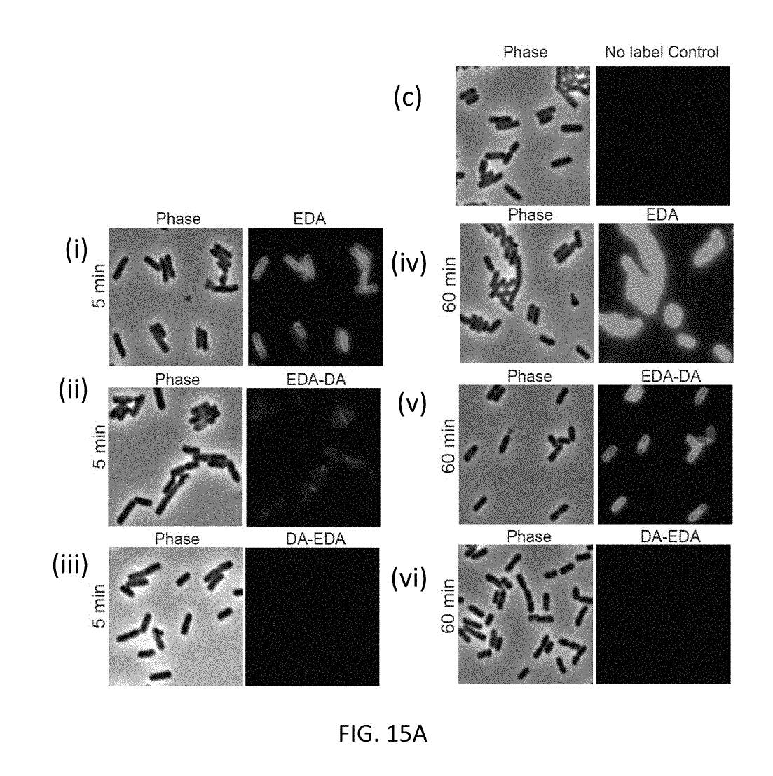

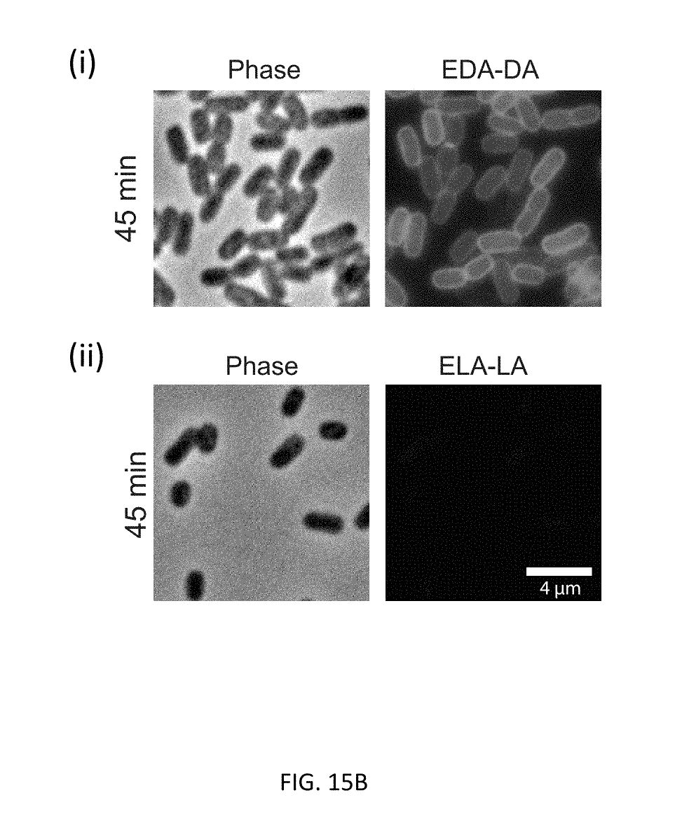

The DA-DA analogs modified with small bioorthogonal tags can be incorporated into growing peptide chains of PG in any bacteria (FIGS. 15-19). Once on PG, these tags can be selectively captured by any molecule carrying the conjugate functional group via click chemistry. When the Gram negative model Escherichia coli and evolutionary distant Gram positive model Bacillus subtilis cells are depleted of the essential intracellular DA-DA pools either by D-cycloserine (DCS), an inhibitor of bacterial alanine racemase and D-alanine ligase, or in E. coli by knocking-out the D-alanine ligase function, the growth can be rescued by the exogenously provided DA-DA and/or its labeled analogs (see Table 2). This indicates that the labeled DA-DA analogs can either completely (with DA-EDA or DA-ADA) or partially (with EDA-DA or ADA-DA) take over the function of intracellular DA-DA and covalently tag the PG by hijacking the MurF enzyme through the cytoplasmic precursor Park nucleotide (Table 2).

TABLE 2
Results of rescue assays for bacterial strains grown in different media

| | E. coli | | | |
|---|---|---|---|---|
| | Δddla, | | | |
| Condition | Δddlb | Condition | E. coli wt | B. subtilis wt |
| MM¹ | − | MM | +++++ | +++++ |
| MM + DA-DA | ++ | MM + DCS (20 μM for | − | − |
| (25 μM)² | | E. coli and 150 μM for B. subtilis)² | | |
| MM + DA-DA | +++++ | MM + DCS + DA-DA | ++ | ++++ |
| (200 μM)² | | (50 μM for E. coli and | | |
| | | 50 μM for B. subtilis)² | | |
| MM + DA-EDA | +++++ | MM + DCS + DA-EDA | +++ | +++ |
| (100 μM)² | | (50 μM for E. coli and | | |
| | | 800 μM for B. subtilis)² | | |
| MM + DA-ADA | ++++ | MM + DCS + DA-ADA | +++ | ++++ |
| (50 μM)² | | (50 μM for E. coli and | | |
| | | 400 μM for B. subtilis)² | | |
| MM + ELA-LA | − | | | |
| (0.8 mM)² | | | | |
| MM + EDA-DA | − | | | |
| (0.8 mM)² | | | | |
| MM + ADA-DA | − | | | |
| (0.8 mM)² | | | | |
| MM + DA-DA (25 μM)³ | + | | | |
| MM + DA-DA (25 μM) + | + | | | |
| ELA-LA (0.80 mM)³ | | | | |
| MM + DA-DA (25 μM) + | + | | | |
| EDA-DA (0.80 mM)³ | | | | |
| MM + DA-DA (25 μM) + | ++++ | | | |
| EDA-DA (500 μM)³ | | | | |
| MM + DA-DA (25 μM) + | +++ | | | |
| ADA-DA (0.80 mM)³ | | | | |

¹Minimal media for E. coli and B. subtilis was M9 medium + 0.2% glucose and SSM + 0.2% glucose, respectively.
²Initial inoculum was OD₆₀₀ = 0.025
³Initial inoculum was OD₆₀₀ = 0.0025

Growth rescue of a D-alanine-D-alanine ligase double mutant (ΔddlA ΔddlB) of E. coli was tested with varying concentrations of natural and modified dipeptides. Similarly, E. coli and B. subtilis cells were rendered auxotrophic for DA-DA by inhibiting their respective D-alanine-D-alanine ligases with D-cycloserine (DC S). Data represent the average of at least two biological replicates conducted with technical duplicates. +++++, highest culture density achieved within a set; −, no bacterial growth; intermediate values, relative fractions of the highest culture density within a set.

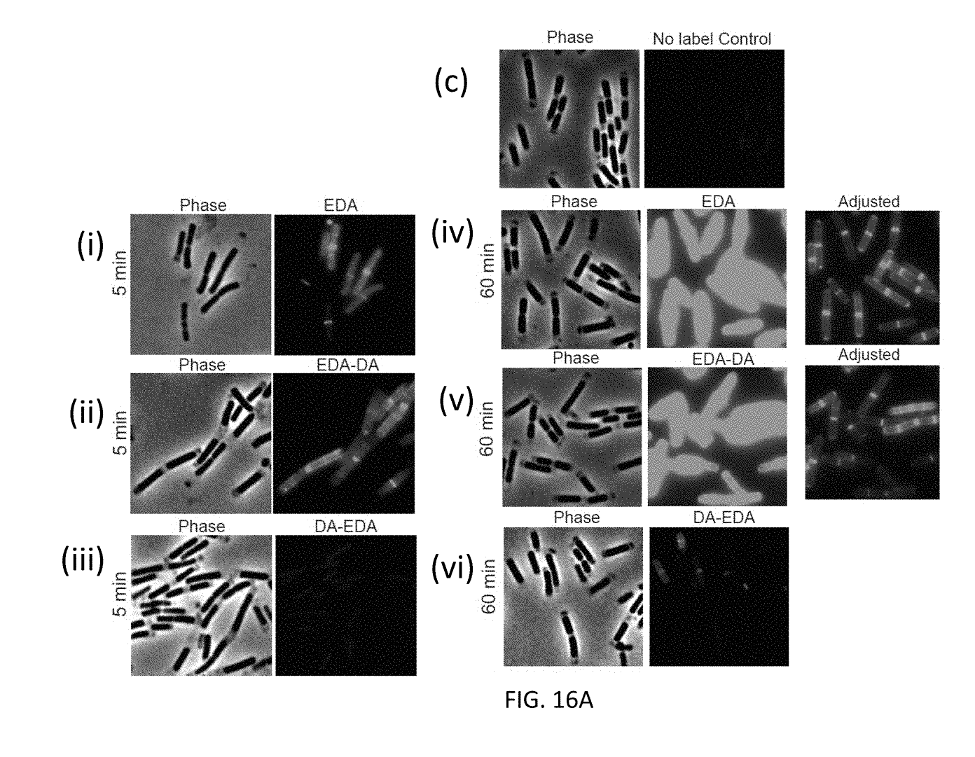

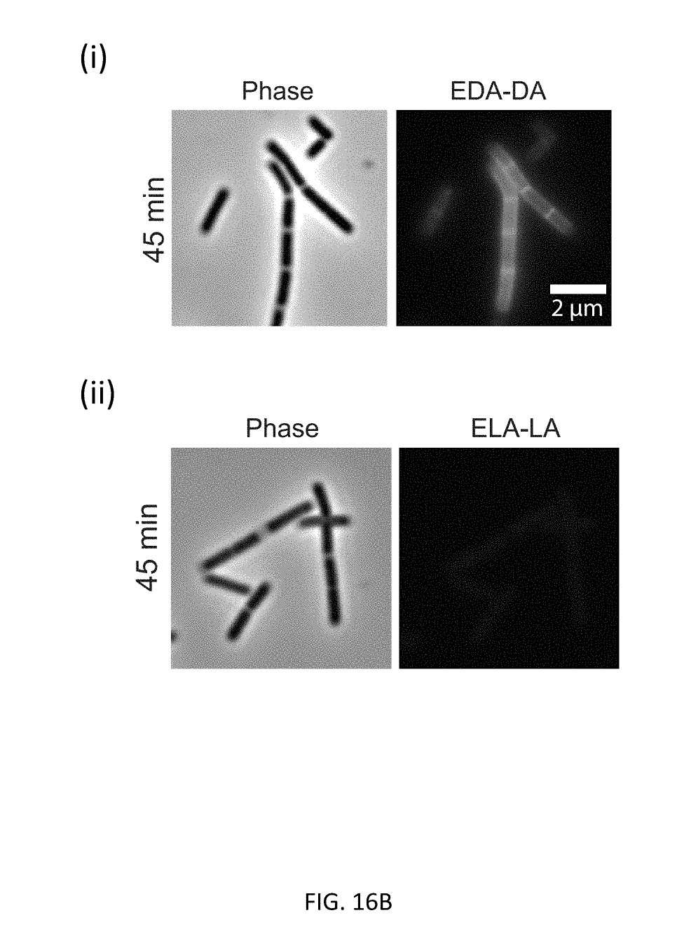

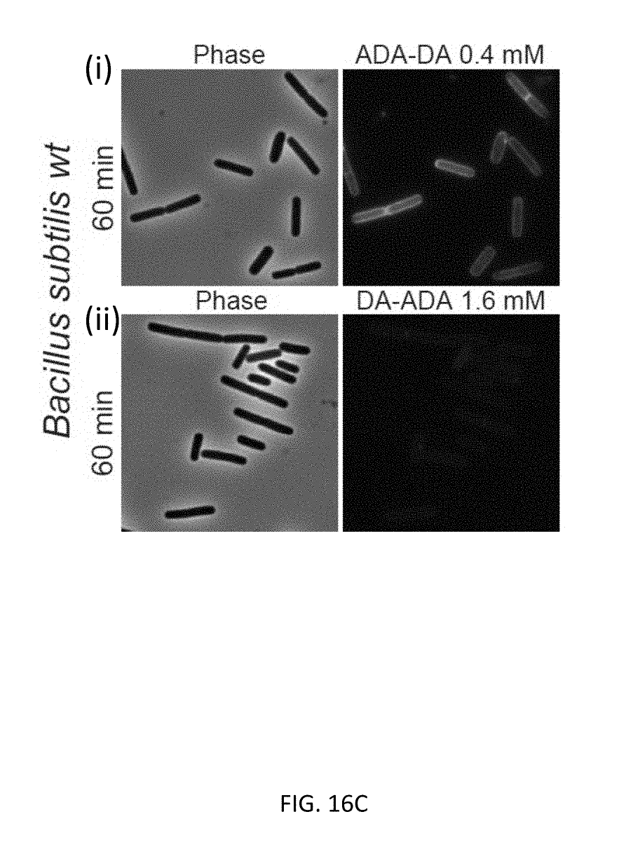

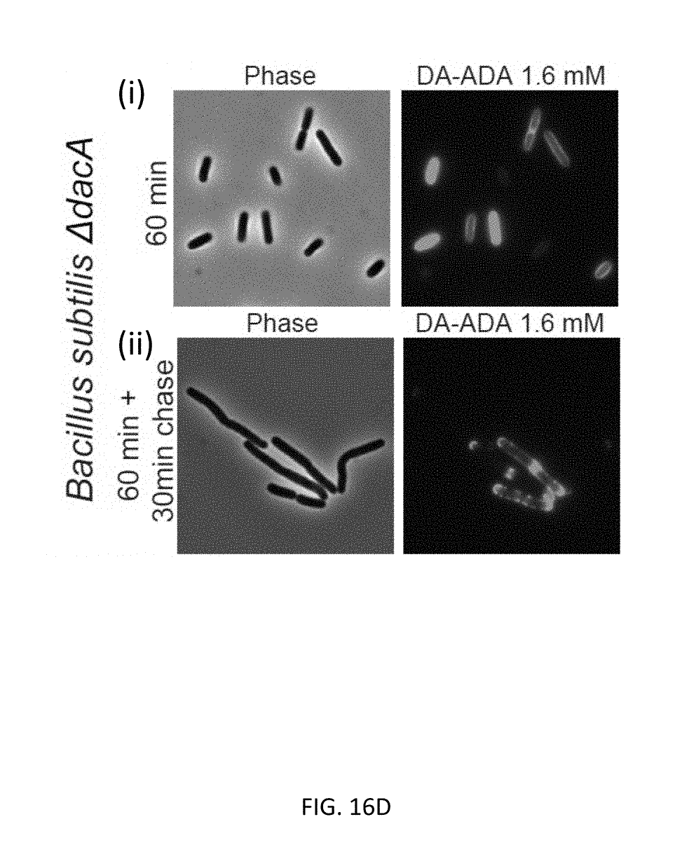

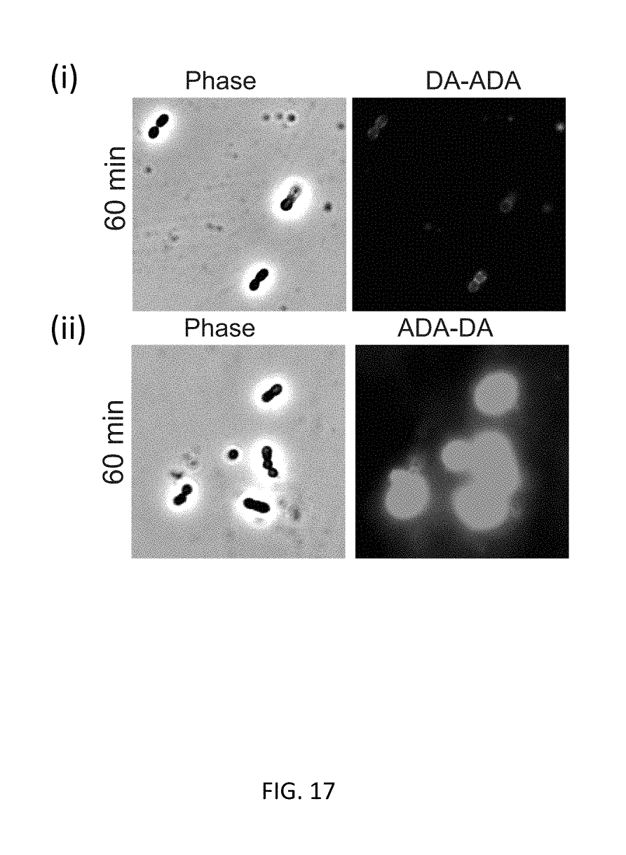

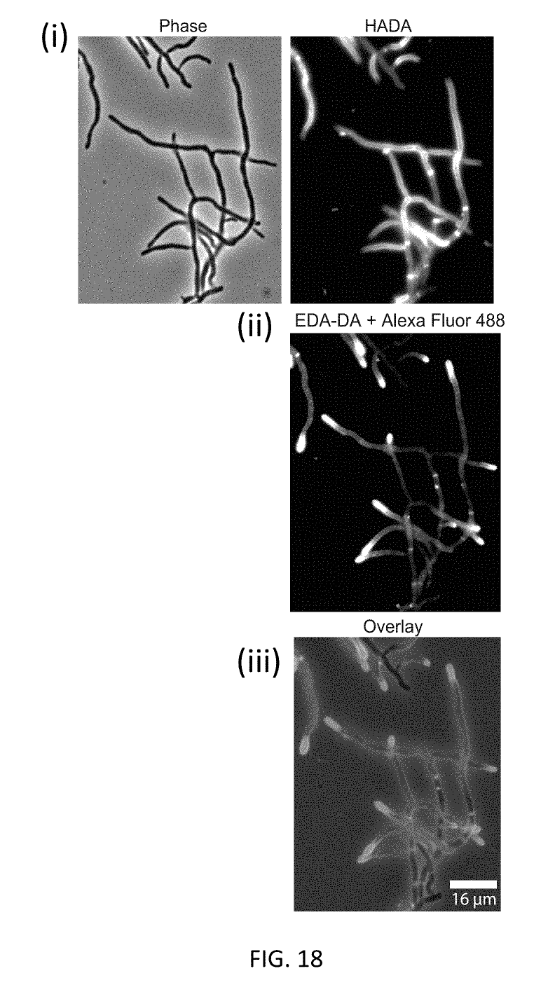

E. coli, B. subtilis, S. pneumoniae and S. venezuelae cells treated by these probes and their conjugate fluorophores, showed time dependent PG labeling patterns that were remarkably similar to single D-amino acid labeling (FIGS. 15-18). Additionally, the non-toxic, copper-less click-chemistry that could be used to detect azide containing probes (ADA-DA or DA-ADA) enabled live cell tracking of PG maturation in B. subtilis and S. pneumoniae (FIGS. 17 and 18). Significantly, the position of the bioorthogonal tag effected the labeling in each species: the tags on the N-terminus of the dipeptide, (EDA-DA or ADA-DA) gave overwhelmingly more signal than the C-terminal tags (DA-EDA or DA-ADA) (see, for examples, FIGS. 15A, 16A, C, and 17). Combined with the rescue data (Table 2), this result indicates that while the N-terminal tag accumulates on maturing PG, the C-terminal tags mark the PG much more transiently, consistent with the rapid C-terminal D-Ala turnover during PG maturation. This is further supported by the recovery of the signal when the major D,D-carboxypeptidase in B. subtilis (DacA) was knocked-out (FIG. 16D). Unlike the single D-amino acid probes, this general method gives the user the versatility of tagging the nascent and older PG differentially through the earlier, cytoplasmic steps of the PG biosynthesis (discussed in the Examples).

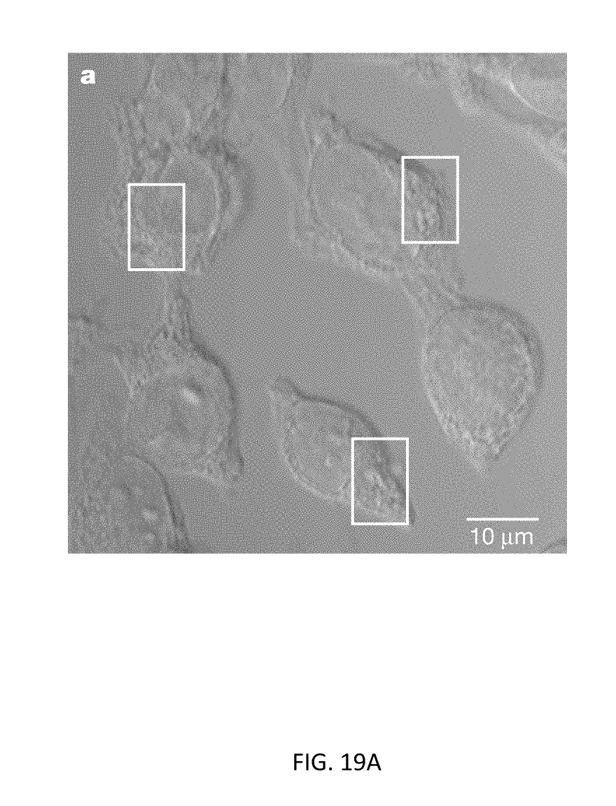

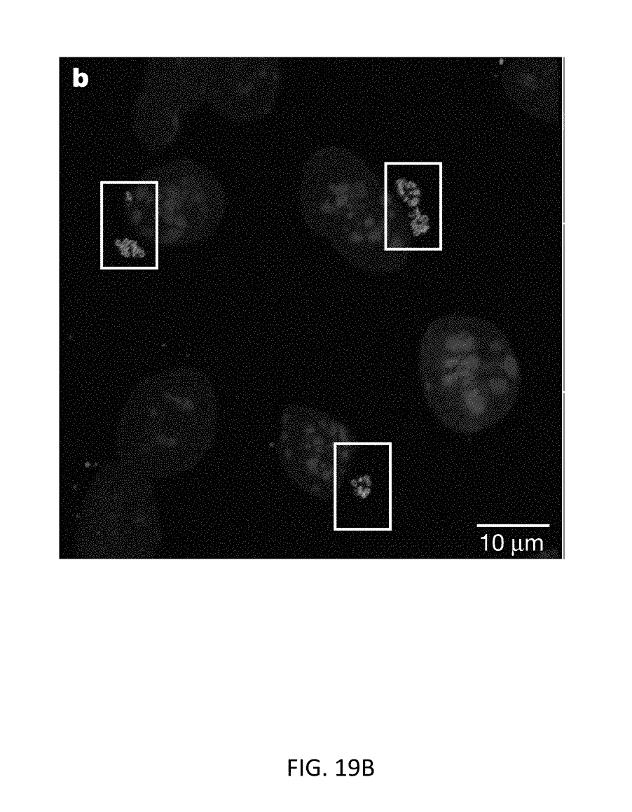

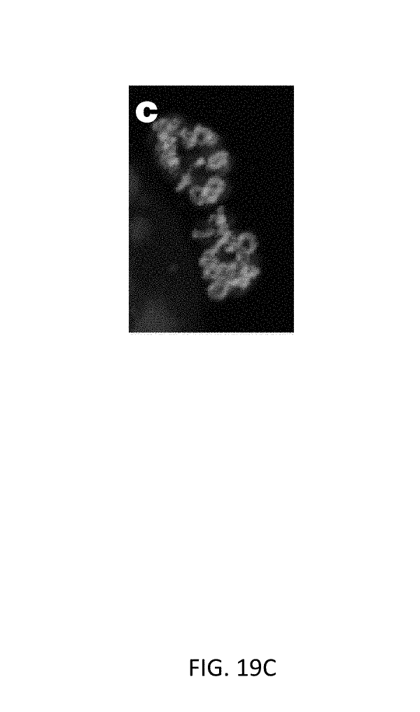

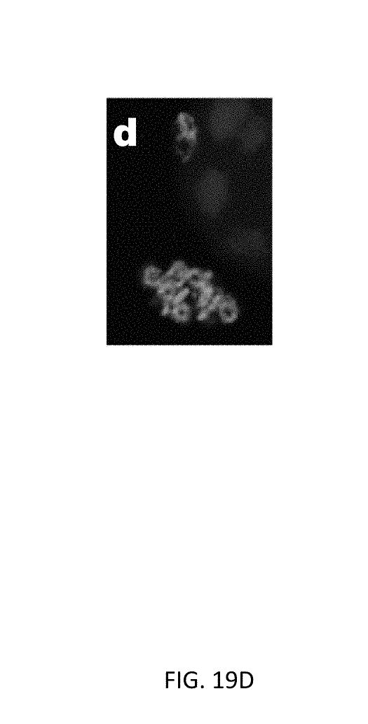

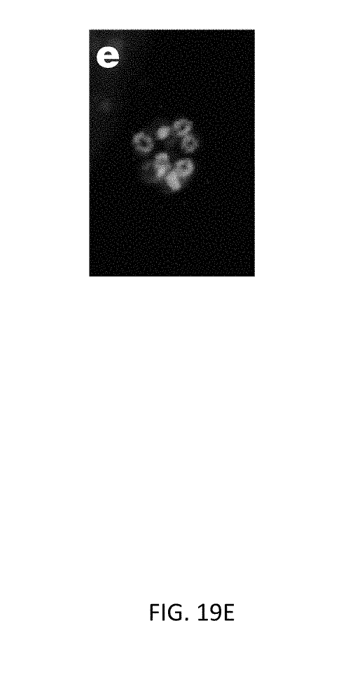

Dipeptide probe uptake and incorporation in intracellular Chlamydia was also studied. L2 mouse fibroblast cells were infected for 18 h with C. trachomatis serovar L2 strain 434/Bu in the presence of 1 mM EDA-DA. Cells were then fixed and permeabilized, and click chemistry was used to attach an Alexa fluorophore modified with a terminal azide group to the alkyne group present on the EDA-DA probe. The probe localized within chlamydial inclusions with individual bacteria clearly discernible (FIG. 19B-E). When co-labeled with antibody to the chlamydial major outer membrane protein (MOW), EDA-DA labeling appeared as either a ring or a single line bisecting MOW-labeled RBs (data not shown). The labeling was arranged in a distinct, ring-like shape, consistent with a cellular division plane and the labeling bore a striking resemblance to images previously obtained for intracellular C. trachomatis stained with antibody generated with Ribi adjuvant. Labeling was only present in Chlamydia-infected cells and only in the presence of probe (data not shown). This result indicates that the majority of labeled chlamydial PG is localized to the septum of dividing RBs. However, we cannot rule out the possibility that low levels of PG exist elsewhere on the bacterium and are simply below the detection limit of fluorescence microscopy. Similar to our results with B. subtilis, we found that incubation with lysozyme for two hours was sufficient to remove EDA-DA labeling within chlamydial inclusions (data not shown), supporting our conclusion that the dipeptide probes are incorporating into chlamydial PG.

To further confirm that the modified probes were being taken up and incorporated into chlamydial PG, we performed plaque assays that allow quantification of intracellular bacterial growth and infectivity. D-cycloserine (DCS) is an inhibitor of cell-wall biosynthesis that targets bacterial alanine racemase and D-alanine-D-alanine ligase and previous studies have shown that Chlamydia growth is inhibited by DCS at millimolar concentrations. Growth inhibition is overcome by supplementation with exogenous D-alanine or DA-DA dipeptide, most likely owing to the exogenous single D-amino acids outcompeting DCS for the binding sites of the chlamydial ligase or, in the case of DA-DA, bypassing the need for the ligase altogether. Various D-amino acids, dipeptides and their corresponding alkyne-modified probes were tested to determine the level of DCS rescue they conferred upon growing C. trachomatis. We found that DA-DA dipeptide and the corresponding modified dipeptides (EDA-DA and DA-EDA) were both capable of rescuing chlamydial plaquing (Table 3), indicating their successful uptake and incorporation by Chlamydia. However, whereas unmodified D-alanine was capable of overcoming the growth inhibitory effects of DCS, the corresponding chemically modified, single D-alanine probe (EDA) was not. These results were consistent with our inability to detect fluorescent labelling of C. trachomatis through single EDA probes (data not shown).

TABLE 3
DCS Chlamydia trachomatis plaque assay in the presence of
natural and modified D-amino acids

| | Amino acid, | |
|---|---|---|
| DCS (μM) | amino acid to DCS ratio | Plaque formation |
| 0 | — | ++++ |
| 294 | No amino acid | − |
| | Dipeptides | |
| | DA-DA, 1:1 | ++ |
| | DA-DA, 10:1 | +++ |
| | DA-EDA, 1:1 | − |
| | DA-EDA, 10:1 | +++ |
| | EDA-DA, 1:1 | − |
| | EDA-DA, 10:1 | ++ |
| | Single amino acids | |
| | D-Ala, 1:1 | ++++ |
| | D-Ala, 1:10 | +++ |
| | EDA, 1:1 | − |
| | EDA, 10:1 | − |

C. trachomatis serovar L2 strain 434/Bu was grown in the plaque assay as previously described in the presence of D-cycloserine (DCS) and varying molar equivalent concentrations of D-alanine (D-Ala), D-alanine-D-alanine (DA-DA), EDA, DA-EDA, and EDA-DA. ++++, complete infection, bacterial growth and lysis of the monolayer; +++, numerous large plaques but less than complete lysis of the monolayer; ++, numerous small plaques; +, few (˜10-20) small plaques; −, no plaque formation (no bacterial growth). Data represent the average of three biological replicates and each experiment was conducted with technical duplicates.

Despite rescue of chlamydial growth by both EDA-DA and DA-EDA in the DCS plaque assay, we initially were not able to label chlamydial PG with DA-EDA. Similarly, we were unable to obtain labeling with DA-EDA in E. coli. We reasoned that the inability to label Chlamydia with DA-EDA was due to the removal of the terminal, modified EDA amino acid from the PG pentapeptide stem during either transpeptidation or carboxypeptidation (FIG. 14B).

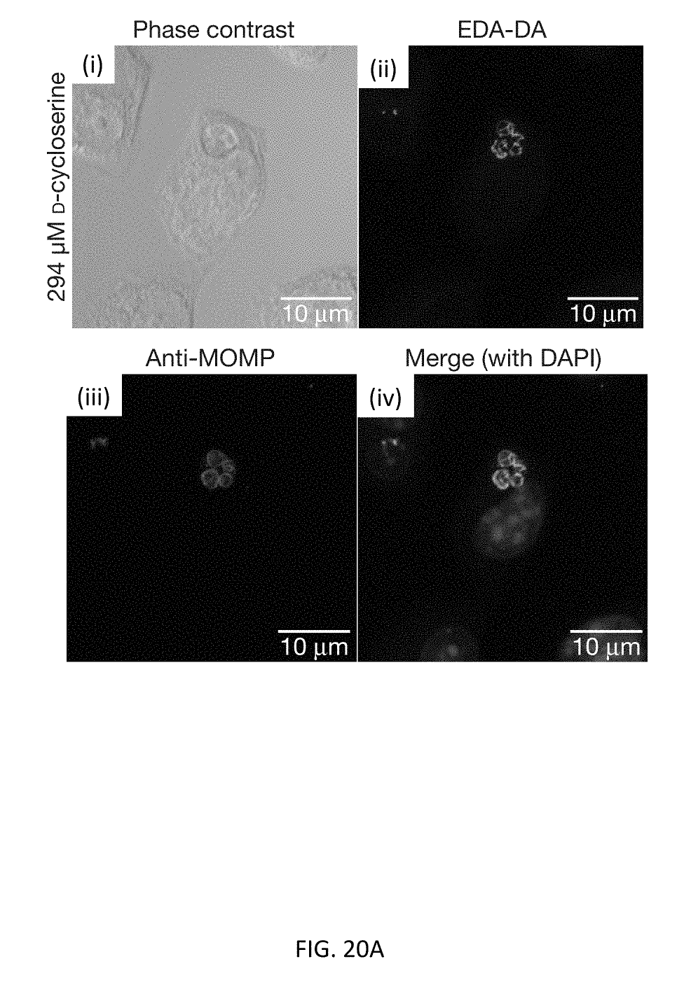

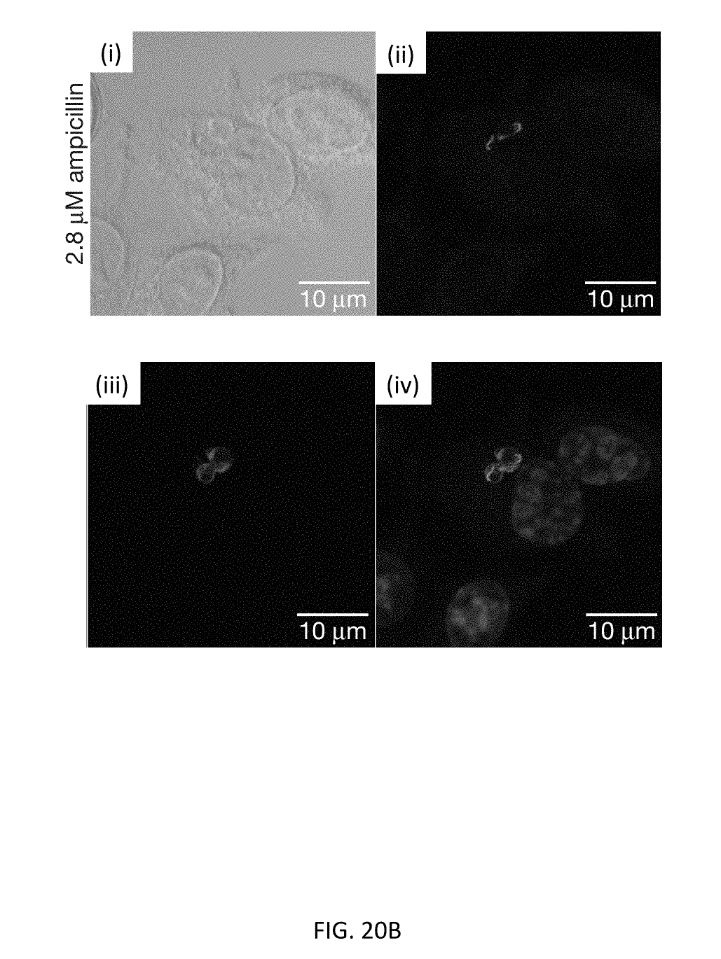

To test this hypothesis and to further validate that our probes were incorporated into C. trachomatis PG, we conducted EDA-DA and DA-EDA labeling studies in the presence of two antibiotics that block PG biosynthesis: DCS, a competitive inhibitor of both alanine racemase and D-alanine-D-alanine ligase, and ampicillin, an inhibitor of PG transpeptidases/carboxypeptidases. When grown for 18 h in the presence of either antibiotic, inclusions contained enlarged, aberrant RBs. The presence of fewer bacteria per inclusion is indicative of a pre-division block, due to the absence of transpeptidation, and is consistent with the literature. In the presence of DCS and 1 mM EDA-DA, fluorescent PG was discernible within aberrant RBs (FIG. 20A). This result indicates that EDA-DA was capable of partly substituting for DA-DA after depletion of the bacterium's natural dipeptide pool and confirms the DCS plaquing assay results. EDA-DA labeling intensity seemed unaffected by inhibition of PG transpeptidation/carboxypeptidation with ampicillin (data not shown), indicating that probe incorporation is not dependent on transpeptidation and does not occur in the periplasm in Chlamydia. When imaged by epifluorescence, labeling of aberrant bodies grown in the presence of ampicillin often appeared punctate, owing to the enlarged PG ring structures that no longer exist within a single focal plane (data not shown). z-stacks taken of the ampicillin-treated aberrant RBs clearly revealed labelled PG sequestered to an equatorial region where the bacterial division plane would normally form (FIG. 20B). Fluorescence labeling of Chlamydia with DA-EDA was only observed when transpeptidation/carboxypeptidation was inhibited with ampicillin (data not shown). DA-ADA labeling of a B. subtilis D,D-carboxypeptidase mutant (ΔdacA) confirmed this finding; labeling was greatly increased compared to the parental, wild-type strain and was not significantly turned over as the labelled cells were allowed to grow (FIGS. 16C, D). These observations indicate that PG modifications (through transpeptidation and/or carboxypeptidation) occur in vivo in Chlamydia, as inhibition of these modifications would preserve the terminal D-alanine in the stem peptide, thus allowing for labeling of PG with DA-EDA.

In view of the foregoing, disclosed herein are compositions for and methods of covalently labeling PG in live bacterial cells. This method works very efficiently in both Gram-positive and Gram-negative organisms (Gram-negative organisms represent a liability for approaches using fluorescently modified vancomycin/ramoplanin), and the probe substrates do not appear to be toxic to cells and show no adverse effects on cell morphology, even at concentrations as high as 1 mM. The probes rapidly label sites of active peptidoglycan biosynthesis and can be used in time-lapse and dual labeling experiments.

Thus, compositions and methods have been described herein for in situ labeling/probing of PG synthesis in bacteria with fluorescent D-amino acids (FDAAs), as well as for screening for bacterial cell wall-acting and/or cell wall-disrupting agents. The FDAAs are based upon D-amino acids (DAAs) derivatized to covalently include a small fluorophore. As such, the FDAAs can be directly incorporated into bacterial cell walls during PG biosynthesis, as occurs at sites of cell division in actively dividing cells.

The disclosed compositions include a modified dipeptide having D-amino acids covalently attached to a bioorthogonal tag. In some embodiments, the bioorthogonal tag is ADA, AHA, EDA or EDTA. In some embodiments, D-amino acids of the modified dipeptide are selected from the group consisting of 3-amino-D-Ala, D-Ala, D-Asp, D-Cys, D-Glu and D-Lys, or a combination thereof.

The disclosed compositions also include a muramylpentapeptide precursor unit comprising an N-acetyl muramic acid (NAM) moiety having a stem peptide of three to five amino acids. The one or more of the amino acids in the stem peptide includes a modified dipeptide of any one of the preceding claims and optionally an additional modified dipeptide, wherein the additional modified dipeptide includes a clickable D-amino acid.

The disclosed compositions also include a peptidoglycan unit comprising the muramylpentapeptide precursor unit of claim 4 covalently linked to an N-acetyl glucosamine (NAG) moiety.

In another aspect of the invention, a live bacterial organism comprising a bacterium having a modified cell wall comprising having peptidoglycan containing at least one modified dipeptide as described above and optionally at least one additional modified dipeptide is provided. The at least one additional dipeptide includes a clickable D-amino acid.

In another aspect of the invention, a method of assessing bacterial cell wall synthesis in real time is provided. The method includes the step of providing live bacteria with a first amount of at least one modified dipeptide as described above, and optionally a second amount of at least one additional modified dipeptide that includes a clickable D-amino acid, under conditions sufficient for bacterial cell wall synthesis. The bacteria covalently incorporate the at least one modified dipeptide and optionally the at least one additional modified dipeptide into a stem peptide of peptidoglycan of the bacterial cell wall. In some aspects, first amount and second amount comprise a first concentration and a second concentration, respectively, wherein the first and second concentrations range from about and including 0.10 mM to about and including 1 mM. In other aspects, the method further includes the step of detecting the at least one modified dipeptide, and optionally the at least one additional modified dipeptide incorporated into the stem peptide. In these methods, the bacteria can be Gram-positive bacteria or Gram-negative bacteria.

In another aspect of the invention, a method of screening for a putative cell wall-acting agent is provided. The method includes the step of co-contacting bacteria with an effective amount of an agent and an amount of at least one modified dipeptide as described above, and optionally an amount at least one additional modified dipeptide includes a clickable D-amino acid, under conditions sufficient to permit ongoing peptidoglycan biosynthesis in a bacterial cell wall. The agent includes a cell wall-acting agent if the agent interferes with ongoing peptidoglycan biosynthesis in the bacterial cell wall. The method further includes the step of detecting one or more modified dipeptides incorporated in the bacterial cell wall. In one respect of this extended method, the step of detecting one or more modified dipeptides incorporated in the bacterial cell wall includes the step of post-labeling the bioorthogonal tag with a label and the step of visualizing the one or more labeled dipeptides with microscopy. In another embodiment of this method, an additional step is provided. The additional step includes comparing the amount and/or identity of incorporated modified dipeptides in the bacterial cell wall resulting from contacting the bacteria with the agent with the corresponding amount and/or identity of incorporated modified dipeptides in a bacterial cell wall resulting from contacting the bacteria with a known cell wall-acting agent.

In another aspect of the invention, a method of screening for a putative cell wall-disrupting agent is provided. The method includes the step of contacting modified bacteria with an amount of an agent. The agent comprises a cell wall-disrupting agent if the agent weakens integrity of peptidoglycan in an existing bacterial cell wall. The modified bacteria have a modified cell wall containing modified peptidoglycan having at least one stem peptide containing at least one modified dipeptide as described above, and optionally at least one additional modified dipeptide that includes a clickable D-amino acid. In some embodiments, the method includes the additional step of detecting one or more modified dipeptides disrupted in the bacterial cell wall. In some embodiments, the step of detecting one or more modified dipeptides disrupted in the bacterial cell wall includes the step of post-labeling the bioorthogonal tag with a label and the step of visualizing the one or more labeled dipeptides with microscopy. In another embodiment of this method, an additional step is provided. The additional step includes comparing the amount and/or identity of disrupted D-amino acids in the bacterial cell wall resulting from contacting the bacteria with the agent with the corresponding amount and/or identity of disrupted D-amino acids in a bacterial cell wall resulting from contacting the bacteria with a known cell wall-disrupting agent.

In the foregoing methods, the bacteria can be Gram-positive bacteria or Gram-negative bacteria.

In another aspect of the invention, a method of identifying bacteria is provided. The method includes three steps. The first step includes contacting live bacteria with an amount of at least one modified dipeptide as described above, and optionally an amount of at least one additional modified dipeptide that includes a clickable D-amino acid, under conditions sufficient for ongoing bacterial cell wall synthesis, wherein the bacteria covalently incorporate into peptidoglycan of a bacterial cell wall the at least one modified dipeptide, and optionally the at least one additional modified dipeptide, wherein each of the least one modified dipeptide and optionally the at least one additional modified dipeptide comprises a distinct bioorthogonal tag. The second step includes post-labeling each distinct bioorthogonal tag with spectrally distinct label. The third step includes visualizing the spectrally distinct labels to determine an incorporation pattern of the at least one modified dipeptide, and optionally the at least one additional modified dipeptide, wherein the incorporation pattern identifies the bacteria.

In another aspect of the invention, a kit for incorporating modified dipeptides into live bacteria is provided. The kit includes two components. The first component includes at least one modified dipeptide as described above. The second component includes a positive bacterial control and optionally a negative bacterial control. The positive bacterial control has at least one modified dipeptide as described above incorporated into a stem peptide of peptidoglycan of the bacterial cell wall. The optional negative bacterial control, if included, does not have the modified dipeptide as described above incorporated into a stem peptide of peptidoglycan of the bacterial cell wall. In some embodiments, the kit further includes at least one clickable D-amino acid. In some embodiments, the kit further includes at least one reagent for post-labeling the bioorthogonal tag.

### EXAMPLES

The invention will be more fully understood upon consideration of the following non-limiting examples, which are offered for purposes of illustration, not limitation.

### Example 1. Evaluation of Incorporation of Fluorescently-Labeled D-Amino Acids and L-Amino Acids in Bacteria

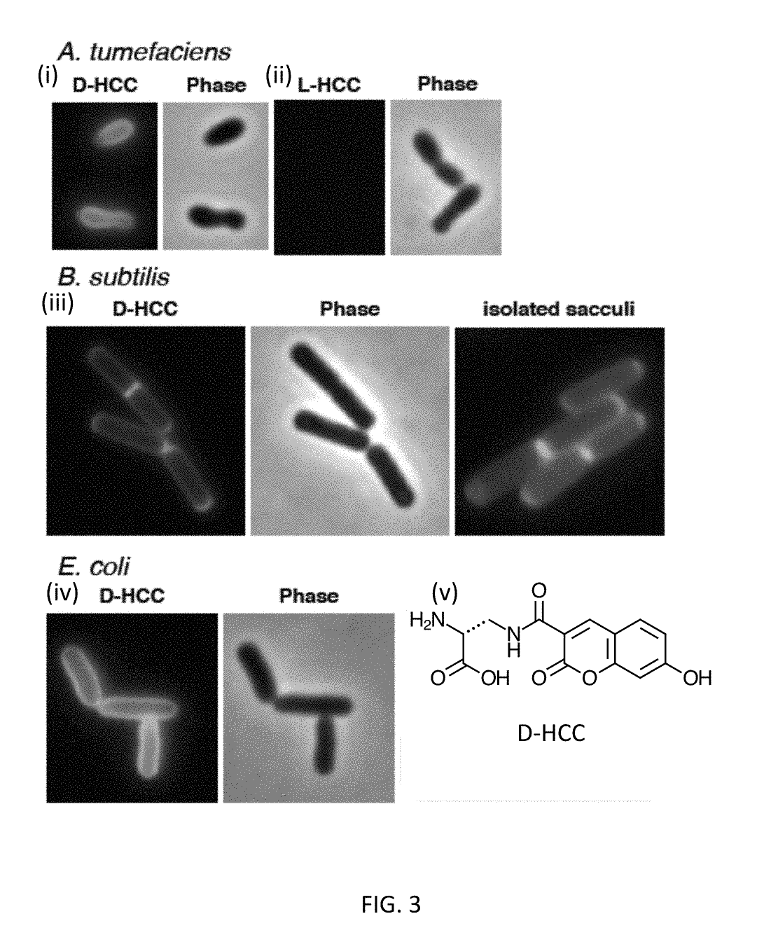

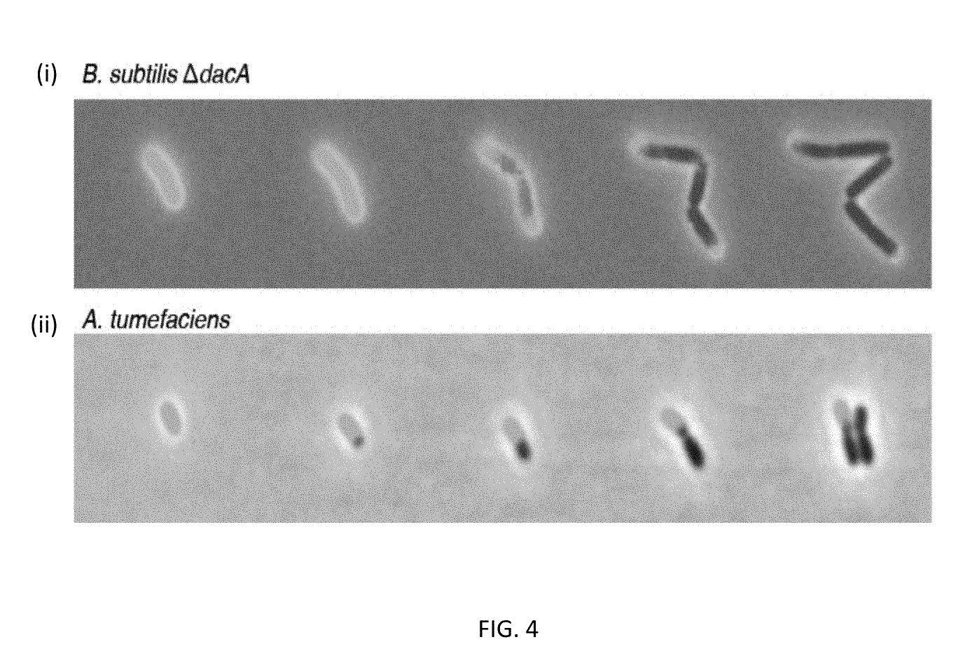

Control experiments were carried out in A. tumefaciens, B. subtilis and E. coli with fluorescent D-Ala (D-HCC) and fluorescent L-Ala (L-HCC). For example, experiments in A. tumefaciens revealed that only D-HCC was incorporated into the cell wall. This observation was true for all strains tested. Likewise, experiments with B. subtilis revealed predominant labeling at the septum, a result consistent with this being the site of active cell wall synthesis. Subsequent isolation of peptidoglycan from these cells also revealed that isolated sacculi retained the fluorescent label (FIG. 3).

In addition, experiments with a B. subtilis dacA mutant (DacA is a D,D-carboxypeptidase that cleaves the terminal D-Ala from the peptide stem) resulted in uniform labeling of the cell wall, which suggested the dominant mode of labeling in B. subtilis is at the terminal position of the peptide stem.

A series of pulse-chase experiments were performed in which exponentially growing cells were diluted and treated with one of the D-Ala probes (250 μM-500 μM), The cells were incubated until saturation, washed, and placed on LB-containing agar pads and imaged at 5-minute intervals for 12-18 hours (FIG. 4).

An experiment in B. subtilis revealed that fluorescence persisted at the cell poles, an observation that is consistent with the notion that there is not active cell wall synthesis taking place in these regions (FIG. 4). Interestingly, the labeling pattern observed with A. tumefaciens provides supporting evidence for a mode of growth that involves budding as no signal dilution from the mother cell is observed as recently shown.

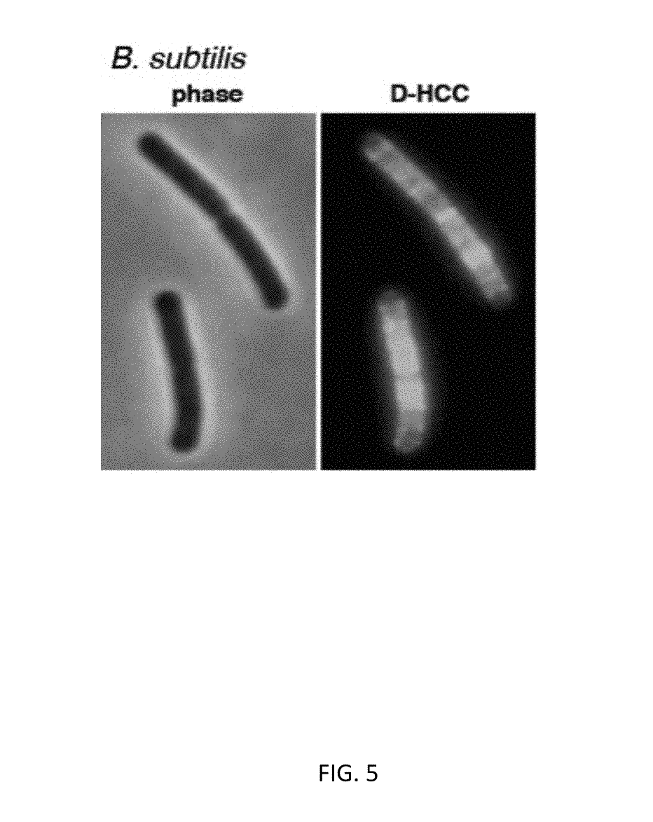

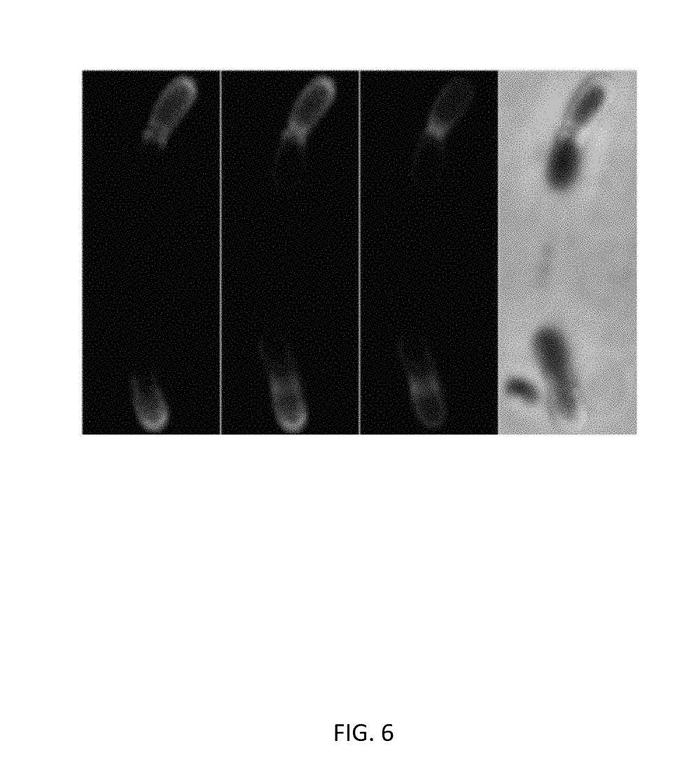

More significantly, short exposures to FDAA derivatives have proven to be optimal for imaging the sites of active cell wall biosynthesis. For example, when a culture of exponentially growing cells is contacted with either D-NBD or D-HCC, and the cells were pulsed for 2%-8% of their usual generation time and immediately fixed, sites of active synthesis were clearly visible in evolutionarily distinct bacteria such as E. coli, B. subtilis, A. tumefaciens, L. lactis, M. conglomeratus, C. crescentus and S. aureus. Significantly, with experiments conducted in B. subtilis, these short pulses result in a staining pattern that appears to be consistent with the helical pattern that has been observed with fluorescently modified vancomycin/ramoplanin (FIG. 5).

Finally, experiments were performed with the labeled D-Ala derivative in a dual-labeling format. For example, A. tumefaciens cells were incubated for a period of 4 minutes in 500 μM D-NBD, followed by washing and incubation for 4 minutes in 500 μM D-HCC. The excess dye was removed and the cells were pelleted and fixed. The fluorescence micrographs reveal distinct patterns of growth, in terms of polar growth and septal synthesis, based on the age of the daughter cell (FIG. 6).

### Example 2. Fluorescent D-Amino Amino Acids: Synthesis and Biological Properties

Methods

Synthesis of Fluorescent D-Amino Amino Acids (FDAAs)

HADA/HALA: To a flame-dried flask, 7-hydroxycoumarin-3-carboxylic acid (HCC) was added in anhydrous DMF (14.5 mL, 0.1 M) under an atmosphere of argon. Carbonyldiimidazole (236 mg, 1.455 mmol) was added in one portion and stirred at room temperature (RT) for 2 hours.

Boc-D-2,3-diaminopropionic acid (for HADA) or Boc-L-2,3-diaminopropionic acid (for HALA) (297 mg, 1.455 mmol) was added in one portion and the reaction mixture was allowed to stir at RT overnight (17 hours). The majority of the solvent was removed in vacuo, and the product was diluted with EtOAc (100 ml) and washed with 1N HCl (50 ml) and water (100 ml). The water layers were combined and back-extracted with EtOAc (50 ml) to prevent loss of product due to an emulsion. The organic layers were combined, washed with brine (50 ml), dried over Na₂SO₄, filtered, and the solvent was removed in vacuo. Without further purification the crude product was treated with trifluoroacetic acid/dichloromethane (50:50, 10 ml) for 30 minutes at RT, and the solvent was removed in vacuo. The product was purified via reverse-phase HPLC with 10%-90% MeCN/H₂O. The pure fractions were concentrated in vacuo, and the product was redissolved in 1N HCl/MeCN and lyophilized to yield the desired product as a pale yellow solid (297 mg, 62% for HADA and 277 mg, 58% for HALA). [α]²⁰_{D}−21.8° (c 2.2, DMSO-d6); HRMS-ESI-TOF m/z calc'd for C₁₃H₁₂O₆N₂ ([M+H]⁺): 293.0774, Found 293.0774; HPLC: tR=5.96 min (10-90% MeCN/H₂O over 10 minutes); ¹H NMR (400 MHz, DMSO-d6) δ=2.46 (s, 1H), 3.69-3.77 (m, 1H), 3.79-3.87 (m, 1H), 4.07 (t, J=5.6 Hz, 1H), 6.85 (d, J=2.0 Hz, 1H), 6.89 (dd, J=2.0, 8.4 Hz, 1H), 7.89 (d, J=8.8 Hz, 1H), 8.43 (br s, 3H), 8.76 (s, 1H), 8.86 (t, J=6.2 Hz, 1H), 11.34 (br s, 1H); ¹³C NMR (100 MHz, DMSO-d6): δ=52.1, 102.3, 111.3, 113.5, 115.0, 132.5, 148.8, 156.8, 161.2, 163.1, 164.6, 169.7; the signal for one carbon was overlapping with the solvent peak.

NADA/NALA: Boc-D-2,3-diaminopropionic acid (for NADA) or Boc-L-2,3-diaminopropionic acid (for NALA) (100 mg, 0.49 mmol) and sodium bicarbonate (123 mg, 1.47 mmol) were dissolved in water (1.8 ml) and heated to 55° C. in water bath. A solution of 4-chloro-7-nitrobenzofurazan (NBD, 108 mg, 0.539 mmol) in methanol (8.5 ml) was added dropwise over 10 minutes. Care was taken at all times to avoid excessive exposure to light during the reaction and workup. The reaction was allowed to stir at 55° C. for 1 hour. The solvent was removed in vacuo and acidified with 1N HCl. The aqueous mixture was extracted with dichloromethane (50 ml per extraction×3 extractions) and the organic extracts were washed with brine (50 ml), dried over Na₂SO₄, filtered, and the solvent was removed in vacuo. Without further purification the crude product was treated with 4N HCl/dioxane (10 ml) for 1 hour at RT, and the solvent was removed in vacuo. The product was purified via reverse-phase HPLC with 20%-90% MeCN/H₂O. The pure fractions were concentrated in vacuo, and the product was redissolved in 1N HCl/MeCN and lyophilized to yield the desired product as a bright orange solid (105 mg, 71% for both NADA and NALA). [α]²⁰_{D}=−32° (c 1.1, DMSO-d6) HRMS-ESI-TOF m/z calc'd for C₉H₉O₅N₅ ([M+H]⁺): 268.0682, Found 268.0680; HPLC: tR=5.02 minutes (20-90% MeCN/H₂O over 10 minutes). ¹H NMR (400 MHz, DMSO-d6): δ=4.06 (m, 2H), 4.29 (m, 1H), 6.61 (d, J=8.0 Hz, 1H), 8.56 (d, J=8.0 Hz, 1H), 8.66 (br s, 3H), 9.32 (br s, 1H); ¹³C NMR (100 MHz, DMSO-d6): δ=43.4, 51.5, 100.5, 122.5, 138.1, 144.4, 144.9, 145.2, 169.2.

FDL: To a flame dried flask was added Nα-Boc-D-Lys-OH (19.3 mg, 0.078 mmol) and fluorescein isothiocyanate (25 mg, 0.065 mmol) in dry DMF (0.65 ml). The reaction was stirred under argon at room temperature for 4 hours. The solvent was removed in vacuo. The residue was redissolved in ethyl acetate (10 ml), washed with 1N HCl (10 ml) and brine (10 ml), and dried over anhydrous sodium sulfate. The solvent was again removed in vacuo, and the crude product was treated with trifluoroacetic acid/dichloromethane (1:1) for 0.5 hours. The acid was removed in vacuo, and the product was purified by reverse phase HPLC with 30%-45% MeCN/H₂O. The pure fractions were lyophilized to yield the product as a dark yellow solid (25.0 mg, 72%). [α]²⁰_{D}=−7.1° (c 0.72, MeOH-d4); HMRS-ESI-TOF m/z calc'd for C₂₇H₂₆N₃O₇S ([M+H]+): 536.1492, Found 536.1470; HPLC: tR=5.08 min (30%-45% MeCN/H₂O over 10 minutes); ¹H NMR (400 MHz, MeOD-d4): δ=1.50-1.63 (m, 2H), 1.78 (quintet, J=7.0 Hz, 2H), 1.90-1.99 (m, 1H), 2.00-2.10 (m, 1H), 3.66 (br s, 2H), 4.00 (t, J=6.3 Hz, 1H), 6.59 (dd, J=8.8 Hz, 2H), 6.73 (s, 2H), 6.74 (d, J=8.90 Hz, 2H), 7.18 (d, J=8.2 Hz, 1H), 7.76 (d, J=8.2 Hz, 1H), 8.17 (s, 1H); ¹³C NMR (100 MHz, MeOD-d4): δ=23.3, 29.5, 31.3, 45.0, 53.9, 103.5, 111.9, 114.1, 120.5, 126.1, 129.2, 130.5, 131.8, 142.5, 154.6, 162.1, 171.0, 171.9, 182.9.

TDL: To a flame dried flask was added Na-Boc-D-Lys-OH (3.3 mg, 0.0134 mmol), 5-(and 6-) carboxytetramethylrhodamine succinimidyl ester (5 mg, 0.0095 mmol), and diisopropylethylamine (2.5 μl, 0.0143 mmol) in dry DMF (0.2 ml). The reaction was stirred under argon at room temperature overnight. The solvent was removed in vacuo, and the crude mixture was treated with trifluoroacetic acid/dichloromethane (1:1) for 0.5 hours. The reaction was dried in vacuo, and purified by reverse-phase HPLC with 20%-40% MeCN/H₂O. The pure fractions were lyophilized to yield the product as a deep red solid (4.6 mg, 61%). [α]²⁰_{D}=−210° (c 0.20, MeOH-d4); HRMS-ESI-TOF m/z calc'd for C₁₃H₃₅N₄O₆ ([M+H]⁺): 599.2557, Found 599.2559; HPLC: tR=7.86 and 9.06 minutes (2 isomers isolated results from mixed isomer starting material) (20%-40% MeCN/H₂O over 10 minutes); ¹H NMR (400 MHz, MeOD-d4): δ=1.25-1.35 (m, 2H), 1.46-1.62 (m, 2H), 1.68 (quintet, J=7.2 Hz, 2H), 1.75-2.05 (m, 2H), 3.41 (t, J=7 Hz, 1H), 3.92 (t, J=Hz, 1H), 7.01 (d, J=2 Hz, 2H), 7.05 (dd, J=2.0 Hz, 9.4 Hz, 2H), 7.13 (d, J=9.4 Hz, 2H), 7.81 (d, J=1.5 Hz, 1H), 8.19 (dd, J=1.5 Hz, 8.6 Hz, 1H), 8.39 (d, J=8.6 Hz, 1H).

Spectral Characteristics of FDAAs

Excitation and emission spectra of FDAAs (500 μM in 100 mM Tris pH 7.0) were determined in black 96-well polystyrene plates (Corning) using top-read function of a Spectra Max M2 plate reader. The excitation and emission spectra were measured in separate runs within a range of 200 nm and with increments of 1 nm.

Click Chemistry

EDA and ELA, and Sulfo-Cy3-Azide were gifts from Boaopharma and Lumiprobe, respectively. AZA and “clickable” Alexa 488 Fluors were purchased from Iris Biotech GmbH and Invitrogen, respectively. The Cu(I) catalyzed click chemistry was performed using the chemicals supplied by Invitrogen following their standard protocol once the cells had been fixed with EtOH (70% v/v) and permeabilized with methanol (100% v/v).

Growth Conditions

Strain characteristics and growth conditions are described in Table 4.

TABLE 4
Strains, their predicted PG chemotypes and conditions for growth and labeling.

| | | | | PG | | [HADA] | Temperature | |
|---|---|---|---|---|---|---|---|---|
| Species | Strain | Source | Gram | Chemotype^{[5]} | Media | (mM)* | (° C.) | Aeration |
| Bacillus subtilis | PY76 | Daniel Kearns | + | A1γb^{[7]} | LB^{[6]} | N/A | 37 | Y |
| | | IUB | | | | | | |
| Bacillus subtilis | PY76, | Daniel Kearns | + | A1γb | LB | 1 | 37 | Y |
| | dacA::cam | IUB | | | | | | |
| Bacillus subtilis | CU1065 | John Helmann | + | A1γb | LB | N/A | 37 | Y |
| | | CU | | | | | | |
| Bacillus subtilis | HB0048 - | John Helmann | + | A1γb | LB | N/A | 37 | Y |
| | CU1065, | CU | | | | | | |
| | dltA::spc | | | | | | | |
| Brachybacterium | | IUB culture | + | A4γ | LB | 1 | 30 | Y |
| conglomeratum | | collection | | | | | | |
| Lactococcus lactis | | IUB culture | + | A4α | LB | 1 | 37 | Y |
| | | collection | | | | | | |
| Staphylococcus | | IUB culture | + | A3α | LB | 1 | 37 | N |
| aureus | | collection | | | | | | |
| Streptococcus | IU1945 | Malcolm | + | A1α & | BHI^{[9]} | 0.5 | 37 | N |
| pneumoniae | | Winkler IUB | | A3α^{[8]} | | | | |
| Streptomyces | | Justin Nodwell | + | A3γ | LB | 0.5 | 30 | Y |
| venezuelae | | MU | | | | | | |
| Agrobacterium | | Yves Brun | − | A1γ | LB | 1 | 26 | Y |
| tumefaciens | | IUB | | | | | | |
| Burkholderia | | Ann Hirsch | − | A1γ | LB | 0.5 | 30 | Y |
| phytofirmans | | UCLA | | | | | | |
| Caulobacter | YB5630 - | Yves Brun | − | A1γ | PYE^{[10]} | 0.5 | 30 | Y |
| crescentus | CB15 Δrsa | IUB | | | | | | |
| | ΔhfsA | | | | | | | |
| Escherichia coli | MG1655 | Patricia Foster | − | A1γ | LB | 1 | 37 | Y |
| | | IUB | | | | | | |
| Synechocystis sp. | | David Kehoe | − | A1γ | BG-11^{[11]} | 1 | 26 | CO₂ + |
| PCC 6803 | | IUB | | | | | | artificial |
| | | | | | | | | sunlight |
| Verrucomicrobium | | Naomi Ward | − | A1γ | VM^{[12]} | 0.25 | 30 | Y |
| spinosum | | UW | | | | | | |

Key:
IUB = Indiana University Bloomington
CU = Cornell University
MU = McMaster University
UCLA = University of California, Los Angeles
UW = University of Wyoming
*Short pulse concentrations. By default, a normalized 1% (v/v) DMSO concentration was used for all labeling experiments.

For any experiment involving FDAAs and/or CDAAs, DMSO was added to the growth media to a final concentration of 1% to help solubilize the FDAAs and/or CDAAs. Presence of 1% DMSO did not affect labeling or growth in bacteria tested. When necessary, chloramphenicol or spectinomycin was added to the growth media at 5 μg/ml or 100 μg/ml, respectively. Strains were maintained on plates containing growth media with 1.5% agar.

Growth Curves

For growth curves, exponentially growing E. coli, A. tumefaciens and A. tumefaciens and B. subtilis ΔdacA were diluted to OD₆₀₀ₙₘ 0.05 into wells of polystyrene 24-well plates (Falcon) containing 750 μl LB with 1% DMSO or 1% DMSO+FDAAs (250 μM-1 mM). The absorbance at 600 nm was read every 5 minutes for 18 hours in a BIO-TEK Synergy HT Plate Reader (30° C., static).

Short Labeling Pulses and Fluorescence Microscopy

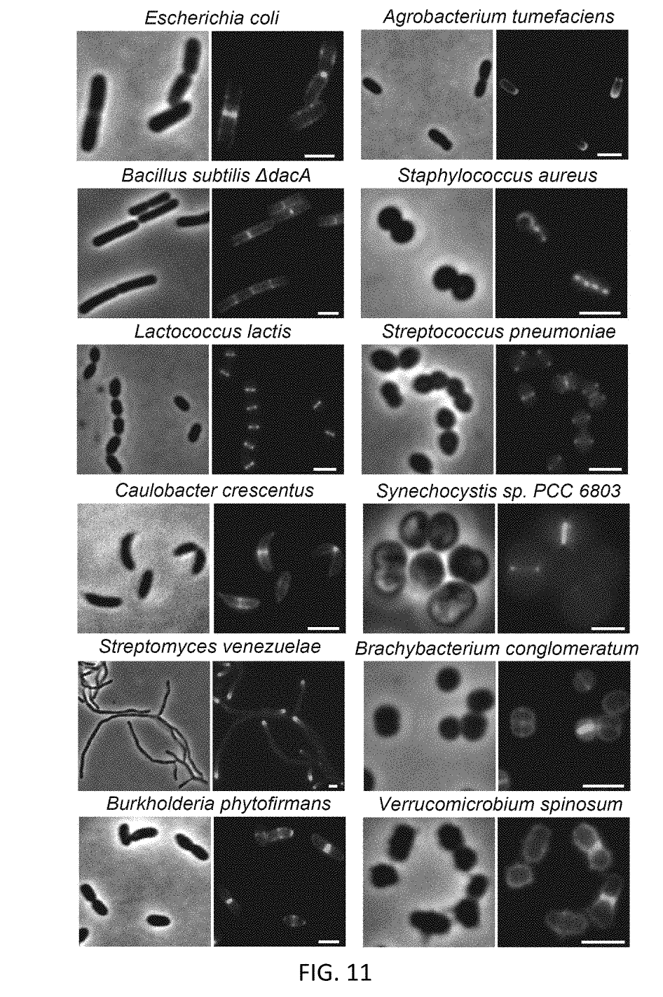

For short labeling pulses, exponentially growing cells were screened for the minimum concentration of FDAAs or CDAAs (250 μM-1 mM) and minimum amount of exposure duration to identify the optimal conditions for each bacterium, as shown in FIG. 11 and Table 2. To image growth patterns in different species, exponentially growing cells (OD₆₀₀ₙₘ˜0.3) were labeled, fixed, washed, “clicked” if appropriate, and imaged.

For most strains, excess dye was removed by washing the cells three to four times with 1 ml 1×PBS (NaCl 8 g/L, KCl 0.2 g/L, Na₂HPO₄-2H₂O 1.78 g/L, KH₂PO₄ 0.27 g/L, pH 7.4) and pelleting for 2-5 minutes at 10,000-16,000×g in a microfuge. When required, cells were fixed with EtOH (70%, ice-cold, 20-minute incubation). Exceptions included S. pneumoniae (1% gluteraldehyde, 20 minutes incubation) and B. subtilis (cold PBS treatment).

For dual labeling, the same procedure was followed with the addition of a second round of a short labeling pulse using the second FDAA prior to fixation. Cells were washed before, between and after each FDAA treatment with pre-warmed medium in order ensure similar labeling conditions. For triple labeling, a third round of labeling involving CDAAs was added. For dual and triple labeling of A. tumefaciens, the incubation times with each label were 5 minutes and 7 minutes, respectively.

Phase and fluorescence microscopy was performed with a Nikon® 90i Fluorescence Microscope equipped with a Plan Apo 100×/1.40 Oil Ph3 DM Objective and a Chroma 83700 triple filter cube with corresponding excitation and emission filters (DAPI for HADA/HALA; FITC for NADA/NALA; and Alexa Fluor® 488s and Texas Red® for Sulfo-Cy3 or WGA-594). All images were captured using NIS software from Nikon® and a Photometrics Cascade 1K cooled charge-coupled device camera, and were processed and analyzed using ImageJ. When a comparison was made, cultures were treated in exactly the same manner and the same parameters were applied for collecting and post processing of the microscopy data.

Long Labeling Pulses and Time-Lapse Microscopy

Exponentially growing cells were diluted to OD₆₀₀ₙₘ 0.05 in media containing half of the optimal FDAA concentration used for short labeling pulses and were grown until late exponential phase. The cells were fixed, washed and then imaged using the Nikon® 90i as described previously. For time-lapse microscopy the cells were washed with media and mounted onto LB+1% (w/v) agarose pads on 25-mm by 75-mm glass slides, sealed with 1:1:1 mixture of vasoline, lanolin and paraffin and imaged with intervals of 4 minutes (B. subtilis ΔdacA), 5 minutes (E. coli) or 10 minutes (A. tumefaciens) using a Nikon® Ti-E Inverted Fluorescence Microscope equipped with a Plan Apo 60×/1.40 Oil Ph3 DM Objective and a CFP/YFP filter cube and an Andor DU885 EMCCD Camera using CFP settings for detection of HADA.

Super-Resolution Microscopy

Structured illumination microscopy was performed using a DeltaVision® OMX Imaging System equipped with an Olympus® UPlanSApo 100×/1.40 Oil PSF Objective and a Photometrics Cascade II EMCCD Camera. The samples were excited with a laser at 405 nm and the emission was detected through a 419-465 emission filter.

Environmental Samples

HADA (500 μM+1% DMSO) and/or NADA (500 μM+1% DMSO) was added to either a 1.5 ml saliva sample from a 26-year old male or to a 1.5 ml concentrated (˜10 times) fresh water sample collected from Indiana University and incubated for 2 hours (HADA) at 37° C. or for 2 hours (HADA) and 2 hours (NADA) at 26° C., respectively. The samples were then fixed, washed and imaged.

Live-Dead Staining

E. coli cells were labeled with HADA in 0.1% DMSO. Cells were subsequently stained using the LIVE-DEAD BacLight Kit (Invitrogen) according to the manufacturer's standard protocol.

Sacculi Purification

Sacculi from cells were purified as described in Litzinger et al. (2010) J. Bacteriol. 192:3122-3143 with following modifications. Exponentially growing cells were diluted to OD₆₀₀ₙₘ 0.05 in 10 ml LB containing half of the optimum FDAA concentration+1% (v/v) DMSO and grown to late exponential phase. After aliquots were taken for whole cell imaging, cells were collected by centrifugation at 25,000×g for 15 minutes at RT and resuspended in 0.8 ml water. The suspension was added to boiling sodium dodecyl sulfate (SDS, 5% w/v) drop-wise and incubated with stirring for 30 minutes. SDS insoluble material was collected by ultracentrifugation at 39,000×g for 10 minutes at 30° C. and was resuspended in 1 ml water and boiled again in SDS (4% w/v) with stirring for 30 minutes. Samples were then washed four times in 1.5 ml water and resuspended in 1 ml 10 mM Tris-HCl pH 7.0+10 mM NaCl+0.32 M immidazole+α-amylase (100 μm/ml)+DNase I (50 μg/ml)+MgSO₄ (1 mM) and incubated for 2 hours at 37° C. Samples were pelleted and resuspended in 0.05 M Tris-HCl pH 7.8+1.4 mg/ml pronase (type XXV from Streptomyces griseus) and incubated 2 hours at 60° C. Samples were again pelleted and resuspended in 1 ml water and boiled in SDS (1% w/v) with stirring for 30 min. The sacculus preparations were washed a final time and resuspended in a minimal amount of water. When needed, sacculi were further stained with Wheat Germ Agglutinin, Alexa Fluor® 594 Conjugate (WGA-594, 15 μg/ml).

HPLC Analysis of PG and Muropeptide Identification

PG from FDAA labeled cells was purified by the boiling SDS extraction method and muramidase digestion treatment (Cellosyl) as previously described in Brown et al. (2012) Proc. Natl. Acad. Sci. USA 109:1697-1701. Solubilized muropeptide mixtures were then either directly injected into the HPLC system (native or non-reduced samples) or subjected to BH₄Na reduction as described in Brown et al. (2012), supra. Muropeptides were analyzed using a binary-pump Waters® HPLC System (Waters Corporation) fitted with a reverse phase RP18 Aeris® Peptide Column (250 mm×4.6 mm; 3.6 μm particle size) (Phenomenex) and a dual wavelength absorbance detector. Elution conditions were: flow rate 1 ml/min; temperature 35° C.; 3 minutes isocratic elution in 50 mM sodium phosphate, pH 4.35 followed by a 57 minute linear gradient to 75 mM, sodium phosphate, pH 4.95 in 15% (v/v) methanol (90 mM sodium phosphate, pH 5.2 in 30% (v/v) methanol for B. subtilis analyses), and 10 minute isocratic elution under the gradient final conditions. Elution was monitored setting one channel to 204 nm and the second to a wavelength appropriate for detection of corresponding FDAA. Muropeptides of interest were collected following HPLC separation; vacuum dried, and subjected to MALDI-mass spectrometry and electrospray ionization MS/MS as described in Brown et al. (2012), supra.

Results

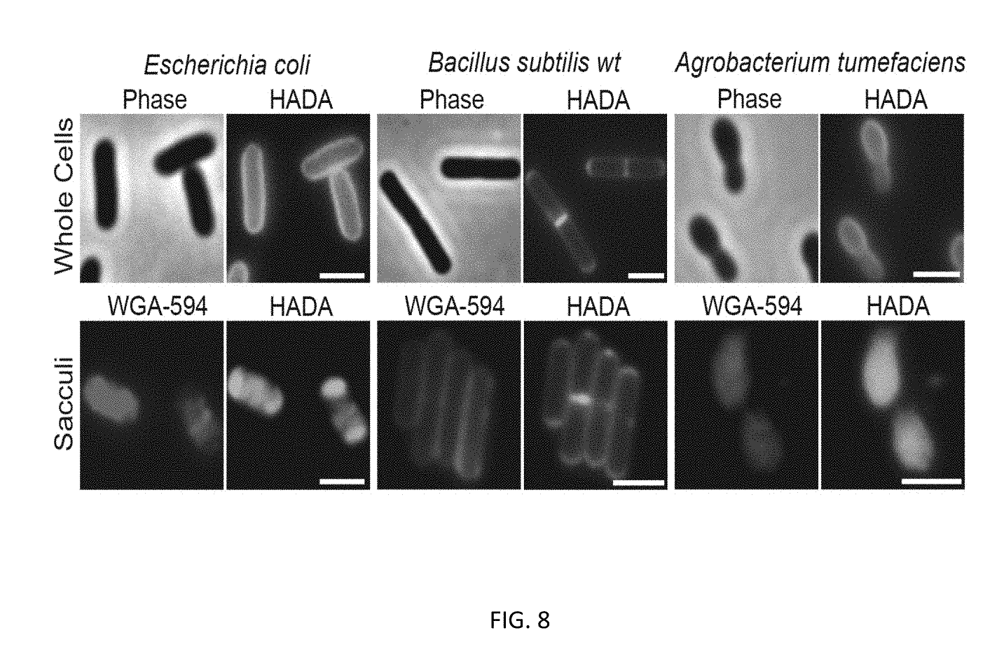

Growth of the phylogenetically diverse model species E. coli, A. tumefaciens and B. subtilis in the presence of FDAAs for as little as one generation resulted in strong peripheral and septal labeling of entire cell populations (FIG. 8) without affecting growth rate. Neither of the FLAAs prepared from 3-amino-L-alanine resulted in significant labeling, indicating that labeling is specific to the D-enantiomers. The labeling was exclusive to viable cells treated with the FDAAs and was not the result of non-specific interaction of FDAAs with the PG. Additionally, incorporation did not occur into teichoic acids for B. subtilis as indicated by identical labeling of wild-type and a ΔdltA mutant that does not D-alanylate its teichoic acids.

Retained fluorescence on the purified sacculi (FIG. 8) demonstrated that the labeling of PG by the FDAAs was covalent. HPLC analyses of muropeptides isolated from labeled cells (FIGS. 9B-C) revealed that 0.2%-2.8% of total muropeptides were modified (FIG. 9C), which is sufficient for detection in various experiments while avoiding possible toxicity issues that could result from abundant incorporation. Significantly, the FDAA-specific peaks, which were absent in samples treated with FLAAs, could be distinguished from unlabeled muropeptides at FDAA-specific absorption wavelengths (FIG. 9B). The percentage of FDAA incorporation into the total muropeptides varies among bacterial PG as revealed by HPLC analysis (FIG. 9C). MS/MS analyses of FDAA-modified muropeptides in B. subtilis indicated that FDAAs were exclusively incorporated in the fifth position of the stem peptide (FIG. 9D). Interestingly, in a ΔdacA mutant of B. subtilis, the fraction of labeled muropeptides and the fluorescent signal were substantially higher than in wild-type B. subtilis, which is likely due to the D,D-carboxypeptidase activity of DacA. In contrast, the detectable incorporation was solely at the fourth position in E. coli and A. tumefaciens (FIG. 9D). These results are in agreement with the known sites of incorporation of various natural DAAs in these species and suggest that, similar to DAAs, FDAAs incorporate mainly through periplasmic exchange reactions with the muropeptides catalyzed either by D,D-transpeptidases (e.g., in B. subtilis) or by L,D-transpeptidases (e.g., in E. coli and A. tumefaciens). This DAA-like behavior together with the ease of fluorescent detection make FDAAs a novel alternative to radioactive probes for studying in vitro and in vivo activities of PG synthesis enzymes.

Pulse-chase experiments with HADA allowed the real-time tracking of new PG incorporation during growth via time-lapse microscopy. In E. coli and B. subtilis ΔdacA (FIG. 10A), the polar caps retained the HADA signal, but the signal from the lateral walls dispersed as the cells grew, in agreement with previous reports of cell wall growth along the length of the lateral walls.

Strikingly, short labeling times (2%-8% of doubling time) using E. coli and B. subtilis ΔdacA with HADA resulted in preferential localization of the signal at the septal plane of pre-divisional cells and in punctate patterns on the lateral walls of elongating cells (FIG. 11). Super-resolution microscopy of E. coli revealed reticulated hoop-like patterns of HADA labeling around the lateral wall (FIG. 10B), supportive of bursts of PG incorporation in the side-walls. This ability of FDAAs to resolve insertion of new PG provides the first direct detection of zones of PG synthesis in a structured rather than a random pattern in E. coli, consistent with recent results following the movement of the cell wall elongation machinery. Short labeling times with A. tumefaciens, whose growth occurs predominantly from a single pole and the site of cell division while the mother cell remains inert, resulted in polar and septal labeling. Super-resolution fluorescence microscopy of labeled cells further enhanced the spatial resolution of the site of active PG synthesis (FIG. 10C).

PG labeling in three evolutionarily distant species suggested that FDAAs could specifically label the active site of PG synthesis across the entire bacterial domain. When species representing diverse phyla and modes of growth were briefly incubated with FDAAs, we observed strong labeling at the sites of cell division in actively dividing cells (FIG. 11). This septal probe incorporation was the sole mode in Synechocystis sp. PCC 6803, L. lactis and S. aureus. Super-resolution microscopy of S. aureus further highlighted the different stages of these constricting septal rings (FIG. 10D). Labeling of S. pneumoniae occurred in single or split equatorial rings depending on the length of the cell, with peripheral labeling between the split rings (FIG. 11). Labeling of S. venezuelae was predominantly apical, with some weak labeling of vegetative septa and lateral walls suggestive of a low but continuous lateral PG synthesis (FIG. 11). In C. crescentus, labeling occurred at the sites of septal elongation, lateral elongation, and stalk synthesis (FIG. 11). B. phytofirmans exhibited polar and mid-cell PG synthesis, B. conglomeratum exhibited prominent peripheral PG synthesis in addition to seemingly alternating perpendicular division planes, and V. spinosum exhibited strong peripheral PG synthesis and asymmetric septal labeling (FIG. 11).

The efficient label incorporation in all the bacteria indicates that FDAAs, therefore DAAs, incorporation is common to the bacterial domain and that FDAAs can thus be used to analyze natural bacterial populations, providing a convenient and quick standard to measure bacterial activity and to probe the diversity of growth modes in complex microbiomes. Indeed, labeling times with FDAAs as short as 2 hours revealed diverse modes of growth in saliva and freshwater samples in situ, but did not label dead cells as suggested by the strong correlation with Live-Dead staining.

Encouraged by the efficiency of FDAAs, additional and differently functionalized unnatural DAAs were prepared and used. Following a similar approach, a brighter and more versatile core fluorophore, fluorescein (emission maximum ˜515 nm, green), and its analogue, carboxytetramethylrhodamine (TAMRA, emission maximum ˜565 nm, red) were derivatized and linked with D-Lys to separate the bulky fluorophore from the DAA backbone, generating FDL and TDL (see, for example, structures in FIG. 7). Incubation of both E. coli and B. subtilis with FDL showed patterns similar to NADA, although labeling of B. subtilis was stronger than E. coli. In contrast, the larger TDL did not label E. coli cells, but labeling of B. subtilis was prominent and showed patterns similar to other FDAAs.

CDAAs, namely ethynyl-D-alanine (EDA) or azido-D-alanine (ADA) (FIG. 7), which can be specifically captured by any molecule carrying the conjugate functional group via click-chemistry also were used. Similar to FDAAs, these bioorthogonal DAAs, but not the L-enantiomer control ELA, labeled both E. coli and B. subtilis when captured by commercially available azido/alkyne fluorophores.

Furthermore, custom DAAs containing different colored fluorophores can be used sequentially to enable “virtual time-lapse microscopy.” Since addition of each new probe indicates the location and extent of PG synthetic activity during the respective labeling periods, this approach provides a chronological account of shifts in PG synthesis of individual cells over time. Examples of such serial labeling, including a combination with click chemistry, were performed in Gram-negative A. tumefaciens (FIG. 10E) and in Gram-positive S. venezuelae (FIG. 10F).

### Example 3. Synthesis of DA-DA Analogs

Amino acids, coupling reagents, and general chemicals were purchased from Sigma Aldrich, Alfa Aesar, Nova Biochem, Chem Impex, and Santa Cruz Biotech. (S)-2-Amino-4-pentynoic acid (EDA) and (R)-2-amino-4-pentynoic acid (ELA) were purchased from BoaoPharma. All solvents were HPLC grade and obtained from Omnisolv. All commercially available reagents were used as received.

¹H-NMR spectra were measured on a Varian VXR (400 MHz). ¹³C-NMR spectra were also measured on a Varian VXR (100 MHz) instrument. Proton NMR spectra were referenced to the D₂O peak at 4.79 ppm. Proton NMR spectra recorded in D₂O/CD₃CN were referenced to the D₂O peak at 4.24 ppm; ¹³C-NMR spectra recorded in D₂O/CD₃CN were referenced to the CD₃CN peak at 118.26 ppm. Mass spectral data were recorded on a Waters LCT Classic Electrospray time of flight analyzer with Agilent 1100 capillary HPLC inlet or Sciex API III electrospray quadropole with direct infusion inlet.

Analytical thin layer chromatography (TLC) was performed using Whatman glass plates coated with a 0.25 mm thickness of silica gel containing PF 254 indicator, and compounds were visualized with UV light, cerium molybdate stain, or ninhydrin stain.

Analytical high performance liquid chromatography (HPLC) was performed on an Agilent 1100 instrument with PDA and ELSD detection. Analysis was carried out using Phenomenex Luna C₁₈ reverse-phase column (5μ particle size, 100 Å pore size, 150 mm length×1.0 mm diameter) with mobile phases consisting of water and acetonitrile with 0.1% TFA. Preparatory HPLC purifications were performed with an Agilent 1100 Series HPLC purification system using a Phenomenex Luna C₁₈ reverse-phase column (5μ particle size, 100 Å pore size, 250 mm length×22 mm diameter).

Flash column chromatography was performed using Silicycle 60 Å, 35-75 μm silica gel. All compounds purified by chromatography were sufficiently pure for use in further experiments, unless otherwise noted.

### EDA-DA

EDA-DA was prepared according to Scheme (I):

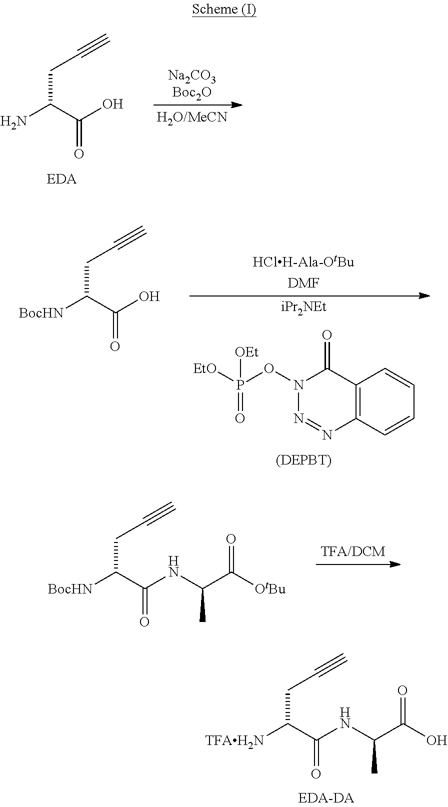

EDA/ELA (100 mg, 0.884 mmol) was added to a 25 mL round bottom flask containing sodium carbonate (234 mg, 2.21 mmol, 2.5 equiv.), and water (2.2 mL). The solution was stirred until complete dissolution was achieved and then cooled for 15 min in an ice bath. A solution of Boc anhydride (251 mg, 1.15 mmol, 1.3 equiv) in acetonitrile (2.2 mL) was added dropwise to the reaction flask. The reaction was allowed to slowly warm to room temperature overnight (14 h). The reaction was diluted with water (50 mL) and washed with diethyl ether. The aqueous layer was acidified to pH 2 with 1N HCl and extracted with ethyl acetate (3×50 mL). The organic layer was washed with brine, dried over anhydrous sodium sulfate. The solvent was removed in vacuo yielding the crude product as a clear oil that was carried on to the next step without purification.

D- or L-Ala tert-butyl ester hydrochloride (161 mg, 0.884 mmol, 1 equiv) was added to a solution of the crude reaction product dissolved in dimethylformamide (8.8 mL, 0.1 M). The flask was cooled for 10 min in an ice bath, followed by addition of diisopropylethylamine (0.46 mL, 2.65 mmol, 3 equiv.) and DEPBT (265 mg, 0.88 mmol, 1 equiv.). The solution was allowed to warm to room temperature overnight (14 h). The reaction was then diluted with ethyl acetate (50 mL) and sequentially washed with 1N HCl, water, saturated aqueous sodium bicarbonate, and brine. The organic layer was dried over anhydrous sodium sulfate and the solvent was removed in vacuo yielding the crude dipeptide product as a clear oil, which was carried on to the next step without purification.

Cleavage of the tert-butyl ester protecting group was achieved by dissolving the crude reaction mixture in a 50/50 solution of dichloromethane/trifluoroacetic acid (4 mL), followed by stirring at room temperature for 30 min. The solvent was removed in vacuo and the crude product was purified via reverse phase HPLC (3% MeCN/H₂O, 0.1% TFA, rt=4.6 min). The pure product solution was lyophilized yielding the TFA salt as a white solid (120 mg, 40% over 3 steps).

¹H NMR (400 MHz, D₂O) δ 4.43 (q, 7.3 Hz, 1H), 4.23 (t, J=7.4, 1H), 3.01-2.87 (m, 2H), 2.59 (d, J=2.7 Hz, 1H), 1.45 (d, J=7.3 Hz, 3H); ¹³C NMR (100 MHz, D₂O) δ 175.84, 167.78, 76.04, 74.33, 51.05, 48.87, 21.01, 15.90; ESI matches at 185.1 [M+H].

### ELA-LA

ELA-LA was prepared according to Scheme (II):

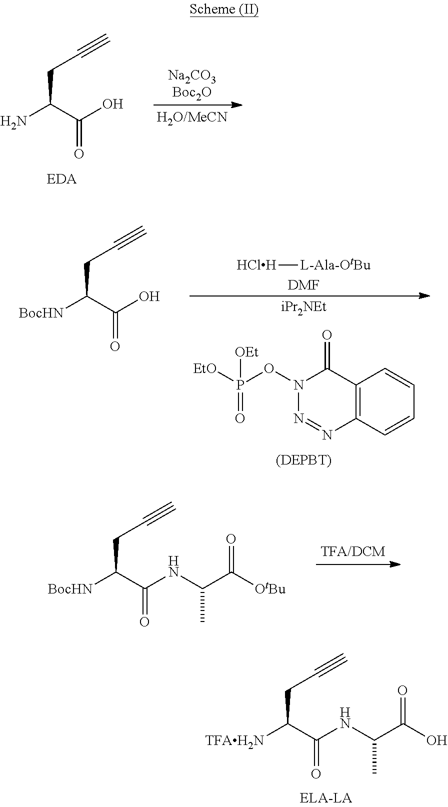

ELA-LA was prepared according to the procedure described above for EDA-DA; amino acids of the opposite absolute configuration (L-versus D-) were employed.

### DA-EDA

DA-EDA was prepared according to Scheme (III):

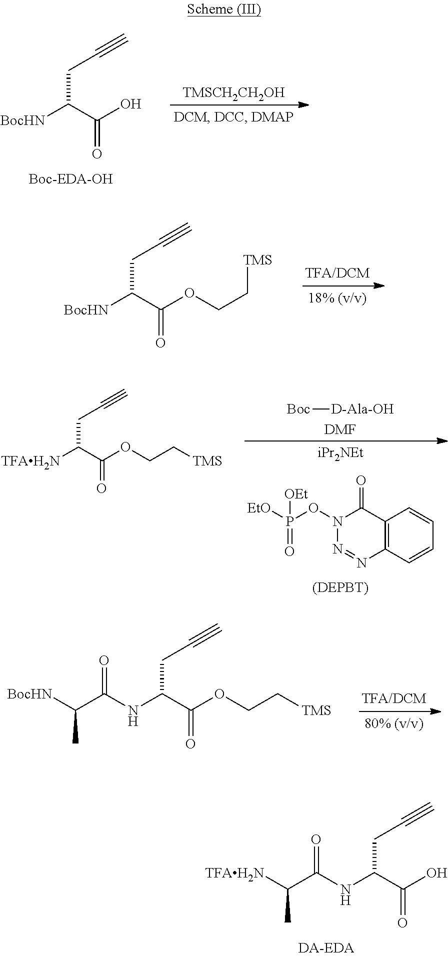

Boc-EDA-OH (3.40 g, 15.9 mmol, prepared in the same way as for EDA-DA) was added to a 250 mL round bottom flask containing 2-(trimethylsilyl)ethanol (3.77 g, 31.9 mmol, 2 equiv), and dichloromethane (80 mL, 0.2 M). The solution was cooled in an ice bath for 20 min followed by addition of dicyclohexylcarbodiimide (6.60 g, 31.9 mmol, 2 equiv.) and 4-(dimethylamino)pyridine (3.89 g, 31.9 mmol, 2 equiv.). The reaction solution was warmed to room temperature overnight (20 h). The reaction mixture was washed sequentially with 1N HCl, saturated aqueous sodium bicarbonate, and brine. The organic layer was dried over anhydrous sodium sulfate and the solvent was removed in vacuo. The crude product was purified by silica gel column chromatography (1:9 EtOAc/Hex) yielding the product as a clear oil (4.29 g, 13.695 mmol, 86%).

The purified reaction product was then dissolved in dichloromethane (DCM) and cooled in an ice bath for 15 min. Trifluoroacetic acid was then added dropwise in order to reach a final concentration of 18% TFA/DCM. The reaction was complete after 5 h as shown by TLC. The solvent was removed in vacuo and the product was added directly onto a silica gel column for purification (1:9 MeOH/EtOAc) yielding the pure TFA salt (2.66 g, 8.99 mmol, 66%).

Amino acid coupling with D-Ala-tert-butyl ester hydrochloride was performed in the same way as above for EDA-DA. The crude product (EDA-DA-OfBu) was carried on without purification.

The crude EDA-DA-OtBu dipeptide (3.46 g, 9.00 mmol) was dissolved in DCM (90 mL) and cooled in an ice bath for 15 min. TFA (72 mL) was added dropwise to provide an 80% TFA/DCM (v/v) solution and the resulting solution was warmed to room temperature overnight. The solvent was then removed in vacuo and the crude product was purified via reverse phase HPLC (10% MeCN/H₂O, 0.1% TFA, rt=3.2 min) yielding the TFA salt as a white solid (2.20 g, 6.02 mmol, 67%).

¹H NMR (400 MHz, D₂O) δ 4.60 (t, J=5.1 Hz, 1H), 4.14 (q, J=6.9 Hz, 1H), 2.88-2.72 (m, 2H), 2.44 (s, 1H), 1.56 (d, J=7.0 Hz, 3H); ¹³C NMR (100 MHz, D₂O) δ 173.27, 170.62, 79.27, 72.06, 51.65, 48.86, 20.63, 16.40; ESI matched at 185.1 [M+H].

### ADA-DA

ADA-DA was prepared according to Scheme (IV):

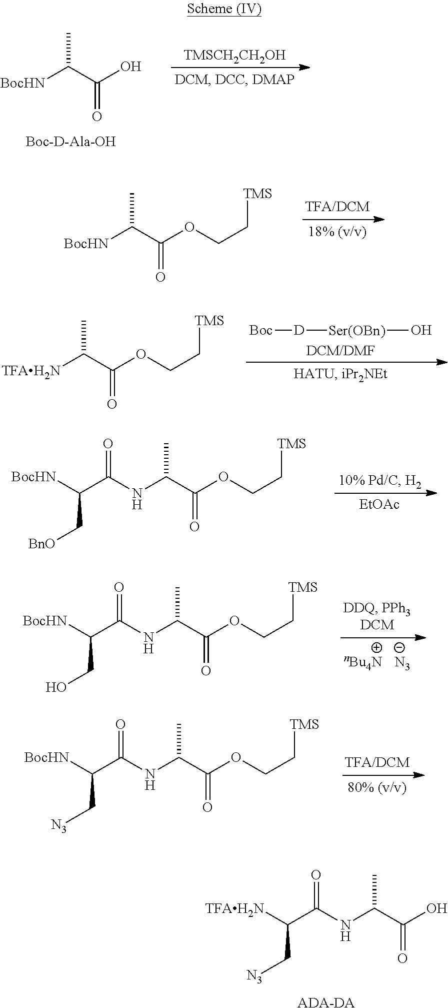

Boc-D-Ala-OH (10.0 g, 52.850 mmol) was added to a 500 mL round bottom flask containing 2-(trimethylsilyl)ethanol (12.5 g, 106 mmol, 2 equiv), and dichloromethane (265 mL, 0.2 M). The solution was cooled in an ice bath for 20 min. followed by addition of dicyclohexylcarbodiimide (21.9 g, 106 mmol, 2 equiv) and 4-(dimethylamino)pyridine (6.45 g, 52.8 mmol, 1 equiv). The resulting reaction solution was stirred and warmed to room temperature overnight (20 h). The reaction mixture was washed sequentially with 1N HCl, saturated aqueous sodium bicarbonate, and brine. The organic layer was dried over anhydrous sodium sulfate and the solvent was removed in vacuo. The crude product was purified by silica gel column chromatography (1:9 EtOAc/Hex) yielding pure Boc-D-Ala-OTMSE as a clear oil (13.2 g, 45.5 mmol, 86%).

A portion of the purified product (6.00 g, 20.7 mmol) was then dissolved in dichloromethane (207 mL) and cooled in an ice bath for 15 min. Trifluoroacetic acid (37.3 mL) was then added dropwise to make an 18% TFA/DCM (v/v) solution. The reaction was complete after 5 h as shown by TLC. The solvent was removed in vacuo and the product was added directly onto a silica gel column for purification (1:9 MeOH/EtOAc) yielding pure D-Ala-OTMSE as its TFA salt (4.36 g, 15.1 mmol, 73%).

Boc-D-Ser(OBn)-OH (1.00 g, 3.39 mmol, 1 equiv) was then added to a solution of the TFA salt (1.05 g, 3.39 mmol) in dichloromethane/dimethylformamide (1:1, 34 mL, 0.1 M) at room temperature. HATU (1.42 g, 3.73 mmol, 1.1 equiv) was then added followed by diisopropylethylamine (0.96 g, 7.4 mmol, 2.2 equiv). The resulting reaction mixture was stirred overnight (20 h). The solvent was removed by rotary evaporation and the crude product mixture was diluted with ethyl acetate (50 mL). The solution was washed sequentially with 1N HCl, saturated aqueous sodium bicarbonate, and brine. The organic layer was dried over anhydrous sodium sulfate and the solvent was removed in vacuo. The crude product was purified by silica gel column chromatography (1:4 EtOAc/Hex) to yield the pure dipeptide, Boc-D-Ser-D-Ala, (1.44 g, 3.08 mmol, 91%).

The purified dipeptide (1.30 g, 2.79 mmol) was added to a solution of 10% Pd/C (2.6 g) and ethyl acetate (50 mL) under a blanket of argon. The reaction vessel was evacuated and backfilled with hydrogen gas three times. The reaction was monitored by TLC, filtered through a pad of celite 545, and washed with MeOH. The solvent was removed in vacuo and purified via silica gel column chromatography (1:1 EtOAc/Hex) to yield the pure alcohol (0.84 g, 2.2 mmol, 80%).

To convert the free alcohol to the primary azide, a mixture of 2,3-Dichloro-5,6-dicyano-1,4-benzoquinone (106 mg, 0.47 mmol, 1.2 equiv), triphenylphosphine (122 mg, 0.47 mmol, 1.2 equiv), and tetrabutylammonium azide (133 mg, 0.469 mmol, 1.2 equiv) was dissolved in dry dichloromethane (3.9 mL) under an atmosphere of argon. A solution of the previously prepared free alcohol (147 mg, 0.39 mmol, 1 equiv) was dissolved in dry dichloromethane (1 mL) and added via syringe. The reaction was stirred for 1 h at room temperature. The reaction was then diluted with dichloromethane (50 mL), washed with brine, and dried over anhydrous sodium sulfate. The solvent was removed in vacuo and the crude product was purified via silica gel column chromatography (1:9 EtOAc/Hex) to yield the pure azide (113 mg, 0.28 mmol, 72%).

The dipeptide (113 mg, 0.28 mmol) was dissolved in DCM (2.8 mL) and cooled in an ice bath for 15 min. TFA (2.25 mL) was added to make an 80% TFA/DCM (v/v) solution and allowed to warm to room temperature overnight. The solvent was then removed in vacuo and the crude product was purified via reverse phase HPLC (10% MeCN/H₂O, 0.1% TFA, rt=3.2 min) yielding the TFA salt as a white solid (34.0 mg, 0.17 mmol, 60%).

¹H NMR (400 MHz, D₂O/CD₃CN 1:1) δ 4.42 (q, J=7.3 Hz, 1H), 4.18 (dd, J=5.8, 4.5 Hz, 1H), 3.97 (dd, J=13.7, 4.4 Hz, 1H), 3.90 (dd, J=13.7, 6.0 Hz, 1H), 1.46 (d, J=7.3 Hz, 3H); ¹³C NMR (100 MHz, D₂O/CD₃CN 1:1) δ 174.89, 166.12, 51.95, 50.32, 48.81, 16.27; ESI [M+H] matched at 202.1.

### DA-ADA

DA-ADA was prepared according to Scheme (V):

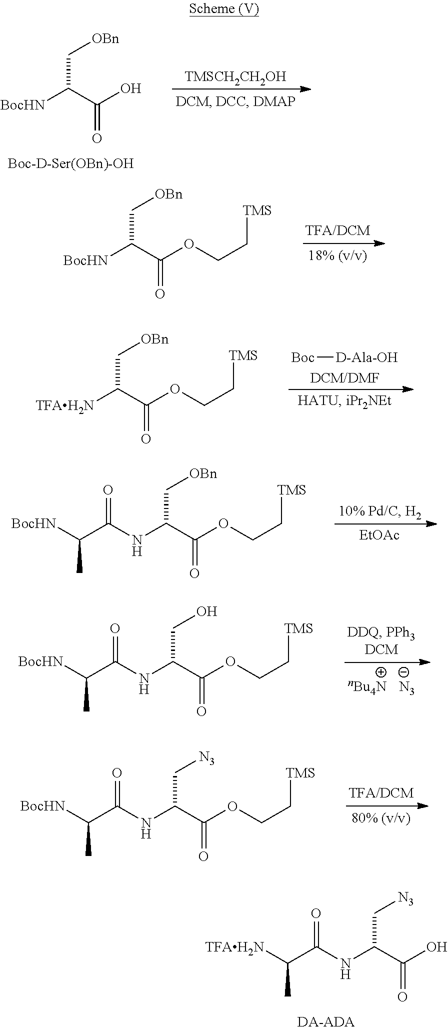

To a 25 mL round bottom flask was added Boc-D-Ser(OBn)-OH (4.00 g, 12.6 mmol), 2-(trimethylsilyl)ethanol (3.20 g, 27.1 mmol, 2 equiv), and dichloromethane (3.4 mL, 0.25 M). The solution was cooled in an ice bath for 20 min then dicyclohexylcarbodiimide (5.60 g, 27.1 mmol, 2 equiv) was added followed by 4-(dimethylamino)pyridine (1.59 g, 13.5 mmol, 1 equiv). The reaction was allowed to warm to room temperature overnight (20 h). The reaction was washed sequentially with 1N HCl, saturated aqueous sodium bicarbonate, and brine. The organic layer was dried over anhydrous sodium sulfate and the solvent was removed in vacuo. The crude product was purified by silica gel column chromatography (1:4 EtOAc/Hex) yielding the product as a clear oil.

A portion of the pure trimethylsilyl ester product was then dissolved in dichloromethane and cooled in an ice bath for 15 min. Trifluoroacetic acid was then added to make an 18% TFA/DCM solution. The reaction was complete after 5 h as shown by TLC. The solvent was removed in vacuo and the product was added directly onto a silica gel column for purification (1:9 MeOH/EtOAc) yielding the pure TFA salt (4.94 g, 12.1 mmol, 96% for two steps).

Boc-D-Ala-OH (1.32 g, 6.98 mmol, 1.1 equiv) was added to a portion of the pure TFA salt (2.60 g, 6.35 mmol) and dichloromethane/dimethylformamide (1:1, 35 mL, 0.2 M) at room temperature. HATU (2.9 g, 7.6 mmol, 1.2 equiv) was added followed by diisopropylethylamine (2.46 g, 19.0 mmol, 3 equiv) and the reaction was stirred overnight (20 h). The solvent was removed and the crude product mixture was diluted with ethyl acetate (100 mL). The solution was washed sequentially with 1N HCl, saturated aqueous sodium bicarbonate, and brine. The organic layer was dried over anhydrous sodium sulfate and the solvent was removed in vacuo. The crude product was purified by silica gel column chromatography (1:4 EtOAc/Hex) to yield the pure dipeptide (2.87 g, 6.03 mmol, 95%).

Benzyl ether deprotection and subsequent azide generation were carried using the same protocol as for ADA-DA.

The azido dipeptide (100 mg; 0.25 mmol) was dissolved in DCM (2.5 mL) and cooled in an ice bath for 15 min. TFA (2 mL) was added to make an 80% TFA/DCM (v/v) solution and allowed to warm to room temperature overnight. The solvent was then removed in vacuo and the crude product was purified via reverse phase HPLC (10% MeCN/H₂O, 0.1% TFA, rt=3.6 min) yielding the TFA salt as a white solid (29.8 mg, 0.15 mmol, 59%).

¹H NMR (400 MHz, DMSO) δ 4.50-4.43 (m, 1H), 3.87 (q, J=6.9 Hz, 1H), 3.71 (dd, J=12.8, 6.2 Hz, 1H), 3.61 (dd, J=12.8, 4.2 Hz, 1H), 1.36 (d, J=7.0 Hz, 2H); ¹³C NMR (100 MHz, DMSO) 171.06, 170.28, 52.49, 51.75, 48.61, 17.50. ESI matched at 202.1 [M+H].

### Example 4. Biological Activity of Modified Dipeptides in Labeling Bacterial PG

Cell Culture Conditions.

L2 mouse fibroblast cells were obtained from S. Weiss cultured in T-175 flasks (BD Falcon) using Dulbecco's Modified Eagle Mediuml GlutaMAX (Gibco) (DMEM) supplemented with 10% heat-inactivated Fetal Bovine Serum (FBS, HyClone) at 37° C. with 5% CO₂, and checked monthly for mycoplasma. When conducting chlamydial infections, cell medium was supplemented with 1×MEM Non-Essential Amino Acids Solution (Sigma) and 0.2 μs ml⁻¹ cycloheximide (Sigma).

Cell Infection and Bacterial Growth Conditions.

C. trachomatis serovar L2 strain 434/Bu was provided by H. Caldwell (Rocky Mountain Laboratories). Chlamydial EBs were collected from L2 cells 40 h post infection and stored at −80° C. in sucrose phosphate glutamic acid buffer (7.5% w/v sucrose, 17 mM Na₂HPO₄, 3 mM NaH₂PO₄, 5 mM L-glutamic acid, pH 7.4) until use. Stocks were titred using an infection forming unit assay (IFU). For infections, treated glass coverslips were placed in 24-well tissue culture plates (Costar) and L2 cells were plated to a confluence of ˜60-70%. Cells were washed twice with DMEM, and then infected at an MOI of between 1 and 10 with C. trachomatis. Plates were placed on a rocker for 2 h to allow for adherence of bacterial EBs to the L2 cells, and then medium was removed and replaced with infection medium (described previously) supplemented with modified D-alanine or dipeptide probes (additional supplementation with D-cycloserine (DCS, 294 μM) or ampicillin (2.8 μM) where indicated). E. coli MG1655 and B. subtilis PY79 (wild type or ΔdacA) were grown in Luria Broth (LB) (tryptone 10 g, yeast extract, 5 g NaCl 10 g) at 37° C. with aeration. S. venezuelae was grown in LB at 30° C. with aeration. For E. coli (wild type or ΔddlA, ΔddlB) and B. subtilis the minimal media used were M9+0.2% glucose ±1% LB or Spizizen's minimal medium (composition per litre, 2 g (NH₄)₂SO₄, 14 g K₂HPO₄.3H₂O, 6 g KH₂PO₄, 1 g Na-citrate.2H₂O, 0.2 g MgSO₄.7H₂O (plus tryptophan, final concentration of 50 μg ml⁻¹, and 0.5% glucose added after sterilization)), respectively. S. pneumoniae IU1945 was grown at 37° C. in brain-heart infusion (BHI) broth. When appropriate, the media were supplemented with modified D-alanine and dipeptide molecules as noted in the text.

Growth Curves with E. coli and B. subtilis.

Exponentially growing E. coli and B. subtilis were diluted to D₆₀₀ₙₘ 0.05 into wells of 24-well polystyrene plates (Falcon) containing 1 ml LB or 1 ml LB+1 mM EDA-DA or 1 mM DA-EDA. D₆₀₀ₙₘ was recorded every 45 s for 5 h in a Molecular Devices Spectramax M5 (37° C., with 5 s Automix before each measurement).

Labeling E. coli, B. Subtilis, S. Pneumonia and S. venezuelae PG.

Exponentially growing cells were diluted to D₆₀₀ₙₘ≈0.3 in media containing D-alanine or dipeptide analogues. Aliquots were taken after 5 min and 60 min. For copper-catalysed click-chemistry involving alkyne-containing PG probes and Alexa Fluor 488 Azide (Invitrogen), cells were fixed in ice-cold 70% ethanol, washed once with 1×PBS (NaCl 8 g 1⁻¹, KCl 0.2 g 1⁻¹, Na₂HP)₄-2H₂O 1.78 g 1⁻¹, KH₂PO₄ 0.27 g 1⁻¹, pH 7.4)+0.15% Polysorbate 20, once with 1×PBS+1% bovine serum albumin (BSA) and the click chemistry was performed using Click-iT Cell Reaction Buffer Kit (Invitrogen; with 10 μM azide concentration). For live-cell experiments involving azide containing PG probes, cells were washed twice with 1×PBS and copper-free click chemistry was performed using Alexa Fluor 488 DIBO Alkyne (Invitrogen; 50 μM DIBO alkyne concentration) at room temperature for 30 min. The cells were washed three times in 1×PBS and either directly imaged or resuspended in medium, grown for 30 min and then imaged.

The following live-cell experiments were done to evaluate the ability of DA-DA dipeptide probes to differentially label PG of E. coli. Referring to FIG. 15A, phase contrast and epifluorescence microscopy of E. coli cells. 5 min and 60 min aliquots were taken from cells grown with 0.5 mM alkyne containing EDA-DA, DA-EDA or as a positive control with EDA. These aliquots together with no label control were ‘clicked’ to Alexa 488 azide and imaged. When the alkyne is on the C-terminus (DA-EDA), the labeling is almost not apparent. Signal from N-terminal tag (EDA-DA) is significantly higher, but still lower than EDA and the pattern of labeling at the earlier time points is different. This is probably due to periplasmic incorporation of D-amino acids (e.g., EDA) by E. coli L,D-transpeptidases, which result in more efficient, but also non-specific labeling features. No labeling was observed when ELA-LA was used as the modified dipeptide, thereby demonstrating that labeling of E. coli PG is D-enantiomer specific (FIG. 15B). Therefore, in bacteria that have active L,D-transpeptidases, the cytoplasmic PG labeling through dipeptide probes are more reliable and specific than single D-amino acids.

The following live-cell experiments were done to evaluate the ability of DA-DA dipeptide probes to differentially label PG of diverse gram-positive bacteria. Referring to FIG. 16, phase contrast and epifluorescence microscopy experiments of labeled B. subtilis (A-C) and S. pneumoniae (D) cells are presented. In FIG. 16A, 5 min and 60 min aliquots were taken from B. subtilis wt cells grown with 0.5 mM alkyne containing EDA-DA, DA-EDA or as a positive control with EDA. These aliquots together with no label control were ‘clicked’ to Alexa 488 azide and imaged. When the alkyne is on the N-terminus (EDA-DA), features of labeling are comparable to EDA. On the other hand, the labeling with C-terminal tag (DA-EDA) is much fainter (FIG. 16A). No labeling was observed when ELA-LA was used as the modified dipeptide, thereby demonstrating that labeling of B. subtilis PG is D-enantiomer specific (FIG. 16B). Partial hollowfication of the cells on phase is an artifact of 70% EtOH fixation. In FIGS. 16C and 17, when live B. subtilis and S. pneumoniae cells labeled with azide containing ADA-DA and DA-ADA (0.4 mM & 1.6 mM for FIGS. 16C, D and 0.5 mM for FIG. 17) were non-toxically clicked to Alexa 488 DIBO alkyne, the signals from N-terminal tag ADA-DA were similarly much higher than the signal from DA-ADA labeled cells (FIG. 16C). Interestingly, the signal from DA-ADA can be elevated to the ADA-DA level, if the labeling is performed in a DacA D,D-carboxpeptidase-null mutant of B. subtilis (FIG. 16D). Since this type of click-chemistry is not toxic to cells, a pulse-chase experiment can be done, which shows the trapping of old PG at the poles of the cells (lower panel). The fluorescent images in FIG. 16 are processed the same for internal comparison. In “adjusted” images, signal intensities were lowered for comparison of labeling patterns.

Referring to FIG. 18, S. venezuelae cells were grown with the blue fluorescent D-amino acid HADA (2 h, 0.5 mM) for several generations and briefly pulsed with alkyne-containing EDA-DA (10 min, 0.5 mM) and clicked with Alexa Fluor 488 azide and imaged. The signal from EDA-DA complements the signal from HADA.

Rescue assay to test the substitution of cell's depleted DA-DA pools by exogenous DA-DA analogs.

The following experiments were done to evaluate whether growth of DA-DA ligase-null mutant bacteria could be rescued by inclusion of exogenous DA-DA analogs in growth medium. Growth rescue of a D-Ala-D-Ala ligase-null mutant of Escherichia coli was tested with varying concentrations of DA-DA, DA-EDA, DA-ADA, EDA-DA, and ADA-DA (Table 2).

For the wild-type strains, minimal DCS concentrations that inhibit growth were determined by titrating DCS in minimal medium. Growth rescue by different concentrations of DA-DA and/or its derivatives was tested using this DCS concentration (see Table 2). For initial qualitative analysis, DCS inhibition and rescue experiments were done by inoculating 1-5 μl cells from a −80° C. frozen stock. For quantitative analysis, exponentially growing cells (LB for wild-type E. coli and B. subtilis and M9+0.2% glucose+200 mM DA-DA for E. coli ΔddlA ΔddlB) were centrifuged and washed twice with M9 medium. Cells were then diluted and inoculated into wells of a 96-well plate, which contained twofold dilutions of different DA-DA and/or its derivatives for a final concentration ranging from 6.25 μM to 800 μM. For these experiments, the inoculum was D₆₀₀ₙₘ 0.025, except for the partial rescue experiment with amino-terminally modified dipeptides, rescue of which was more evident with a lower inoculum. The plates were incubated at 37° C. with aeration and the growth was monitored. Once the cultures reached saturation, D₆₀₀ₙₘ was read with a Molecular Devices Spectramax M5 and recovery was quantified by comparing average attenuance of each condition to a growth control (no drug control, or M9+0.2% glucose+200 μM DA-DA for E. coli ΔddlA ΔddlB). For clarity, the average D₆₀₀ₙₘ of the growth control was normalized to a score of 5 (that is, +++++).

While the C-terminally tagged derivatives DA-EDA and DA-ADA rescued the growth completely, the N-terminally tagged derivatives EDA-DA and ADA-DA required a small dose DA-DA (25 μM) supplementation, which is too low to rescue the growth by itself (Table 2). Similarly, E. coli and Bacillus subtilis cells are conferred auxotrophic for DA-DA by inhibiting their D-Ala-D-Ala ligase with D-cycloserine (DCS) and the inversion of the inhibition is tested by DA-DA and its analog DA-ADA. Referring to Table 2, the symbols “++,” “++++,” and “+++++” refer to positive growth, where the number of positive symbols indicate growth as a function of cell culture density (for example, “+++++”, indicates the highest culture density within the experimental set); the symbol “−” refers to no bacterial growth under the conditions evaluated; NT, not tested.

Purification and characterization of PG sacculi from E. coli and B. subtilis.

Sacculi were purified from E. coli as described (Litzinger, S. et al. “Muropeptide rescue in Bacillus subtilis involves sequential hydrolysis by beta-N-acetylglucosaminidase and N-acetylmuramyl-L-alanine amidase.” J. Bacteriol. 192:3132-3143 (2010)) with the following modifications. Exponentially growing cells were diluted to D₆₀₀ₙₘ 0.005 in 30 ml LB containing 500 μM EDA-DA and grown to late exponential phase. Cells were rapidly chilled on ice and collected by centrifugation at 25,000 g for 15 min at 4° C. and resuspended in 4 ml water. The suspension was added drop-wise to an equal volume of boiling sodium dodecyl sulphate (SDS, 8% w/v) and boiled with stirring for 45 min. SDS insoluble material was divided into 4×2 ml tubes, collected with a table-top centrifuge at 18,620 g for 20 min at room temperature and was washed three times with 2 ml water. The pelletable material was resuspended in 1 ml 10 mM Tris-HCl pH 7.0+10 mM NaCl+0.32 M imidazole+α-amylase (100 μg ml⁻¹) and incubated for 2 h at 37° C. Samples were pelleted and resuspended in 0.01 M Tris-pH 7.8+0.5% SDS+200 μg ml⁻¹ pronase (type XXV from Streptomyces griseus) and incubated 2 h at 60° C. Samples were again pelleted and resuspended in 1 ml water and boiled in an equal volume of SDS (2% w/v) with stirring for 30 min. The sacculi preparations were washed a final time and the click-chemistry was performed using Click-iT Cell Reaction Buffer Kit (Invitrogen) and Alexa Fluor 488-azide (20 μM) with 45 min incubation at room temperature. Sacculi were washed two more times and further stained with wheat germ agglutinin-Alexa Fluor 647 conjugate (WGA-647, 10 μg ml⁻¹, 15 min at room temperature) and imaged.

Unless combined with a mechanical breakage method (de Jonge, B. L., Chang, Y. S., Gage, D. & Tomasz, A. “Peptidoglycan composition of a highly methicillin-resistant Staphylococcus aureus strain. The role of penicillin binding protein 2A,” J. Biol. Chem. 267:11248-11254 (1992)), the sacculi isolation method described above applied to B. subtilis results in material trapped within the intact cell wall, presumably because of the thicker Gram-positive cell wall. This material is resistant to enzymatic digestion and causes non-specific labeling with Alexa Fluor 488-azide. Thus, the sacculi purification method for B. subtilis was modified to obtain intact sacculi that are clean of any apparent precipitates (data not shown). Exponentially growing cells were diluted to D₆₀₀ₙₘ 0.005 in 10 ml LB containing 500 μM EDA-DA and grown to late exponential phase. Cells were collected in a table-top centrifuge at room temperature, washed once with 1×PBS and combined into 3×1.7 ml tubes. The cells were resuspended in 3×1.5 ml ice-cold 70% ethanol and kept at −20° C. for 10-12 min, pelleted and washed twice with an equal volume of 1×PBS. After the last wash, click-chemistry was performed using Click-iT Cell Reaction Buffer Kit (Invitrogen) and Alexa Fluor 488-azide (20 μM) with 45 min incubation at room temperature. The cells were washed once more and resuspended in 3×1 ml 0.01 M Tris-pH 7.8+0.5% SDS+1.5 mg ml⁻¹ pronase (type XXV from Streptomyces griseus) and incubated 2 h at 60° C. The cells were pelleted and combined in 2 ml water and boiled in an equal volume of SDS (8% w/v) with stirring for 45 min. The sacculi were divided into 3×1.7 ml tubes and excess SDS was removed by washing three times with 1.5 ml water. Sacculi were further stained with wheat germ agglutinin-Alexa Fluor 647 conjugate (WGA-647, 10 μg ml⁻¹, 15 min at room temperature) and imaged.

For the lysozyme digestion experiment, a 2% agarose pad in 25 mM NaPO₄ pH 6.0, 0.5 mM MgCl₂ buffer impregnated with ˜10 mg ml⁻¹ lysozyme+200 μg ml⁻¹ mutanolysin was prepared. Quickly, EDA-DA+Alexa Fluor 488-azide labelled B. subtilis sacculi were sandwiched between a 22 mm×60 mm coverslip and a piece of the pad and imaged every 10 s for 20 min at room temperature. For the photo-bleach control, the same procedure was followed with the exclusion of the PG-digesting enzymes in the agarose pad.

Labeling chlamydial PG.

At designated time points post infection, infection medium was removed, coverslips were washed three times with PBS, and cells were fixed in methanol at room temperature for five minutes. Cells were again washed in PBS and further permeabilized in 0.5% TritonX for five minutes, and washed again. Cells were then blocked for one hour in 3% BSA before the click chemistry reaction being performed. Click-iT Cell Reaction Buffer Kit (Invitrogen) was used to carry out the click chemistry reaction with Alexa Fluor 488-azide (10 μM). The Click-iT reaction was allowed to proceed for 1 h, after which slides were washed with 3% BSA. For labelling major chlamydial outer membrane protein (MOMP) or inclusion protein A (IncA), coverslips were first blocked in DMEM supplemented with 10% heat-inactivated FBS for an hour. Coverslips were then incubated with monoclonal anti-MOMP antibody (LifeSpan Biosciences) or anti-IncA antibody (D. Rockey) diluted 1:500 in DMEM (10% FBS) for one hour, washed with DMEM (10% FBS), incubated with a secondary, chicken anti-goat or anti-rabbit IgG (respectively) conjugated to Alexa Fluor 594 (Invitrogen), diluted 1:2,000 in DMEM (10% FBS). Coverslips were washed once in DMEM (10% FBS), once in PBS, then stained with DAPI (Sigma) diluted 1:80,000 in PBS, for five minutes. Coverslips were washed one final time in PBS and mounted to glass slides with SlowFade Gold Antifade reagent (Invitrogen) for imaging.

Chlamydial Lysozyme Treatment Assay.

L2 cells were infected for 18 h, fixed, permeabilized and washed (as previously described), and blocked with 3% BSA for one hour. The click chemistry reaction was conducted (as described previously) and finally cells were suspended in 250 μl of 25 mM NaPO₄ pH 6.0, 0.5 mM MgCl₂ in the presence or absence of lysozyme (Sigma, 200 μg ml⁻¹). Cells were then rocked gently for two hours under tissue culture conditions (37° C. 5% CO₂). Counter labelling was then conducted as previously described and imaging was conducted via epifluorescence microscopy. The assay was also conducted before running the click chemistry reaction on fixed/permeabilized cells, and as the results were identical, these data were not included. Similarly, 18 h incubation in reaction buffer or lysozyme was also conducted, with results identical to the two-hour incubation.

Image Acquisition and Analysis.

Images were acquired via epifluorescence (Olympus BX50 and IX81) or confocal (Zeiss 710) laser-scanning microscopy. Image acquisition was performed with DPController (Olympus Corp.) and Zen 2009 (Carl Zeiss) software, respectively. Settings were fixed at the beginning of image acquisition. Brightness and contrast were adjusted slightly in all channels for images obtained via epifluorescence microscopy. Brightness and contrast were slightly adjusted for differential interference contrast for images taken via confocal microscopy. Image analysis was conducted with ImageJ. Deconvolution was used for generating the fluorescent images in FIG. 19. Deconvolution was conducted with AxioVision (Carl Zeiss) software using the inverse filter setting. FIG. 19A is representative of 20 inclusions viewed by confocal microscopy and over 200 inclusions viewed by epifluorescence microscopy at 18 h post infection. FIG. 20A is representative of 10 inclusions viewed by confocal microscopy and over 200 inclusions viewed by epifluorescence microscopy at 18 h post-infection. FIG. 20B is representative of 100/104 (96%) aberrant bodies induced by ampicillin treatment and viewed by confocal microscopy. All conditions were replicated technically twice and encompass at least three biological replicates of each experiment.

Phase and fluorescence microscopy of E. coli, B. subtilis and S. pneumoniae were performed with a Nikon 90i fluorescence microscope equipped with a Plan Apo×100 1.40 Oil Ph3 DM objective and a Chroma 83700 triple filter cube. Images were captured using NIS software from Nikon and a Photometrics Cascade 1K cooled charge-coupled device camera, and were processed and analysed using ImageJ.

When a comparison was made, samples were treated the same and the same parameters were applied for collecting and post processing of the microscopy data.

Chlamydial Plaque Assays.

Plaque assays were adapted from a previously described protocol (McCoy, A. J. & Maurelli, A. T. “Characterization of Chlamydia MurC-Ddl, a fusion protein exhibiting D-alanyl-D-alanine ligase activity involved in peptidoglycan synthesis and D-cycloserine sensitivity.” Mol. Microbial. 57:41-52 (2005)). Briefly, confluent monolayers of L2 mouse fibroblast cells in 24-well plates were washed twice with pre-warmed DMEM and then infected with C. trachomatis at an MOI of 1. Plates were incubated on a rocker at 37° C. with 5% CO₂ for two hours after which time, infection medium was removed and cells were overlaid with low-melting point agarose medium (0.25%) containing DMEM (1×), FBS (10%), nonessential amino acids (1×), cycloheximide (200 ng ml⁻¹), and gentamicin (20 μg ml⁻¹). Overlay medium was supplemented as indicated with DCS (30 μg ml⁻¹) and varying concentrations of amino acids and their modified derivatives. At seven days post infection, an additional agarose overlay was added to each well. At 14 days post infection, plaque formation was visualized by staining cells for three hours with 0.5% neutral red.

### REFERENCES

G. W. Liechti, E. Kuru, E. Hall′ A. Kalinda, Y. V. Brun, M. VanNieuwenhze & A. T. Maurelli, “A new metabolic cell-wall labelling method reveals peptidoglycan in Chlamydia trachomatis,” Nature, 506:507-10 (2014) [doi:10.1038/nature12892].

All of the patents, patent applications, patent application publications and other publications recited herein are hereby incorporated by reference as if set forth in their entirety.

The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the spirit and scope of the invention as set forth in the appended claims.

1. An isolated modified dipeptide consisting of a first D-amino acid covalently attached to a second D-amino acid, wherein the second D-amino acid is directly coupled to a bioorthogonal tag.

2. The isolated modified dipeptide of claim 1, wherein the second D-amino acid is ADA, AHA, EDA or EDTA.

3. The isolated modified dipeptide of claim 1, wherein the first D-amino acid of the isolated modified dipeptide is selected from the group consisting of 3-amino-D-Ala, D-Ala, D-Asp, D-Cys, D-Glu and D-Lys.

4. A muramylpentapeptide precursor unit comprising an N-acetyl muramic acid (NAM) moiety having a stem peptide of three to five amino acids, wherein one or more of the amino acids in the stem peptide comprises an isolated modified dipeptide of claim 1 and optionally an additional modified dipeptide, wherein the additional modified dipeptide includes a clickable D-amino acid.

5. A peptidoglycan unit comprising the muramylpentapeptide precursor unit of claim 4 covalently linked to an N-acetyl glucosamine (NAG) moiety.

6. A live bacterial organism comprising a bacterium having a modified cell wall comprising modified peptidoglycan containing at least one modified dipeptide according to claim 1, and optionally at least one additional modified dipeptide, wherein the at least one additional dipeptide includes a clickable D-amino acid.

7. A method of assessing bacterial cell wall synthesis in real time, the method comprising: providing live bacteria with a first amount of at least one isolated modified dipeptide of claim 1, and optionally a second amount of at least one additional modified dipeptide that includes a clickable D-amino acid, under conditions sufficient for bacterial cell wall synthesis, wherein the bacteria covalently incorporate the at least one modified dipeptide and optionally the at least one additional modified dipeptide into a stem peptide of peptidoglycan of the bacterial cell wall.

8. The method of claim 7, wherein the first amount and second amount comprise a first concentration and a second concentration, respectively, wherein the first and second concentrations range from about and including 0.10 mM to about and including 1 mM.

9. The method of claim 7, further comprising detecting the at least one isolated modified dipeptide, and optionally the at least one additional modified dipeptide incorporated into the stem peptide.

10. (canceled)

11. A method of screening for a putative cell wall-acting agent, the method comprising: co-contacting bacteria with an effective amount of an agent and an amount of at least one isolated modified dipeptide of claim 1, and optionally an amount at least one additional modified dipeptide includes a clickable D-amino acid, under conditions sufficient to permit ongoing peptidoglycan biosynthesis in a bacterial cell wall, wherein the agent comprises a cell wall-acting agent if the agent interferes with ongoing peptidoglycan biosynthesis in the bacterial cell wall.

12. The method of claim 11, further comprising: detecting one or more modified dipeptides incorporated in the bacterial cell wall.

13. The method of claim 12, wherein the detecting one or more modified dipeptides incorporated in the bacterial cell wall comprises:
(a) post-labeling the bioorthogonal tag with a label; and
(b) visualizing the one or more labeled dipeptides with microscopy.

14. The method of claim 12, further comprising: comparing the amount and/or identity of incorporated modified dipeptides in the bacterial cell wall resulting from contacting the bacteria with the agent with the corresponding amount and/or identity of incorporated modified dipeptides in a bacterial cell wall resulting from contacting the bacteria with a known cell wall-acting agent.

15. A method of screening for a putative cell wall-disrupting agent, the method comprising: contacting modified bacteria with an amount of an agent, wherein the agent comprises a cell wall-disrupting agent if the agent weakens integrity of peptidoglycan in an existing bacterial cell wall, and wherein the modified bacteria have a modified cell wall containing modified peptidoglycan having at least one stem peptide containing at least one modified dipeptide of claim 1, and optionally at least one additional modified dipeptide that includes a clickable D-amino acid.

16. The method of claim 15, further comprising: detecting one or more modified dipeptides disrupted in the bacterial cell wall.

17. The method of claim 16, wherein the detecting one or more modified dipeptides disrupted in the bacterial cell wall comprises:
(a) post-labeling the bioorthogonal tag with a label; and
(b) visualizing the one or more labeled dipeptides with microscopy.

18. The method of claim 16, further comprising: comparing the amount and/or identity of disrupted D-amino acids in the bacterial cell wall resulting from contacting the bacteria with the agent with the corresponding amount and/or identity of disrupted D-amino acids in a bacterial cell wall resulting from contacting the bacteria with a known cell wall-disrupting agent.

19. (canceled)

20. A method of identifying bacteria, the method comprising:
contacting live bacteria with an amount of at least one modified dipeptide of claim 1, and optionally an amount of at least one additional modified dipeptide that includes a clickable D-amino acid, under conditions sufficient for ongoing bacterial cell wall synthesis, wherein the bacteria covalently incorporate into peptidoglycan of a bacterial cell wall the at least one modified dipeptide, and optionally the at least one additional modified dipeptide, wherein each of the least one modified dipeptide and optionally the at least one additional modified dipeptide comprises a distinct bioorthogonal tag;
post-labeling each distinct bioorthogonal tag with spectrally distinct label; and
visualizing the spectrally distinct labels to determine an incorporation pattern of the at least one modified dipeptide, and optionally the at least one additional modified dipeptide, wherein the incorporation pattern identifies the bacteria.

21. A kit for incorporating modified dipeptides into live bacteria, the kit comprising:
at least one isolated modified dipeptide of claim 1; and
a positive bacterial control and optionally a negative bacterial control, wherein the positive bacterial control has at least one modified dipeptide of claim 1 incorporated into a stem peptide of peptidoglycan of the bacterial cell wall,
wherein the optional negative bacterial control, if included, does not have the modified dipeptide of claim 1 incorporated into a stem peptide of peptidoglycan of the bacterial cell wall.

22. The kit of claim 21, further comprising at least one clickable D-amino acid; and optionally further comprising at least one reagent for post-labeling the bioorthogonal, chemically reactive functional group.

23. (canceled)
